SEQUENCE LISTING (13 sequences)

SEQ ID NO: 1 - protein, 21 aa, Artificial Sequence
  Chemically synthesized
MEVGWYRSPFSRVVHLYRNGK

SEQ ID NO: 2 - protein, 18 aa, Artificial Sequence
  Chemically synthesized
TCFFRDHSYQEEAAVELK

SEQ ID NO: 3 - protein, 20 aa, Artificial Sequence
  Chemically synthesized
SKTSASIGSLCADARMYGVL

SEQ ID NO: 4 - DNA, 20 bp, Artificial Sequence
  Chemically synthesized
gaagctgaagaccctcagga

SEQ ID NO: 5 - DNA, 20 bp, Artificial Sequence
  Chemically synthesized
ttttcacaggggagaaatcg

SEQ ID NO: 6 - DNA, 20 bp, Artificial Sequence
  Chemically synthesized
gaactggcagaagaggcact

SEQ ID NO: 7 - DNA, 20 bp, Artificial Sequence
  Chemically synthesized
agggtctgggccatagaact

SEQ ID NO: 8 - DNA, 20 bp, Artificial Sequence
  Chemically synthesized
ccggagaggagacttcacag

SEQ ID NO: 9 - DNA, 20 bp, Artificial Sequence
  Chemically synthesized
gagcattggaaattggggta

SEQ ID NO: 10 - DNA, 20 bp, Artificial Sequence
  Chemically synthesized
aggccacaggtattttgtcg

SEQ ID NO: 11 - DNA, 20 bp, Artificial Sequence
  Chemically synthesized
gcccatcctctgtgactcat

SEQ ID NO: 12 - DNA, 19 bp, Artificial Sequence
  Chemically synthesized
catggctcgctcggtgacc

SEQ ID NO: 13 - DNA, 21 bp, Artificial Sequence
  Chemically synthesized
aatgtgaggcgggtggaactg

(19) United States
(12) Patent Application Publication
(10) Pub. No.: US 2014/0127231 A1
(43) Pub. Date: May 8, 2014

(54) p38 MAPK Pathway Inhibitors as Female-Specific Therapeutics

(71) Applicant: UNIVERSITY OF VERMONT, Burlington, VT (US)

(72) Inventors: Cory Teuscher, South Burlington, VT (US); Mercedes Rincon, Burlington, VT (US); Dimitry Krementsov, Shelburne, VT (US); Rajkumar Noubade, San Bruno, CA (US)

(73) Assignee: UNIVERSITY OF VERMONT, Burlington, VT (US)

(21) Appl. No.: 14/069,670

(22) Filed: Nov. 1, 2013

Related U.S. Application Data
(60) Provisional application No. 61/721,862, filed on Nov. 2, 2012.

(51) Int. Cl. A61K 39/395; A61K 31/7105

(52) U.S. Cl. CPC A61K 39/3955; A61K 31/7105
USPC 424/158.1; 514/17.9; 514/44 A

(57) ABSTRACT

The present invention provides compositions and methods for preventing and treating a disorder, including, for example, an autoimmune disorder (e.g. multiple sclerosis), neuroinflammation, a neurodegenerative disease, or a behavioral disorder. In one embodiment, the present invention includes administering a p38 MAPK inhibitor to a female subject in order to treat or prevent an autoimmune disorder. In another embodiment, the invention provides administering a p38 MAPK inhibitor to a specific cell population.

### CROSS-REFERENCE TO RELATED APPLICATIONS

This application claims priority to U.S. Provisional Application Ser. No. 61/721,862, filed Nov. 2, 2012, the contents of which are incorporated by reference herein in their entirety.

### STATEMENT REGARDING FEDERALLY SPONSORED RESEARCH OR DEVELOPMENT

This invention was made with government support under R21NS076200-02, AI041747, NS036526, and NS060901, awarded by the National Institutes of Health (NIH). The government has certain rights in the invention.

### BACKGROUND OF THE INVENTION

Multiple sclerosis (MS), the most common disabling neurologic disease of young adults, is considered a classical T cell-mediated disease and is characterized by demyelination, axonal damage, and progressive neurological dysfunction (Ramagopalan S V, et al., Neurol Clin. 2011 May; 29(2):207-17; Greenstein J, Dev Neurobiol. 2007 August; 67(9):1248-65). Recent genetic studies further confirmed the role of cell-mediated immunity in MS, with an emphasis on T helper cell function (Sawcer S, et al., Nature. 2011 Aug. 11; 476(7359):214-9). Despite these insights, the etiopathogenesis of this devastating disease is poorly understood and current disease-modifying therapies (DMTs) have limited efficacy.

The p38 mitogen-activated kinase (MAPK) pathway plays a prominent role in innate and adaptive immunity (Rincon M, et al., Immunol Rev. 2009 March; 228(1):212-24). p38 MAPK was identified as the target of a series of small molecules that inhibited toll-like receptor (TLR)-induced inflammatory cytokine production by macrophages (Lee J C, et al., Nature. 1994 Dec. 22-29; 372(6508):739-46). As a key regulator of pro-inflammatory cytokine production, this molecule was expected to be a promising drug target in autoimmune inflammatory disorders where these cytokines were overproduced. Indeed, animal studies have shown efficacy of p38 MAPK inhibitors in models of rheumatoid arthritis (RA), inflammatory bowel disease (IBD), and type 1 diabetes (T1D) (Liverton N J, et al., J Med Chem. 1999 Jun. 17; 42(12):2180-90; Hollenbach E, et al., FASEB J. 2004 October; 18(13):1550-2; and Ando H, et al., Life Sci. 2004 Feb. 20; 74(14):1817-27), although these compounds have not yet had success in the clinic (Genovese M C, Arthritis Rheum. 2009 February; 60(2):317-20; Hammaker D, et al., Ann Rheum Dis. 2010 January; 69 Suppl 1:i77-82). Until recently, this pathway has not been evaluated in MS or its models, despite the fact that MS shares many etiopathogenic features with these autoimmune diseases, such as activation of self-reactive T cells and augmented production of proinflammatory cytokines by innate cells (Cho J H, et al., N Engl J Med. 2011 Oct. 27; 365(17):1612-23).

Early evidence for the involvement of p38 MAPK in autoimmune neuroinflammation came from studies showing increased phosphorylation of this kinase in inflammatory cells and glia in the central nervous system (CNS) during the course of experimental autoimmune encephalomyelitis (EAE), the principal model of MS (Shin T, et al., J Neuroimmunol. 2003 July; 140(1-2):118-25). Moreover, mRNA for MAPK14 (encoding p38α) was found to be overexpressed in CNS lesions of MS patients (Lock C, et al., Nat Med. 2002 May; 8(5):500-8). Subsequently, several recent studies have documented a functional requirement for p38 MAPK signaling in EAE progression. Treatment with pharmacological inhibitors of p38 MAPK inhibited clinical signs of EAE, which correlated with inhibition of pathogenic IL-17 producing T helper cell (Th17) responses (Lu L, et al., J Immunol. 2010 Apr. 15; 184(8):4295-306; Noubade R, et al., Blood. 2011 Sep. 22; 118(12):3290-300; and Namiki K, et al., J Biol Chem. 2012 Jul. 13; 287(29):24228-38). Genetic inhibition of p38α, the predominant p38 MAPK isoform in immune cells, also potently ameliorated EAE, suggesting that p38α is the primary target underlying pharmacologic inhibition of disease (Namiki K, et al., J Biol Chem. 2012 Jul. 13; 287(29):24228-38; Huang G, et al., Nat Immunol. 2012 February; 13(2):152-61). EAE severity was also reduced by inhibition of p38 MAPK signaling specifically in T cells, either by expression of dominant negative p38 transgene in T cells, or by the mutation of a residue required for T cell-specific activation of p38α/β (Noubade R, et al., Blood. 2011 Sep. 22; 118(12):3290-300; Jirmanova L, et al., Blood, 2011 Jun. 28). Accordingly, augmentation of p38 MAPK signaling by expression of a constitutively active MKK6 transgene in T cells enhanced EAE severity (Noubade R, et al., Blood. 2011 Sep. 22; 118(12):3290-300). In contrast, Huang et at showed that genetic ablation of p38α in dendritic cells (DCs), but not in T cells or macrophages, inhibited EAE and led to impaired Th17 responses (Huang G, et al., Nat Immunol. 2012 February; 13(2):152-61). Moreover, deletion of apoptosis signal-regulating kinase 1 (ASK1), a TLR-controlled kinase that is known to activate p38 MAPK, attenuated p38 MAPK activation, EAE severity, and production of pro-inflammatory cytokines by astrocytes and microglia, without affecting peripheral T cell responses, suggesting that ASK1-dependent activation of p38 MAPK in glial cells may promote EAE pathogenesis (Guo X, et al., EMBO Mol Med. 2010 December; 2(12):504-15).

Importantly, like many other autoimmune diseases, MS is characterized by a female bias. Epidemiological studies have demonstrated a significant increase in the incidence of relapsing-remitting MS in females over the last 50 years (Ebers G C, Lancet Neurol. 2008 March; 7(3):268-77). This rate of change is suggestive of environmental factors acting specifically in females at the population level. Despite the fact that such sexual dimorphisms in autoimmunity are well-documented, the mechanistic knowledge for the development of sex-specific DMTs is lacking No study has evaluated the DMT potential of inhibiting p38 MAPK in MS, despite the fact that many compounds targeting this pathway are already approved for clinical trials in other autoimmune diseases. Additionally, relatively few studies focus on the basis of cell-specific therapies and gender-specific differences in therapeutic responses in MS or its models.

Thus, there is a need in the art for the development of compositions and methods to effectively treat autoimmune disorders, including MS. The present invention satisfies this unmet need.

### SUMMARY OF THE INVENTION

The present invention includes a method of providing a gender-specific treatment of a disorder in a female subject afflicted with the disorder. The method comprises administering a pharmaceutical composition comprising an effective amount of a p38 mitogen-activated protein kinase (MAPK) inhibitor to the female subject. In one embodiment, the subject is a human.

In one embodiment the p38 MAPK inhibitor comprises an antibody, intrabody, siRNA, ribozyme, antisense, aptamer, peptidomimetic, small molecule, or any combination thereof.

In one embodiment, the p38 MAPK inhibitor is administered to a specific cell population in the subject. In one embodiment, the p38 MAPK inhibitor is administered to a myeloid cell. In one embodiment, the myeloid cell is a macrophage, a microglia, a dendritic cell, or a neutrophil.

In one embodiment, the p38 MAPK inhibitor reduces the expression of p38 MAPK, the activation of p38 MAPK, or the activity of p38 MAPK on its effector proteins. In one embodiment, the p38 MAPK inhibitor inhibits at least one isoform of p38α, p38β, p38γ, or p38δ.

In one embodiment, the disorder is an autoimmune disorder, neuroinflammation, a neurodegenerative disorder, or a behavioral disorder. In one embodiment, the autoimmune disorder is multiple sclerosis (MS).

In one embodiment, the p38 MAPK inhibitor results in decreased cytokine production. In one embodiment, the decreased cytokine production is regulated on a post-transcriptional level.

The present invention includes a method of providing gender-specific prevention of a disorder in a female subject at risk for developing a disorder. The method comprises administering a pharmaceutical composition comprising an effective amount of a p38 mitogen-activated protein kinase (MAPK) inhibitor to the female subject. In one embodiment, the subject is a human.

In one embodiment the p38 MAPK inhibitor comprises an antibody, intrabody, siRNA, ribozyme, antisense, aptamer, peptidomimetic, small molecule, or any combination thereof.

In one embodiment, the p38 MAPK inhibitor is administered to a specific cell population in the subject. In one embodiment, the p38 MAPK inhibitor is administered to a myeloid cell. In one embodiment, the myeloid cell is a macrophage, a microglia, a dendritic cell, or a neutrophil.

In one embodiment, the p38 MAPK inhibitor reduces the expression of p38 MAPK, the activation of p38 MAPK, or the activity of p38 MAPK on its effector proteins. In one embodiment, the p38 MAPK inhibitor inhibits at least one isoform of p38α, p38β, p38γ, or p38δ.

In one embodiment, the disorder is an autoimmune disorder, neuroinflammation, a neurodegenerative disorder, or a behavioral disorder. In one embodiment, the autoimmune disorder is multiple sclerosis (MS).

In one embodiment, the p38 MAPK inhibitor results in decreased cytokine production. In one embodiment, the decreased cytokine production is regulated on a post-transcriptional level.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIG. 1A through FIG. 1C, is a set of graphs demonstrating that SB203580 (SB), a p38 MAPK inhibitor, is a female-specific therapy for EAE. FIG. 1A depicts the results of experiments where clinical EAE course in male and female B6 mice immunized with 2×MOG₃₅₋₅₅-CFA treated daily with either carrier or SB starting on D0. FIG. 1B depicts the results of experiments where clinical EAE course in female B6 mice immunized with 2×MOG₃₅₋₅₅-CFA and randomly selected for daily treatment with either carrier or SB upon reaching a clinical score≧1. FIG. 1C depicts the results of experiments where clinical course of EAE in female B6 mice immunized as in (A), and treated with either carrier or SB starting on D0 (1st arrow), treatment terminated on D31 (arrow with cross), and re-treated from D41 thru D60 (2nd arrow). The significance of the differences between the clinical courses of disease was calculated by regression analysis and best-fit curves are shown.

FIG. 2, comprising FIG. 2A through FIG. 2D, is a set of graphs demonstrating that SB treatment in vivo inhibits IL-17 production by T_{H}17 cells. Female B6 mice were immunized for EAE as described for FIG. 1 and treated daily with carrier or SB. On D30 post-immunization, CNS (brain and spinal cord) cells were harvested and analyzed by intracellular staining and flow cytometry. Relative percentages (out of CD4+TCRβ+ population) of IL-17+, IFN-γ+, or IL-17+ IFN-γ+ cells (FIG. 2A), and percentages of CD4+TCRβ+ cells (out of CD45+ cells) (FIG. 2B). DLN cells were collected on D20 and restimulated with MOG₃₅₋₅₅ for 3 days. Production of IFN-γ+(FIG. 2C) or IL-17 (FIG. 2D) was assessed by ELISA.

FIG. 3, comprising FIG. 3A through FIG. 3C, is a set of graphs demonstrating that SB treatment inhibits IL-17 production at a post-transcriptional level. Naïve CD4+ T cells were purified by negative selection and cultured under TH17 polarizing conditions for 3 days in the presence of SB20350 (5 μM if not otherwise indicated). IL-17 secretion was analyzed by ELISA (FIG. 3A). Relative IL-17 mRNA was assessed by qRT-PCR (FIG. 3B). IL-17 levels were assessed using intracellular staining and flow cytometry (FIG. 3C).

FIG. 4, comprising FIG. 4A and FIG. 4B, is a set of graphs demonstrating that manipulation of p38 activity in T cells alters EAE severity. Transgenic mice expressing dnp38-Tg (FIG. 4A) or MKK6-Tg (FIG. 4B) and WT controls were immunized with 1×PLP₁₈₀₋₂₀₉+CFA+PTX and assessed for clinical signs.

FIG. 5, comprising FIG. 5A and FIG. 5B, demonstrates that SB treatment prevents passive EAE induced by T_{H}1 cells. FIG. 5A is a schematic that illustrates that protocol of inducing passive EAE. FIG. 5B is a set of graphs depicting that SB treatment reduces EAE severity and reduces the inflammatory response.

FIG. 6, comprising FIG. 6A and FIG. 6B, is a set of graphs demonstrating the gender-specific effects of manipulating p38 MAPK activity on EAE severity. FIG. 6A depicts the disease severity in females and males induced for EAE, in SB treated and carrier treated animals. FIG. 6B is a set of graphs demonstrating that genetic augmentation of p38 activity in T cells in B10.BR enhances disease in both males and females. Wild-type (WT) and MKK6 Tg B10.BR mice were immunized using 1×CFA/MOG₇₉₋₉₆+PTX protocol and scored daily.

FIG. 7, comprising FIG. 7A and FIG. 7B, is a set of graphs demonstrating the gender-specific effects of conditional deletion of p38alpha in T cells and myeloid cells in C57BL/6 mice. FIG. 7A depicts the results of experiments where littermate p38^{f/f} (WT) and p38^{f/f} Lck-Cre Tg (p38CKO^{lck}) mice were immunized using 2×CFA-MOG₃₅₋₅₅ protocol and scored daily. FIG. 7B depicts the results of experiments where littermate p38^{f/f} (WT) and p38^{f/f} LysM-Cre Tg (p38CKO^{LysM}) mice were immunized using 2×CFA-MOG₃₅₋₅₅ protocol and scored daily.

FIG. 8, comprising FIG. 8A through FIG. 8D, is a set of graphs demonstrating the peripheral recall response in p38CKOlck mice. Female littermate p38^{f/f} (WT) and p38^{f/f} Lck-Cre Tg (p38CKO^{lck}) mice were immunized using 2×CFA/MOG₃₅₋₅₅ protocol. On D10, LN and spleen cells were isolated, restimulated with the indicated concentrations of MOG₃₅₋₅₅, and production of the following cytokines was determined by ELISA: IFNγ (FIG. 8A), IL-17 (FIG. 8B), and GM-CSF (FIG. 8C). Alternatively, LN and spleen cells were stimulated with PMA/Ionomycin in the presence of brefeldin A for 4 hours, stained and processed by intracellular cytokine staining and flow cytometry (FIG. 8D). Percentage of TCRβ+CD4+ cells positive for IFNg or IL-17 is shown.

FIG. 9, comprising FIG. 9A through FIG. 9H, is a set of graphs depicting the peripheral recall response in p38CKO^{LysM} mice. Female littermate p38^{f/f} (WT) and p38^{f/f} LysM-Cre Tg (p38CKO^{LysM}) mice were immunized using 2×CFA/MOG₃₅₋₅₅ protocol. On D10, LN and spleen cells were isolated, restimulated with the indicated concentrations of MOG₃₅₋₅₅, and production of the following cytokines was determined by ELISA: IFNg (FIG. 9A and FIG. 9D), IL-17 (FIG. 9B and FIG. 9E), and GM-CSF (FIG. 9C and FIG. 9F). Alternatively, LN and spleen cells were stimulated with PMA/Ionomycin in the presence of brefeldin A for 4 hours, stained and processed by intracellular cytokine staining and flow cytometry (FIG. 9G and FIG. 9H). Percentage of TCRβ+CD4+ cells positive for IFNg (FIG. 9G) or IL-17 (FIG. 9H) is shown.

FIG. 10, comprising FIG. 10A through FIG. 10D, is a set of graphs demonstrating the dampened CNS inflammatory response in female p38CKO^{LysM} mice. Male and female littermate p38^{f/f} (WT) and p38^{f/f} LysM-Cre Tg (p38CKO^{LysM}) mice were immunized using 2×CFA/MOG₃₅₋₅₅ protocol. On D21, mononuclear cells were isolated from the CNS using a Percoll gradient, counted (FIG. 10A), stimulated with MOG₃₅₋₅₅ for 4 hours in the presence of brefeldin A, and then analyzed by ICCS and flow cytometry. Percentage of TCRβ+CD4+ cells positive for IFNγ (FIG. 10B), IL-17 (FIG. 10C), or GM-CSF (FIG. 10D) is shown.

FIG. 11 is a set of graphs depicting the number of lymph node cells and CNS infiltrating immune cells in female mice. The data demonstrates that myeloid cell-specific deletion of p38alpha MAPK inhibits inflammatory responses in the CNS in females. Cells isolated from the CNS on Day 21, at peak inflammation.

FIG. 12 is a set of graphs that depicts the T cell inflammatory cytokine response in the CNS. The data demonstrates that myeloid cell-specific deletion of p38alpha MAPK inhibits inflammatory responses in the CNS in females. Responses were analyzed at D21, peak inflammation in CNS.

FIG. 13 is a set of graphs depicting the level of CD11b expression on CNS-infiltrating TCR-negative myeloid cells in wildtype and myeloid-specific p38alpha knockout mice. The data demonstrates that myeloid cell-specific deletion of p38alpha MAPK inhibits inflammatory responses in the CNS in females, as measured by a reduction in the activated CD11b^{hi} subset of myeloid cells. Responses were analyzed at D21, peak inflammation in CNS.

FIG. 14 is a set of graphs demonstrating that myeloid cells from male mice exhibit augmented expression of alternative isoforms of p38 MAPK.

FIG. 15, comprising FIG. 15A and FIG. 15B, is a set of graphs depicting the results of experiments, demonstrating the sex-specific modulation of EAE susceptibility by p38 MAPK. Female (FIG. 15A) and male (FIG. 15B) B6 mice were immunized using the 2×MOG₃₅₋₅₅/CFA protocol, followed by daily injections of SB203580 or carrier. Data represent 2 independent experiments, pooled. Data were analyzed as indicated in Materials and Methods. A significant difference in EAE course was observed in females [treatment, p<0.0001; time, p<0.0001; time-by-treatment interaction, p<0.0001], and in males [treatment, p=0.71; time, p<0.0001; time-by-treatment interaction, p<0.0001]. For individual time points, * ≦0.05; ** ≦0.01, *** ≦0.001, **** ≦0.0001. Numbers in parentheses indicate the total number of animals studied.

FIG. 16, comprising FIG. 16A through FIG. 16F, depicts the results of experiments demonstrating the differential control of EAE by p38α signaling in T cells, DCs, and myeloid cells. Female (left panels) and male (right panels) WT and p38CKO^{Lck} (FIG. 16A and FIG. 16B), WT and p38CKO^{Cd11c} (FIG. 16C and FIG. 16D), and WT and p38CKO^{Lysm} (FIG. 16E and FIG. 16F) mice were immunized with 2×MOG₃₅₋₅₅/CFA. Data represent two pooled independent experiments for each strain combination, and were analyzed as in FIG. 15. No significant effect of KO on EAE course was found in (FIG. 16A and FIG. 16B), females [strain, p=1.0; time, p<0.0001; time-by-treatment interaction, p=0.4], males [strain, p=0.3; time, p<0.0001; time-by-treatment interaction, p=0.6]. In (FIG. 16C and FIG. 16D), a significant effect of KO on EAE course was found in both females [strain, p=0.006; time, p<0.0001; time-by-treatment interaction, p<0.0001], and males [strain, p=0.3; time, p<0.0001; time-by-treatment interaction, p<0.0001]. In FIG. 16E and FIG. 16F, a significant effect of KO on EAE course was found in both females [strain, p=0.008; time, p<0.0001; time-by-treatment interaction, p<0.0001], and males [strain, p=0.2; time, p<0.0001; time-by-treatment interaction, p=0.02]. For individual time points, * ≦0.05; ** ≦0.01, *** ≦0.001, **** ≦0.0001. Numbers in parentheses indicate the total number of animals studied.

FIG. 17, comprising FIG. 17A through FIG. 17G, depicts the results of experiments demonstrating the ex vivo peripheral recall responses in p38CKO^{Lysm} mice. Female and male littermate WT and p38CKO^{Lysm} mice were immunized with 2×MOG₃₅₋₅₅/CFA. On D10, LN and spleen cells were isolated, restimulated with 50 μg/ml MOG₃₅₋₅₅, and production of the following cytokines was determined by ELISA: IFNγ (FIG. 17A), IL-17 (FIG. 17B), and GM-CSF (FIG. 17C). Alternatively, LN and spleen cells were stimulated with PMA/Ionomycin in the presence of brefeldin A for 4 hours, then stained and analyzed by intracellular cytokine staining and flow cytometry (FIG. 17D-FIG. 17G). Percentage of TCRβ+CD4+ cells positive for IFNγ (FIG. 17D) or IL-17 (FIG. 17E) is shown. Representative flow cytometry plots are shown for female WT (FIG. 17F) and p38CKO^{Lysm} (FIG. 17G) mice. Data are representative of two independent experiments. WT female (n=7), p38CKO^{Lysm} female (n=10), WT male (n=10), p38CKO^{Lysm} male (n=10). No significant effect of KO was detected.

FIG. 18, comprising FIG. 18A through FIG. 18L, depicts the results of experiments demonstrating the dampened CNS inflammatory response in female p38CKO^{LysM} mice. Female and male WT and p38CKO^{Lysm} mice were immunized using the 2×MOG₃₅₋₅₅/CFA protocol. On day 19, mononuclear cells were isolated from the CNS using a Percoll gradient, counted (FIG. 18A), and surface stained for the indicated markers. Cells were gated on the CD45⁺TCRβ⁻ population and expression of CD11b (CD11b^{hi} vs. CD11b^{int}) was analyzed (FIG. 18B and FIG. 18C; representative flow cytometry plots shown in FIG. 18D and FIG. 18E). Alternatively, mononuclear cells were stimulated with MOG₃₅₋₅₅ for 4 hours in the presence of brefeldin A, and then analyzed by intracellular cytokine staining and flow cytometry (FIG. 18F-FIG. 18L). Percentage of TCRβ⁺CD4⁺ cells positive for IFNγ (FIG. 18F), IL-17 (FIG. 18G), or GM-CSF (FIG. 18H) is shown. Representative flow cytometry plots are shown in (FIG. 18I-FIG. 18L). WT female (n=6), p38CKO^{Lysm} female (n=10), WT male (n=6), p38CKO^{Lysm} male (n=6).

FIG. 19, comprising FIG. 19A through FIG. 19F, depicts the results of experiments demonstrating macrophage responses to TLR stimulation in p38CKO^{Lysm} mice. Female and male WT and p38CKO^{Lysm} mice were immunized using the 2×MOG₃₅₋₅₅/CFA protocol. On day 6 thioglycolate was injected i.p., and elicited peritoneal macrophages were isolated on day 10 post-immunization. Adherent macrophages were stimulated for 30 min with 100 ng/ml LPS (FIG. 19A) or 50 μg/ml heat killed MTB (FIG. 19B), lysed, and analyzed by immunoblot for phosphorylation of p38 MAPK (p-p38). GAPDH is shown as a loading control. FIG. 19B is a composite image of two different parts of the same membrane image, as indicated by the lines, treated identically and shown at the same exposure. Alternatively, adherent macrophages were stimulated for 4 or 24 hours (as indicated) with LPS (FIG. 19C and FIG. 19D) or MTB (FIG. 19E and FIG. 19F), supernatants were collected and analyzed by ELISA for the presence of TNFα or IL-6. Data are representative of 3 independent experiments. * ≦0.05; ** ≦0.01. WT female (n=6), p38CKO^{Lysm} female (n=9), WT male (n=8), p38CKO^{Lysm} male (n=8).

FIG. 20, comprising FIG. 20A and FIG. 20B, depicts the results of experiments demonstrating the sex-specific p38α-dependent transcript modules in macrophages. Female and male WT and p38CKO^{Lysm} macrophages were isolated as described for FIG. 19, stimulated for 4 hours with 50 μg/ml heat killed MTB, RNA was extracted, reverse transcribed, and cDNA subjected to microarray analysis. (FIG. 20A) A heat map of gene expression across triplicate samples of WT and p38CKO^{LysM} (KO) macrophages from female and male mice. Each triplicate represents a pool of 2-3 different biological replicates. Expression is shown relative to the centered mean of all samples for a given gene. Genes passing the binary filter of p<0.05 and |FC|>1.5 (of KO relative to WT for either sex) are shown, ordered by FC in descending order. “Up” or “down” refers to the direction of change in KO relative to WT. (FIG. 20B) A Venn diagram indicating the overlap in p38α-dependent transcripts between females and males. Both annotated and non-annotated genes were included.

FIG. 21, comprising FIG. 21A through FIG. 21H, depicts the results of experiments demonstrating the sex-specific regulation of inflammatory gene expression in the CNS of p38CKO^{LysM} mice. Female and male WT and p38CKO^{Lysm} mice were immunized using the 2×MOG₃₅₋₅₅/CFA protocol. On day 21, mononuclear cells were isolated from the CNS using a Percoll gradient and RNA was extracted. Relative mRNA abundance of Il10 (FIG. 21A), Oas1g (FIG. 21B), Fcgr1 (FIG. 21C), Ccr1 (FIG. 21D), Ccr5 (FIG. 21E) Il1b (FIG. 21F), Tnfa (FIG. 21G), and Il6 (FIG. 21H) was quantified by qRT-PCR using the delta-delta CT method with B2m as an endogenous control. * ≦0.05. WT female (n=8), p38CKO^{Lysm} female (n=6), WT male (n=5), p38CKO^{Lysm} male (n=4).

FIG. 22, comprising FIG. 22A through FIG. 22D, depicts the results of experiments demonstrating that sex hormones contribute to the sexual dimorphism in EAE in p38CKO^{Lysm} mice. Female (left panels) and male (right panels) WT and p38CKO^{Lysm} mice underwent sham surgeries (Sham) (FIG. 22A and FIG. 22B) or gonadectomies (GndX) (FIG. 22C and FIG. 22D). Three weeks later, mice were immunized using the 2×MOG₃₅₋₅₅/CFA protocol. Data represent one independent experiment, and were analyzed as in FIG. 15. A significant effect of KO on EAE course was found in (FIG. 22A) [strain, p=0.05; time, p<0.0001; time-by-treatment interaction, p<0.0001] and in (FIG. 22D) [strain, p<0.0001; time, p<0.0001; time-by-treatment interaction, p=0.03]. No significant effect of KO on EAE course was found in FIG. 22B [strain, p=0.4; time, p<0.0001; time-by-treatment interaction, p=0.3] and in FIG. 22C [strain, p=0.7; time, p<0.0001; time-by-treatment interaction, p=0.7]. For individual time points, * ≦0.05; ** ≦0.01, *** ≦0.001, **** ≦0.0001. Numbers in parentheses indicate the total number of animals studied.

FIG. 23, comprising FIG. 23A through FIG. 23C, depicts the results of experiments demonstrating the comparable downregulation of p38α in macrophages from female or male p38CKO^{Lysm} mice. Thioglycolate-elicited peritoneal macrophages were isolated from female or male WT or p38CKO^{Lysm} mice. Cells were lysed and analyzed by immunoblotting (FIG. 23A and FIG. 23B), or RNA was isolated and abundance of Mapk14 (p38α) mRNA was analyzed using qRT-PCR and primers specific for exon 2 of Mapk14 (FIG. 23C). GAPDH is shown as a loading control, and was used to quantify relative expression of p38α in FIG. 23B. B2m was used as an endogenous control in FIG. 23C. * ≦0.05

FIG. 24, comprising FIG. 24A through FIG. 24G, depicts the results of experiments demonstrating the normal homeostasis of myeloid cells in p38CKO^{Lysm} mice. (FIG. 24A-FIG. 24G) Female and male littermate WT and p38CKO^{Lysm} mice were immunized using the 2×MOG₃₅₋₅₅/CFA protocol. On D10, LN and spleen cells were isolated and stained for the indicated surface markers. (FIG. 24A) % CD11b⁺ cells of total live cells, (FIG. 24B) % CD11c⁺ cells of total live cells, (FIG. 24C) % Ly6C⁺G⁻ (monocyte) cells of total CD11b⁺ cells, (FIG. 24D) % Ly6C⁺G⁺ (granulocyte) cells of total CD11b⁺ cells, and (FIG. 24E) % MHC Class II⁺ cells of total CD11b⁺ cells. Median fluorescence intensity (MFI) of CD80 (FIG. 24F), and CD86 (FIG. 24G) staining on CD11b⁺ cells. No significant effect of KO was seen.

FIG. 25, comprising FIG. 25A and FIG. 25B, depicts the results of experiments demonstrating that p38α signaling in myeloid cells contributes to the effector phase of EAE in females. WT female and male mice were immunized with 2×MOG₃₅₋₅₅/CFA. On day 10, effector cells from LN and spleen were harvested and restimulated ex vivo with 10 μg/ml MOG₃₅₋₅₅ and 0.5 ng/ml IL-12 for 72 hours. 20×10⁶ sex-matched effector cells were transferred to WT and p38CKO^{Lysm} female (FIG. 25A) and male (FIG. 25B) recipients. Data represent one independent experiment, and were analyzed as in FIG. 15. A significant effect of KO on EAE course was found in FIG. 25A [strain, p=0.4; time, p<0.0001; time-by-treatment interaction, p<0.01]. No significant effect of KO on EAE course was found in FIG. 25B [strain, p=0.08; time, p<0.0001; time-by-treatment interaction, p=0.2]. For individual time points, ** ≦0.01, * * * ≦0.001. Numbers in parentheses indicate the total number of animals studied.

FIG. 26, comprising FIG. 26A and FIG. 26B, depicts the results of experiments demonstrating that PTX overrides EAE resistance in female p38CKOLysm mice. Female (FIG. 26A) and male (FIG. 26B) WT and p38CKO^{Lysm} mice were immunized using the 1×MOG₃₅₋₅₅/CFA/PTX protocol. Data were analyzed as described for FIG. 15. No significant effect of KO on EAE course was found in females [strain, p=0.3; time, p<0.0001; time-by-treatment interaction, p=0.5], or males [strain, p=0.3; time, p<0.0001; time-by-treatment interaction, p=0.7]. Numbers in parentheses indicate the total number of animals studied.

### DETAILED DESCRIPTION

The present invention is based on the discovery that inhibition of p38 mitogen-activated protein kinase (p38 MAPK) reduces disease severity and inflammatory responses in autoimmune disorders. Accordingly, in one embodiment of the invention, p38 MAPK is a therapeutic target for the treatment of disorders, including but not limited to autoimmune disorders, neuroinflammation, neurodegenerative disorders, and behavioral disorders.

The present invention includes a method for preventing an autoimmune disorder in a subject where the method comprises administering a p38 MAPK inhibitor to the subject. The present invention also further includes a method of treating an autoimmune disorder in a subject comprising administering an effective amount of a p38 MAPK inhibitor to a subject afflicted with an autoimmune disorder. The present invention also includes a method of treating and/or preventing neuroinflammation in a subject. The present invention also includes a method of treating and/or preventing a neurodegenerative disorder in a subject. The present invention also includes a method of treating and/or preventing a behavioral disorder in a subject. In one embodiment, the method of the invention includes a method of preventing and/or treating multiple sclerosis (MS).

In one embodiment, the invention provides a gender-specific method comprising administering an effective amount of a p38 MAPK inhibitor to a subject, where the subject is female. As described elsewhere herein, the present invention is based on the unexpected finding that in certain instances p38 MAPK inhibition reduces disease severity and inflammatory responses specifically in female subjects. In one embodiment, the present invention includes specific inhibition of p38 MAPK in a specific cell population. For example, in certain embodiments, the method comprises administering an effective amount of a p38 MAPK inhibitor specifically to a myeloid cell, a macrophage, a microglia, a dendritic cell, or a neutrophil of a subject.

### DEFINITIONS

As used herein, each of the following terms has the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, analytical chemistry, organic chemistry, and nucleic acid chemistry and hybridization are those well-known and commonly employed in the art. Standard techniques or modifications thereof are used for chemical syntheses and chemical analyses.

The articles “a” and “an” are used herein to refer to one or to more than one (i.e. to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

Standard techniques are used for nucleic acid and peptide synthesis. The techniques and procedures are generally performed according to conventional methods in the art and various general references (e.g., Green and Sambrook, 2012, Molecular Cloning: A Laboratory Manual (Fourth Edition), Cold Spring Harbor Press, Cold Spring Harbor, N.Y.), which are provided throughout this document.

As used herein, the term “MAPK” refers to mitogen-activated protein kinases.

“Naturally-occurring” as applied to an object refers to the fact that the object can be found in nature. For example, a polypeptide or polynucleotide sequence that is present in an organism (including viruses) that can be isolated from a source in nature and which has not been intentionally modified by man is a naturally-occurring sequence.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

As used herein “endogenous” refers to any material from or produced inside an organism, cell, tissue or system.

As used herein, the term “exogenous” refers to any material introduced from or produced outside an organism, cell, tissue or system.

An “amino acid” as used herein is meant to include both natural and synthetic amino acids, and both D and L amino acids. “Standard amino acid” means any of the twenty L-amino acids commonly found in naturally occurring peptides. “Nonstandard amino acid residues” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides, and particularly at the carboxy- or amino-terminus, can be modified by methylation, amidation, acetylation or substitution with other chemical groups which can change a peptide's circulating half-life without adversely affecting activity of the peptide. Additionally, a disulfide linkage may be present or absent in the peptides.

As used herein, the terms “protein”, “peptide” and “polypeptide” are used interchangeably, and refer to a compound comprised of amino acid residues covalently linked by peptide bonds. The term “peptide bond” means a covalent amide linkage formed by loss of a molecule of water between the carboxyl group of one amino acid and the amino group of a second amino acid. A protein or peptide must contain at least two amino acids, and no limitation is placed on the maximum number of amino acids that may comprise the sequence of a protein or peptide. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Proteins” include, for example, biologically active fragments, substantially homologous proteins, oligopeptides, homodimers, heterodimers, variants of proteins, modified proteins, derivatives, analogs, and fusion proteins, among others. The proteins include natural proteins, recombinant proteins, synthetic proteins, or a combination thereof. A protein may be a receptor or a non-receptor.

As used herein, the term “fragment,” as applied to a protein or peptide, refers to a subsequence of a larger protein or peptide. A “fragment” of a protein or peptide can be at least about 20 amino acids in length; for example at least about 50 amino acids in length; at least about 100 amino acids in length, at least about 200 amino acids in length, at least about 300 amino acids in length, and at least about 400 amino acids in length (and any integer value in between).

The term “recombinant polypeptide” as used herein is defined as a polypeptide produced by using recombinant DNA methods.

As used herein, a “peptidomimetic” is a compound containing non-peptidic structural elements that is capable of mimicking the biological action of a parent peptide. A peptidomimetic may or may not comprise peptide bonds.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, intracellular antibodies (“intrabodies”), Fv, Fab and F(ab)₂, as well as single chain antibodies (scFv), camelid antibodies and humanized antibodies (Howard and Kaser, 2006 Making and Using Antibodies: A Practical Handbook, CRC Press, Boca Raton, Fla.; Harlow et al., 1999, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426). As used herein, a “neutralizing antibody” is an immunoglobulin molecule that binds to and blocks the biological activity of the antigen.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). The term “nucleic acid” typically refers to large polynucleotides.

The term “DNA” as used herein is defined as deoxyribonucleic acid.

The term “RNA” as used herein is defined as ribonucleic acid.

The term “recombinant DNA” as used herein is defined as DNA produced by joining pieces of DNA from different sources.

As used herein, the term “fragment,” as applied to a nucleic acid, refers to a subsequence of a larger nucleic acid. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides, at least about 1000 nucleotides to about 1500 nucleotides; or about 1500 nucleotides to about 2500 nucleotides; or about 2500 nucleotides (and any integer value in between).

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “oligonucleotide” typically refers to short polynucleotides, generally no greater than about 60 nucleotides. It will be understood that when a nucleotide sequence is represented by a DNA sequence (i.e., A, T, G, C), this also includes an RNA sequence (i.e., A, U, G, C) in which “U” replaces “T.”

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of an mRNA molecule also consists of the nucleotide residues of the mRNA molecule which are matched with an anti-codon region of a transfer RNA molecule during translation of the mRNA molecule or which encode a stop codon. The coding region may thus include nucleotide residues corresponding to amino acid residues which are not present in the mature protein encoded by the mRNA molecule (e.g., amino acid residues in a protein export signal sequence).

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “heterologous” as used herein is defined as DNA or RNA sequences or proteins that are derived from the different species.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous. By way of example, the DNA sequences 3′ATTGCC5′ and 3′TATGGC are 50% homologous.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

“Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

As used herein, “aptamer” refers to a small molecule that can bind specifically to another molecule. Aptamers are typically either polynucleotide- or peptide-based molecules. A polynucleotidal aptamer is a DNA or RNA molecule, usually comprising several strands of nucleic acids that adopt highly specific three-dimensional conformation designed to have appropriate binding affinities and specificities towards specific target molecules, such as peptides, proteins, drugs, vitamins, among other organic and inorganic molecules. Such polynucleotidal aptamers can be selected from a vast population of random sequences through the use of systematic evolution of ligands by exponential enrichment. A peptide aptamer is typically a loop of about 10 to about 20 amino acids attached to a protein scaffold that bind to specific ligands. Peptide aptamers may be identified and isolated from combinatorial libraries, using methods such as the yeast two-hybrid system.

“Ribozymes” as used herein are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules. Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053).

“Complementary” as used herein to refer to a nucleic acid, refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like.

The term “delivery vehicle” is used herein as a generic reference to any delivery vehicle capable of delivering a compound to a subject.

“Effective amount” or “therapeutically effective amount” are used interchangeably herein, and refer to an amount of a compound, formulation, material, or composition, as described herein effective to achieve a particular biological result. Such results may include, but are not limited to, the treatment of a disease or condition as determined by any means suitable in the art.

As used herein, the term “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

“Pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

An “individual”, “patient” or “subject”, as that term is used herein, includes a member of any animal species including, but are not limited to, birds, humans and other primates, and other mammals including commercially relevant mammals such as cattle, pigs, horses, sheep, cats, and dogs. Preferably, the subject is a human.

By the term “specifically binds,” as used herein, is meant a molecule, such as an antibody, which recognizes and binds to another molecule or feature, but does not substantially recognize or bind other molecules or features in a sample.

The phrase “inhibit,” as used herein, means to reduce a molecule, a reaction, an interaction, a gene, an mRNA, and/or a protein's expression, stability, function or activity by a measurable amount or to prevent entirely Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

The term to “treat,” as used herein, means reducing the frequency with which symptoms are experienced by a subject or administering an agent or compound to reduce the frequency and/or severity with which symptoms are experienced. As used herein, “alleviate” is used interchangeably with the term “treat.” The term “therapeutic” as used herein means a treatment and/or prophylaxis. A therapeutic effect is obtained by suppression, remission, or eradication of an autoimmune disorder.

As used herein, “treating a disease, disorder or condition” means reducing the frequency or severity with which a symptom of the disease, disorder or condition is experienced by a subject. Treating a disease, disorder or condition may or may not include complete eradication or elimination of the symptom.

As used herein, the term “salt” embraces addition salts of free acids or free bases that are compounds useful within the invention. Suitable acid addition salts may be prepared from an inorganic acid or from an organic acid. Examples of inorganic acids include hydrochloric, hydrobromic, hydriodic, nitric, carbonic, sulfuric, phosphoric acids, perchloric and tetrafluoroboronic acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Suitable base addition salts of compounds useful within the invention include, for example, metallic salts including alkali metal, alkaline earth metal and transition metal salts such as, for example, lithium, calcium, magnesium, potassium, sodium and zinc salts. Acceptable base addition salts also include organic salts made from basic amines such as, for example, N,N′-dibenzylethylenediamine, chloroprocaine, choline, diethanolamine, ethylenediamine, meglumine (N-methyl-glutamine) and procaine. All of these salts may be prepared by conventional means from the corresponding free base compound by reacting, for example, the appropriate acid or base with the corresponding free base.

As used herein, the term “liposome” refers to a microscopic, fluid-filled structure, with walls comprising one or more layers of phospholipids and molecules similar in physical and/or chemical properties to those that make up mammalian cell membranes, such as, but not limited to, cholesterol, stearylamine, or phosphatidylcholine. Liposomes can be formulated to incorporate a wide range of materials as a payload either in the aqueous or in the lipid compartments.

The term “phospholipids” refers to any member of a large class of fatlike organic compounds that in their molecular structure resemble the triglycerides, except for the replacement of a fatty acid with a phosphate-containing polar group. One end of the molecule is soluble in water (hydrophilic) and water solutions. The other, fatty acid, end is soluble in fats (hydrophobic). in watery environments, phospholipids naturally combine to form a two-layer structure (lipid bilayer) with the fat-soluble ends sandwiched in the middle and the water-soluble ends sticking out. Such lipid bilayers are the structural basis of cell membranes and liposomes.

“Instructional material,” as that term is used herein, includes a publication, a recording, a diagram, or any other medium of expression which can be used to communicate the usefulness of the composition and/or compound of the invention in a kit. The instructional material of the kit may, for example, be affixed to a container that contains the compound and/or composition of the invention or be shipped together with a container which contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention, in one embodiment, provides compositions and methods for the prevention of an autoimmune disorder in a subject at risk of developing an autoimmune disorder. In another embodiment, the invention provides compositions and methods for the treatment of an autoimmune disorder in a subject afflicted with an autoimmune disorder. The method comprises administering a therapeutically effective amount of a pharmaceutical composition comprising at least one p38 MAPK inhibitor to a subject having an autoimmune disorder, where a p38 MAPK inhibitor attenuates, prevents, or halts p38 MAPK expression, function, or activity in the subject, thereby treating the autoimmune disorder. Non-limiting examples of autoimmune disorders, for which the present invention is effective includes, but is not limited to, acute disseminated encephalomyelitis (ADEM), Addison's disease, an allergy or sensitivity, amyotrophic lateral sclerosis, anti-phospholipid antibody syndrome (APS), arthritis, autoimmune hemolytic anemia, autoimmune hepatitis, autoimmune inner ear disease, autoimmune pancreatitis, bullous pemphigoid, celiac disease, Chagas disease, chronic obstructive pulmonary disease (COPD), diabetes mellitus type 1 (IDDM), endometriosis, fibromyalgia, Goodpasture's syndrome, Graves' disease, Guillain-Barre syndrome (GBS), Hashimoto's thyroiditis, hidradenitis suppurativa, idiopathic thrombocytopenic purpura, inflammatory bowel disease, interstitial cystitis, lupus (including discoid lupus erythematosus, drug-induced lupus erythematosus. lupus nephritis, neonatal lupus, subacute cutaneous lupus erythematosus and systemic lupus erythematosus), morphea, multiple sclerosis (MS), myasthenia gravis, myopathies, narcolepsy, neuromyotonia, pemphigus vulgaris, pernicious anaemia, primary biliary cirrhosis, recurrent disseminated encephalomyelitis (multiphasic disseminated encephalomyelitis), rheumatic fever, schizophrenia, scleroderma, Sjogren's syndrome, tenosynovitis, vasculitis, and vitiligo.

In one embodiment, the autoimmune disorder is an autoimmune disorder of the central nervous system (CNS). In one embodiment, the autoimmune disorder is multiple sclerosis (MS).

In still another embodiment, the invention provides a method of treating a disease associated with elevated levels of p38 MAPK expression, function, or activity. For example, in one embodiment, the invention provides a method of treating and/or preventing neuroinflammation in a subject. In another embodiment, the invention provides a method of treating and/or preventing a neurodegenerative disorder in a subject. Non-limiting examples of neurodegenerative disorders include, but is not limited to, Alzheimer's disease, Parkinson's disease, amyotrophic lateral sclerosis (ALS), and Pick's Disease (PiD). In yet another embodiment, the invention provides a method of treating and/or preventing a behavioral disorder in a subject. Non-limiting examples of behavioral disorders include obsessive/compulsive disorder, anxiety, mood disorders, depression, bipolar disorder, attention deficit/hyperactivity disorder (ADHD), attention deficit disorder (ADD), autism, and Asperger's Syndrome.

In one embodiment, the present invention provides compositions and methods for the gender-specific prevention and/or treatment of an autoimmune disorder, neuroinflammation, a neurodegenerative disorder, or a behavioral disorder in female subjects. While p38 MAPK inhibition has been investigated as a treatment option for various disorders, exiting inhibitors have failed to show efficacy. As presented herein, a gender-specific treatment and/or prevention method is demonstrated to be effective specifically in the female population. In one embodiment, the present invention addresses the sexual dimorphism observed in the prevalence of various autoimmune disorders, including MS and rheumatoid arthritis.

The present invention is at least partly based upon the unexpected gender-specific effects of p38 MAPK inhibition on reducing disease severity and inflammatory responses in a model of MS. Accordingly, in one embodiment, the present invention comprises administering an effective amount of a p38 MAPK inhibitor to a female subject afflicted with, or at risk of developing, an autoimmune disorder. In one embodiment, the subject is a mammal, preferably a mouse, a rat, a non-human primate, or more preferably, a human.

p38 MAPK is a class of mitogen-activated protein kinases (MAPKs) that has four isoforms, p38α, p38β, p38γ, and p38δ. In one embodiment, the present invention comprises inhibition of at least one of the p38 isoforms. In one embodiment, the invention comprises inhibition of one or more of the p38 isoforms. In one embodiment, the present invention comprises inhibition of p38α and/or p38β.

Inhibiting p38 MAPK expression or activity may be accomplished using any method known to the skilled artisan. Examples of methods to inhibit p38 MAPK expression or activity include, but are not limited to decreasing expression of an endogenous p38 MAPK gene, decreasing expression of p38 MAPK mRNA, and inhibiting activity of p38 MAPK protein. Decreasing expression of an endogenous p38 MAPK gene includes providing a specific inhibitor of p38 MAPK gene expression. Decreasing expression of p38 MAPK mRNA or p38 MAPK protein includes decreasing the half-life or stability of p38 MAPK mRNA or decreasing expression of p38 MAPK mRNA. A p38 MAPK inhibitor may therefore be a compound or composition that decreases expression of a p38 MAPK gene, a compound or composition that decreases p38 MAPK mRNA half-life, stability and/or expression, or a compound or composition that inhibits p38 MAPK protein function. Examples of a p38 MAPK inhibitor include, but are not limited to, any type of compound, including a polypeptide, a peptide, a peptidomimetic, a nucleic acid, an siRNA, a microRNA, an antisense nucleic acid, an aptamer, a small molecule, an antibody, a ribozyme, an expression vector encoding a transdominant negative mutant, and combinations thereof. In one embodiment, the inhibitory effect of a therapeutic agent on p38 MAPK expression, function, or activity is indirect. In one embodiment, the present invention provides a method comprising administering a p38 MAPK inhibitor known in the art or discovered in the future. Non-limiting examples of such p38 MAPK inhibitors include SB203580, BIRB796 (Doramapimod), VX702, SB202190, LY2228820, VX745, Vinorelbine (Navelbine), PH797804, pamapimod, CMPD-1, EO1428, JX401, ML3403, RWJ67657, SB239063, SCIO469 hydrochoride, SKF86002 dihydrochloride, SX011, and TAK715.

In one embodiment, the method of the present invention comprises administering a p38 MAPK inhibitor to a specific cell or tissue type of the subject. The present invention is partly based upon the finding that inhibition of p38 MAPK activity in myeloid cells is sufficient to reduce disease severity and inflammatory responses in a model of MS. Thus, in one embodiment, the method of the invention comprises administering an effective amount of a p38 MAPK inhibitor to a myeloid cell of a subject. In another embodiment, the method of the invention comprises administering an effective amount of a p38 MAPK inhibitor to a microglial cell of a subject.

In one embodiment, the present invention includes a combinatorial treatment comprising inhibition of p38 MAPK and enhancement of hormone activity, including for example, by administration of exogenous hormone or derivatives thereof or enhancing the expression or activity of a hormone receptor. In one embodiment the present invention includes a combinatorial treatment comprising inhibition of p38 MAPK and inhibition hormone activity, including for example by inhibiting a hormone receptor or inhibiting the production or activity of a hormone. The present invention is partly based upon the finding that the sexual dimorphism of p38 MAPK inhibition is dependent upon the presence and activity of particular sex hormones in the subject.

### Compositions of the Invention

In certain embodiments, the composition of the invention comprises an inhibitor of p38 MAPK. An inhibitor of p38 MAPK is any compound, molecule, or agent that reduces, inhibits, or prevents the function of p38 MAPK. For example, an inhibitor of p38 MAPK is any compound, molecule, or agent that reduces p38 MAPK expression, activity, or both. In one embodiment, an inhibitor of p38 MAPK comprises a nucleic acid, an antisense nucleic acid, an siRNA, a ribozyme, an shRNA, a peptide, an antibody, a small molecule, an antagonist, an aptamer, or a peptidomimetic, or any combination thereof.

### p38 MAPK Inhibitors: Antibodies and Intrabodies

In one embodiment, the composition comprises an inhibitor of p38 MAPK comprising an antibody, or antibody fragment, specific for p38 MAPK. It will be appreciated by one skilled in the art that an antibody comprises any immunoglobulin molecule, whether derived from natural sources or from recombinant or synthetic sources, which is able to specifically bind to an epitope present on a target molecule. In the present invention, the target molecule may be p38 MAPK or fragments thereof. In one aspect of the invention, p38 MAPK is directly inhibited by an antibody that specifically binds to an epitope on p38 MAPK.

In certain embodiments of the invention, an antibody specific for p38 MAPK may be an antibody that is expressed as an intracellular protein. Such intracellular antibodies are also referred to as intrabodies and may comprise an Fab fragment, or preferably comprise a scFv fragment (see, e.g., Lecerf et al., Proc. Natl. Acad. Sci. USA 98:4764-49 (2001). The framework regions flanking the complementarity-determining region (CDR) regions can be modified to improve expression levels and solubility of an intrabody in an intracellular reducing environment (see, e.g., Worn et al., 2000, J. Biol. Chem. 275:2795-803). An intrabody may be directed to a particular cellular location or organelle, for example by constructing a vector that comprises a polynucleotide sequence encoding the variable regions of an intrabody that may be operatively fused to a polynucleotide sequence that encodes a particular target antigen within the cell (see, e.g., Graus-Porta et al., 1995, Mol. Cell Biol. 15:1182-91; Lener et al., 2000, Eur. J. Biochem. 267:1196-205). An intrabody may be introduced into a cell by a variety of techniques available to the skilled artisan including via a gene therapy vector, or a lipid mixture (e.g., Provectin™ manufactured by Imgenex Corporation, San Diego, Calif.), or according to photochemical internalization methods.

When the p38 MAPK inhibitor used in the compositions and methods of the invention is a polyclonal antibody (IgG), the antibody is generated by inoculating a suitable animal with a peptide comprising full length p38 MAPK. These polypeptides, or fragments thereof, may be obtained by any method known in the art, including chemical synthesis and biological synthesis, as described elsewhere herein. Antibodies produced in the inoculated animal which specifically bind to p38 MAPK or fragments thereof, are then isolated from fluid obtained from the animal. Antibodies may be generated in this manner in several non-human mammals such as, but not limited to goat, sheep, horse, camel, rabbit, and donkey. Methods for generating polyclonal antibodies are well known in the art and are described, for example in Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.

Monoclonal antibodies directed against a full length p38 MAPK or fragment thereof, may be prepared using any well-known monoclonal antibody preparation procedures, such as those described, for example, in Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y. and in Tuszynski et al. (1988, Blood, 72:109-115). Human monoclonal antibodies may be prepared by the method described in U.S. patent publication 2003/0224490. Monoclonal antibodies directed against an antigen are generated from mice immunized with the antigen using standard procedures as referenced herein. Nucleic acid encoding the monoclonal antibody obtained using the procedures described herein may be cloned and sequenced using technology which is available in the art, and is described, for example, in Wright et al. (1992, Critical Rev. in Immunol. 12(3,4):125-168) and the references cited therein.

When the antibody used in the methods of the invention is a biologically active antibody fragment or a synthetic antibody corresponding to antibody to a full length p38 MAPK or fragments thereof, the antibody is prepared as follows: a nucleic acid encoding the desired antibody or fragment thereof is cloned into a suitable vector. The vector is transfected into cells suitable for the generation of large quantities of the antibody or fragment thereof. DNA encoding the desired antibody is then expressed in the cell thereby producing the antibody. The nucleic acid encoding the desired peptide may be cloned and sequenced using technology which is available in the art, and described, for example, in Wright et al. (1992, Critical Rev. in Immunol. 12(3,4):125-168) and the references cited therein. Alternatively, quantities of the desired antibody or fragment thereof may also be synthesized using chemical synthesis technology. If the amino acid sequence of the antibody is known, the desired antibody can be chemically synthesized using methods known in the art as described elsewhere herein.

The present invention also includes the use of humanized antibodies specifically reactive with an epitope present on a target molecule. These antibodies are capable of binding to the target molecule. The humanized antibodies useful in the invention have a human framework and have one or more complementarity determining regions (CDRs) from an antibody, typically a mouse antibody, specifically reactive with a targeted cell surface molecule.

When the antibody used in the invention is humanized, the antibody can be generated as described in Queen, et al. (U.S. Pat. No. 6,180,370), Wright et al., (supra) and in the references cited therein, or in Gu et al. (1997, Thrombosis and Hematocyst 77(4):755-759), or using other methods of generating a humanized antibody known in the art. The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to humanized immunoglobulin coding sequences, including naturally-associated or heterologous promoter regions. The expression control sequences can be eukaryotic promoter systems in vectors capable of transforming or transfecting eukaryotic host cells, or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference).

Human constant region (CDR) DNA sequences from a variety of human cells can be isolated in accordance with well-known procedures. Preferably, the human constant region DNA sequences are isolated from immortalized B-cells as described in WO 87/02671. CDRs useful in producing the antibodies of the present invention may be similarly derived from DNA encoding monoclonal antibodies capable of binding to the target molecule. Such humanized antibodies may be generated using well known methods in any convenient mammalian source capable of producing antibodies, including, but not limited to, mice, rats, camels, llamas, rabbits, or other vertebrates. Suitable cells for constant region and framework DNA sequences and host cells in which the antibodies are expressed and secreted, can be obtained from a number of sources, such as the American Type Culture Collection, Manassas, Va.

One of skill in the art will further appreciate that the present invention encompasses the use of antibodies derived from camelid species. That is, the present invention includes, but is not limited to, the use of antibodies derived from species of the camelid family. As is well known in the art, camelid antibodies differ from those of most other mammals in that they lack a light chain, and thus comprise only heavy chains with complete and diverse antigen binding capabilities (Hamers-Casterman et al., 1993, Nature, 363:446-448). Such heavy-chain antibodies are useful in that they are smaller than conventional mammalian antibodies, they are more soluble than conventional antibodies, and further demonstrate an increased stability compared to some other antibodies. Camelid species include, but are not limited to Old World camelids, such as two-humped camels (C. bactrianus) and one humped camels (C. dromedarius). The camelid family further comprises New World camelids including, but not limited to llamas, alpacas, vicuna and guanaco. The production of polyclonal sera from camelid species is substantively similar to the production of polyclonal sera from other animals such as sheep, donkeys, goats, horses, mice, chickens, rats, and the like. The skilled artisan, when equipped with the present disclosure and the methods detailed herein, can prepare high-titers of antibodies from a camelid species.

V_{H} proteins isolated from other sources, such as animals with heavy chain disease (Seligmann et al., 1979, Immunological Rev. 48:145-167, incorporated herein by reference in its entirety), are also useful in the compositions and methods of the invention. The present invention further comprises variable heavy chain immunoglobulins produced from mice and other mammals, as detailed in Ward et al. (1989, Nature 341:544-546, incorporated herein by reference in its entirety). Briefly, V_{H} genes are isolated from mouse splenic preparations and expressed in E. coli. The present invention encompasses the use of such heavy chain immunoglobulins in the compositions and methods detailed herein.

Antibodies useful as p38 MAPK inhibitors in the invention may also be obtained from phage antibody libraries. To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (2001, Molecular Cloning: A Laboratory Manual, Cold Spring Harbor Laboratory Press, Cold Spring Harbor, N.Y.).

Bacteriophage which encode the desired antibody, may be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art and are described for example, in Wright et al., (supra).

Processes such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage which encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al., 1991, J. Mol. Biol. 222:581-597. Panning of phage so generated for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, 1995, Nature Medicine 1:837-839; de Kruif et al., 1995, J. Mol. Biol. 248:97-105).

Once expressed, whole antibodies, dimers derived therefrom, individual light and heavy chains, or other forms of antibodies can be purified according to standard procedures known in the art. Such procedures include, but are not limited to, ammonium sulfate precipitation, the use of affinity columns, routine column chromatography, gel electrophoresis, and the like (see, generally, R. Scopes, “Protein Purification”, Springer-Verlag, N.Y. (1982)). Substantially pure antibodies of at least about 90% to 95% homogeneity are preferred, and antibodies having 98% to 99% or more homogeneity most preferred for pharmaceutical uses. Once purified, the antibodies may then be used to practice the method of the invention, or to prepare a pharmaceutical composition useful in practicing the method of the invention.

The antibodies of the present invention can be assayed for immunospecific binding by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g, Current Protocols in Molecular Biology, (Ausubel et al., eds., 2002, Greene Publishing Associates and Wiley-Interscience, New York).

p38 MAPK Inhibitors: siRNA

In one embodiment, siRNA is used to decrease the level of p38 MAPK protein. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, Pa. (2003); and Gregory J. Hannon, Ed., RNAi A Guide to Gene Silencing, Cold Spring Harbor Laboratory Press, Cold Spring Harbor, N.Y. (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the present invention also includes methods of decreasing levels of p38 MAPK protein using RNAi technology.

Following the generation of the siRNA polynucleotide of the present invention, a skilled artisan will understand that the siRNA polynucleotide will have certain characteristics that can be modified to improve the siRNA as a therapeutic compound. Therefore, the siRNA polynucleotide may be further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrwal et al., 1987 Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

Any polynucleotide of the invention may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl- methyl-, thio- and other modified forms of adenine, cytidine, guanine, thymine, and uridine.

### p38 MAPK Inhibitors: Antisense Nucleic Acids

In one embodiment of the invention, an antisense nucleic acid sequence which is expressed by a plasmid vector is used to inhibit p38 MAPK expression. The antisense expressing vector is used to transfect a mammalian cell or the mammal itself, thereby causing reduced endogenous expression of p38 MAPK.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

### p38 MAPK Inhibitors: Ribozymes

Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053). Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). A major advantage of this approach is the fact that ribozymes are sequence-specific.

There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules.

In one embodiment of the invention, a ribozyme is used to inhibit p38 MAPK expression. Ribozymes useful for inhibiting the expression of a target molecule may be designed by incorporating target sequences into the basic ribozyme structure which are complementary, for example, to the mRNA sequence of p38 MAPK of the present invention. Ribozymes targeting p38 MAPK may be synthesized using commercially available reagents (Applied Biosystems, Inc., Foster City, Calif.) or they may be genetically expressed from DNA encoding them.

### p38 MAPK Inhibitors: Peptides

The present invention includes a p38 MAPK inhibitor, where the inhibitor is a peptide. For example, in one embodiment, the peptide reduces or inhibits p38 MAPK activity. In one embodiment, the peptide binds, directly or indirectly, with p38 MAPK, thereby inhibiting the activation of p38 MAPK or inhibiting the activity of p38 MAPK on effector proteins. In another embodiment, the peptide comprises a mutant p38 MAPK, for example a dominant negative p38 MAPK.

When the p38 MAPK inhibitor is a peptide, the peptide may be chemically synthesized by Merrifield-type solid phase peptide synthesis. This method may be routinely performed to yield peptides up to about 60-70 residues in length, and may, in some cases, be utilized to make peptides up to about 100 amino acids long. Larger peptides may also be generated synthetically via fragment condensation or native chemical ligation (Dawson et al., 2000, Ann. Rev. Biochem. 69:923-960). An advantage to the utilization of a synthetic peptide route is the ability to produce large amounts of peptides, even those that rarely occur naturally, with relatively high purities, i.e., purities sufficient for research, diagnostic or therapeutic purposes.

Solid phase peptide synthesis is described by Stewart et al. in Solid Phase Peptide Synthesis, 2nd Edition, 1984, Pierce Chemical Company, Rockford, Ill.; and Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and coupling thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group, such as formation into a carbodiimide, a symmetric acid anhydride, or an “active ester” group, such as hydroxybenzotriazole or pentafluorophenyl esters.

Examples of solid phase peptide synthesis methods include the BOC method, which utilizes tert-butyloxcarbonyl as the α-amino protecting group, and the FMOC method, which utilizes 9-fluorenylmethyloxcarbonyl to protect the α-amino of the amino acid residues. Both methods are well-known by those of skill in the art.

Incorporation of N- and/or C-blocking groups may also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin, so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB (divinylbenzene), resin, which upon hydrofluoric acid (HF) treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by trifluoroacetic acid (TFA) in dicholoromethane. Esterification of the suitably activated carboxyl function, e.g. with dicyclohexylcarbodiimide (DCC), can then proceed by addition of the desired alcohol, followed by de-protection and isolation of the esterified peptide product.

Incorporation of N-terminal blocking groups may be achieved while the synthesized peptide is still attached to the resin, for instance by treatment with a suitable anhydride and nitrile. To incorporate an acetyl blocking group at the N-terminus, for instance, the resin-coupled peptide can be treated with 20% acetic anhydride in acetonitrile. The N-blocked peptide product may then be cleaved from the resin, de-protected and subsequently isolated.

Prior to its use as a p38 MAPK inhibitor in accordance with the invention, a peptide is purified to remove contaminants. Any one of a number of a conventional purification procedures may be used to attain the required level of purity including, for example, reversed-phase high-pressure liquid chromatography (HPLC) using an alkylated silica column such as C₄-, C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate polypeptides based on their charge. Affinity chromatography is also useful in purification procedures.

Peptides may be modified using ordinary molecular biological techniques to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The polypeptides useful in the invention may further be conjugated to non-amino acid moieties that are useful in their application. In particular, moieties that improve the stability, biological half-life, water solubility, and immunologic characteristics of the peptide are useful. A non-limiting example of such a moiety is polyethylene glycol (PEG).

### p38 MAPK Inhibitors: Nucleic Acids and Vectors

The present invention includes a nucleic acid encoding a desired peptide. Nucleic acids encoding the desired peptide or equivalents may be replicated in wide variety of cloning vectors in a wide variety of host cells.

In brief summary, the expression of natural or synthetic nucleic acids encoding a desired peptide will typically be achieved by operably linking a nucleic acid encoding the desired peptide or portions thereof to a promoter, and incorporating the construct into an expression vector. The vectors can be suitable for replication and integration. Typical cloning vectors contain transcription and translation terminators, initiation sequences, and promoters useful for regulation of the expression of the desired nucleic acid sequence.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors. In some aspects, the expression vector is selected from the group consisting of a viral vector, a bacterial vector, and a mammalian cell vector. Numerous expression vector systems exist that comprise at least a part or all of the compositions discussed above. Prokaryote- and/or eukaryote-vector based systems can be employed to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al., Molecular Cloning: A Laboratory Manual, volumes 1-3 (3^{rd} ed., Cold Spring Harbor Press, NY 2001), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (e.g., WO 01/96584; WO 01/29058; and U.S. Pat. No. 6,326,193).

For expression of the polypeptides of the invention or portions thereof, at least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either co-operatively or independently to activate transcription.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” e.g., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR, in connection with the compositions disclosed herein (e.g., U.S. Pat. No. 4,683,202, U.S. Pat. No. 5,928,906).

Naturally, it will be important to employ a promoter and/or enhancer that effectively directs the expression of the DNA segment in the cell type, organelle, and organism chosen for expression. The promoters employed may be constitutive, tissue-specific, inducible, and/or useful under the appropriate conditions to direct high level expression of the introduced DNA segment, such as is advantageous in the large-scale production of recombinant proteins and/or polypeptides. The promoter may be heterologous or endogenous. In one embodiment, the promoter is a cell-specific or tissue-specific promoter, thereby directing expression in a particular population. In one embodiment, the vector directs expression of the p38 MAPK inhibitor specifically in myeloid cells. In another embodiment, the vector directs expression of the p38 MAPK inhibitor specifically in T cells.

An example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the muscle creatine promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess the expression of the peptides of the invention or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

### p38 MAPK Inhibitors: Small Molecules

When the p38 MAPK inhibitor is a small molecule, a small molecule inhibitor may be obtained using standard methods known to the skilled artisan. Such methods include chemical organic synthesis or biological means. Biological means include purification from a biological source, recombinant synthesis and in vitro translation systems, using methods well known in the art. In one embodiment, a small molecule inhibitor of the invention comprises an organic molecule, inorganic molecule, biomolecule, synthetic molecule, and the like.

Combinatorial libraries of molecularly diverse chemical compounds potentially useful in treating a variety of diseases and conditions are well known in the art as are method of making said libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

Small molecule inhibitors of p38 MAPK are known in the art. Exemplary small molecule p38 MAPK inhibitors include, but are not limited to SB203580, BIRB796 (Doramapimod), VX702, SB202190, LY2228820, VX745, Vinorelbine (Navelbine), PH797804, pamapimod, CMPD-1, EO1428, JX401, ML3403, RWJ67657, SB239063, SCIO469 hydrochoride, SKF86002 dihydrochloride, SX011, and TAK715.

Where tautomeric forms may be present for any of the inhibitors described herein, each and every tautomeric form is intended to be included in the present invention, even though only one or some of the tautomeric forms may be explicitly depicted.

The invention also includes any or all of the stereochemical forms, including any enantiomeric or diasteriomeric forms of the inhibitors described. The recitation of the structure or name herein is intended to embrace all possible stereoisomers of inhibitors depicted. All forms of the inhibitors are also embraced by the invention, such as crystalline or non-crystalline forms of the inhibitors. Compositions comprising an inhibitor of the invention are also intended, such as a composition of substantially pure inhibitor, including a specific stereochemical form thereof, or a composition comprising mixtures of inhibitors of the invention in any ratio, including two or more stereochemical forms, such as in a racemic or non-racemic mixture.

In one embodiment, the small molecule inhibitor of the invention comprises an analog or derivative of an inhibitor described herein.

In one embodiment, the small molecules described herein are candidates for derivatization. As such, in certain instances, the analogs of the small molecules described herein that have modulated potency, selectivity, and solubility are included herein and provide useful leads for drug discovery and drug development. Thus, in certain instances, during optimization new analogs are designed considering issues of drug delivery, metabolism, novelty, and safety.

In some instances, small molecule inhibitors described herein are derivatized/analoged as is well known in the art of combinatorial and medicinal chemistry. The analogs or derivatives can be prepared by adding and/or substituting functional groups at various locations. As such, the small molecules described herein can be converted into derivatives/analogs using well known chemical synthesis procedures. For example, all of the hydrogen atoms or substituents can be selectively modified to generate new analogs. Also, the linking atoms or groups can be modified into longer or shorter linkers with carbon backbones or hetero atoms. Also, the ring groups can be changed so as to have a different number of atoms in the ring and/or to include hetero atoms. Moreover, aromatics can be converted to cyclic rings, and vice versa. For example, the rings may be from 5-7 atoms, and may be homocycles or heterocycles.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule inhibitors described herein or can be based on a scaffold of a small molecule inhibitor described herein, but differing from it in respect to certain components or structural makeup, which may have a similar or opposite action metabolically. An analog or derivative of any of a small molecule inhibitor in accordance with the present invention can be used to reduce skin pigmentation.

In one embodiment, the small molecule inhibitors described herein can independently be derivatized/analoged by modifying hydrogen groups independently from each other into other substituents. That is, each atom on each molecule can be independently modified with respect to the other atoms on the same molecule. Any traditional modification for producing a derivative/analog can be used. For example, the atoms and substituents can be independently comprised of hydrogen, an alkyl, aliphatic, straight chain aliphatic, aliphatic having a chain hetero atom, branched aliphatic, substituted aliphatic, cyclic aliphatic, heterocyclic aliphatic having one or more hetero atoms, aromatic, heteroaromatic, polyaromatic, polyamino acids, peptides, polypeptides, combinations thereof, halogens, halo-substituted aliphatics, and the like. Additionally, any ring group on a compound can be derivatized to increase and/or decrease ring size as well as change the backbone atoms to carbon atoms or hetero atoms.

### Methods of the Invention

### Methods of Preventing and Treating Autoimmune Disorders

In one embodiment, the invention describes a method for the delivery of a p38 MAPK inhibitor for the treatment or prevention of autoimmune disorders. Non-limiting examples of autoimmune disorders, in which the present method would be effective includes acute disseminated encephalomyelitis (ADEM), Addison's disease, an allergy or sensitivity, amyotrophic lateral sclerosis, anti-phospholipid antibody syndrome (APS), arthritis, autoimmune hemolytic anemia, autoimmune hepatitis, autoimmune inner ear disease, autoimmune pancreatitis, bullous pemphigoid, celiac disease, Chagas disease, chronic obstructive pulmonary disease (COPD), diabetes mellitus type 1 (IDDM), endometriosis, fibromyalgia, Goodpasture's syndrome, Graves' disease, Guillain-Barre syndrome (GBS), Hashimoto's thyroiditis, hidradenitis suppurativa, idiopathic thrombocytopenic purpura, inflammatory bowel disease, interstitial cystitis, lupus (including discoid lupus erythematosus, drug-induced lupus erythematosus. lupus nephritis, neonatal lupus, subacute cutaneous lupus erythematosus and systemic lupus erythematosus), morphea, multiple sclerosis (MS), myasthenia gravis, myopathies, narcolepsy, neuromyotonia, pemphigus vulgaris, pernicious anaemia, primary biliary cirrhosis, recurrent disseminated encephalomyelitis (multiphasic disseminated encephalomyelitis), rheumatic fever, schizophrenia, scleroderma, Sjogren's syndrome, tenosynovitis, vasculitis, and vitiligo. In one embodiment, the autoimmune disorder is an autoimmune disorder of the central nervous system (CNS). In one embodiment, the method of the invention prevents and/or treats MS.

In still another embodiment, the invention provides a method of treating a disease associated with elevated levels of p38 MAPK expression, function, or activity. For example, in one embodiment, the invention provides a method of treating and/or preventing neuroinflammation in a subject. In another embodiment, the invention provides a method of treating and/or preventing a neurodegenerative disorder in a subject. Non-limiting examples of neurodegenerative disorders include Alzheimer's disease, Parkinson's disease, amyotrophic lateral sclerosis (ALS), and Pick's Disease (PiD). In yet another embodiment, the invention provides a method of treating and/or preventing a behavioral disorder in a subject. Non-limiting examples of behavioral disorders include obsessive/compulsive disorder, anxiety, mood disorders, depression, bipolar disorder, attention deficit/hyperactivity disorder (ADHD), attention deficit disorder (ADD), autism, and Asperger's Syndrome.

As described elsewhere herein, in one embodiment, the method of the present invention is gender-specific. Accordingly, in one embodiment, the present invention includes a method comprising administering an effective amount of a p38 MAPK inhibitor to a female subject.

Inhibiting p38 MAPK expression, function, or activity can be accomplished using any method known to the skilled artisan, as described elsewhere herein. Decreasing expression of an endogenous p38 MAPK gene includes providing a specific inhibitor of p38 MAPK gene expression. p38 MAPK inhibition may be accomplished either directly or indirectly. For example, p38 MAPK may be directly inhibited by compounds or compositions that directly interact with p38 MAPK protein, such as antibodies. Alternatively, p38 MAPK may be inhibited indirectly by compounds or compositions that inhibit p38 MAPK downstream effectors, or upstream regulators which up-regulate p38 MAPK expression.

In one embodiment, the method of the invention comprises administering a p38 MAPK inhibitor known in the art or discovered in the future. Non-limiting examples of such p38 MAPK inhibitors include SB203580, BIRB796 (Doramapimod), VX702, SB202190, LY2228820, VX745, Vinorelbine (Navelbine), PH797804, pamapimod, CMPD-1, EO1428, JX401, ML3403, RWJ67657, SB239063, SCIO469 hydrochoride, SKF86002 dihydrochloride, SX011, and TAK715.

In one embodiment, the method of the invention comprises administering a p38 MAPK inhibitor to a specific cell or tissue type of the subject. For example, in one embodiment, the method comprises administering an effective amount of a p38 MAPK inhibitor to a myeloid cell of a subject. In another embodiment, the method comprises administering an effective amount of a p38 MAPK inhibitor to a microglial cell of a subject. In another embodiment, the method comprises administering an effective amount of a p38 MAPK inhibitor to a macrophage of a subject. In another embodiment, the method comprises administering an effective amount of a p38 MAPK inhibitor to a dendritic cell of a subject. In another embodiment, the method comprises administering an effective amount of a p38 MAPK inhibitor to a neutrophil of a subject.

### Methods of Delivering a p38 MAPK Inhibitor to a Cell

The present invention comprises a method for treating or preventing an autoimmune disorder (e.g. MS), a neurodegenerative disorder, or neuroinflammation in a subject, said method comprising administering a therapeutic amount of a p38 MAPK inhibitor.

Isolated nucleic acid-based p38 MAPK inhibitors can be delivered to a cell in vitro or in vivo using viral vectors comprising one or more isolated p38 MAPK inhibitor sequences. Generally, the nucleic acid sequence has been incorporated into the genome of the viral vector. The viral vector comprising an isolated p38 MAPK inhibitor nucleic acid described herein can be contacted with a cell in vitro or in vivo and infection can occur. The cell can then be used experimentally to study, for example, the effect of an isolated p38 MAPK inhibitor in vitro, or the cells can be implanted into a subject for therapeutic use. The cell can be migratory, such as a hematopoietic cell, or non-migratory. The cell can be present in a biological sample obtained from the subject (e.g., blood, bone marrow, tissue, fluids, organs, etc.) and used in the treatment of disease, or can be obtained from cell culture.

After contact with the viral vector comprising an isolated p38 MAPK inhibitor nucleic acid sequence, the sample can be returned to the subject or re-administered to a culture of subject cells according to methods known to those practiced in the art. In the case of delivery to a subject or experimental animal model (e.g., rat, mouse, monkey, chimpanzee), such a treatment procedure is sometimes referred to as ex vivo treatment or therapy. Frequently, the cell is removed from the subject or animal and returned to the subject or animal once contacted with the viral vector comprising the isolated inhibitor nucleic acid of the present invention. Ex vivo gene therapy has been described, for example, in Kasid et al., Proc. Natl. Acad. Sci. USA 87:473 (1990); Rosenberg et al, New Engl. J Med. 323:570 (1990); Williams et al., Nature 310476 (1984); Dick et al., Cell 42:71 (1985); Keller et al., Nature 318:149 (1985) and Anderson et al., U.S. Pat. No. 5,399,346 (1994).

Where a cell is contacted in vitro, the cell incorporating the viral vector comprising an isolated p38 MAPK inhibitor nucleic acid can be implanted into a subject or experimental animal model for delivery or used in in vitro experimentation to study cellular events mediated by p38 MAPK inhibitor activity.

Various viral vectors can be used to introduce an isolated p38 MAPK inhibitor nucleic acid into mammalian cells. Viral vectors include retrovirus, adenovirus, parvovirus (e.g., adeno-associated viruses), coronavirus, negative-strand RNA viruses such as orthomyxovirus (e.g., influenza virus), rhabdovirus (e.g., rabies and vesicular stomatitis virus), paramyxovirus (e.g. measles and Sendai), positive-strand RNA viruses such as picornavirus and alphavirus, and double stranded DNA viruses including adenovirus, herpesvirus (e.g., herpes simplex virus types 1 and 2, Epstein-Barr virus, cytomegalovirus), and poxvirus (e.g. vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D-type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus, lentiviruses and baculoviruses.

In addition, an engineered viral vector can be used to deliver an isolated p38 MAPK inhibitor nucleic acid of the present invention. These vectors provide a means to introduce nucleic acids into cycling and quiescent cells, and have been modified to reduce cytotoxicity and to improve genetic stability. The preparation and use of engineered Herpes simplex virus type 1 (Krisky et al., 1997, Gene Therapy 4:1120-1125), adenoviral (Amalfitanl et al., 1998, Journal of Virology 72:926-933) attenuated lentiviral (Zufferey et al., 1997, Nature Biotechnology 15:871-875) and adenoviral/retroviral chimeric (Feng et al., 1997, Nature Biotechnology 15:866-870) vectors are known to the skilled artisan. In addition to delivery through the use of vectors, an isolated p38 MAPK inhibitor nucleic acid can be delivered to cells without vectors, e.g. as “naked” nucleic acid delivery using methods known to those of skill in the art. See, for example, U.S. Pat. Nos. 5,350,674 and 5,585,362.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York), and in Ausubel et al., 2002, Current Protocols in Molecular Biology, John Wiley & Sons, NY).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art.

Various forms of an isolated p38 MAPK inhibitor nucleic acid, as described herein, can be administered or delivered to a mammalian cell (e.g., by virus, direct injection, or liposomes, or by any other suitable methods known in the art or later developed). The methods of delivery can be modified to target certain cells, and in particular, cell surface receptor molecules. As an example, the use of cationic lipids as a carrier for nucleic acid constructs provides an efficient means of delivering the isolated TLR agonist nucleic acid of the present invention.

Various formulations of cationic lipids have been used to deliver nucleic acids to cells (WO 91/17424; WO 91/16024; U.S. Pat. Nos. 4,897,355; 4,946,787; 5,049,386; and 5,208,036). Cationic lipids have also been used to introduce foreign polynucleotides into frog and rat cells in vivo (Holt et al., Neuron 4:203-214 (1990); Hazinski et al., Am. J. Respr. Cell. Mol. Biol. 4:206-209 (1991)). Therefore, cationic lipids may be used, generally, as pharmaceutical carriers to provide biologically active substances (for example, see WO 91/17424; WO 91/16024; and WO 93/03709). Thus, cationic liposomes can provide an efficient carrier for the introduction of polynucleotides into a cell.

Further, liposomes can be used as carriers to deliver a nucleic acid to a cell, tissue or organ. Liposomes comprising neutral or anionic lipids do not generally fuse with the target cell surface, but are taken up phagocytically, and the polynucleotides are subsequently subjected to the degradative enzymes of the lysosomal compartment (Straubinger et al., 1983, Methods Enzymol. 101:512-527; Mannino et al., 1988, Biotechniques 6:682-690). Methods of delivering a nucleic acid to a cell, tissue or organism, including liposome-mediated delivery, are known in the art and are described in, for example, Felgner (Gene Transfer and Expression Protocols Vol. 7, Murray, E. J. Ed., Humana Press, New Jersey, (1991)).

In other related aspects, the invention includes an isolated p38 MAPK inhibitor nucleic acid operably linked to a nucleic acid comprising a promoter/regulatory sequence such that the nucleic acid is preferably capable of delivering an isolated p38 MAPK inhibitor nucleic acid. Thus, the invention encompasses expression vectors and methods for the introduction of an isolated p38 MAPK inhibitor nucleic acid into or to cells.

Such delivery can be accomplished by generating a plasmid, viral, or other type of vector comprising an isolated p38 MAPK inhibitor nucleic acid operably linked to a promoter/regulatory sequence which serves to introduce the p38 MAPK inhibitor into cells in which the vector is introduced. Many promoter/regulatory sequences useful for the methods of the present invention are available in the art and include, but are not limited to, for example, the cytomegalovirus immediate early promoter enhancer sequence, the SV40 early promoter, as well as the Rous sarcoma virus promoter, and the like. Moreover, inducible and tissue specific expression of an isolated p38 MAPK inhibitor nucleic acid may be accomplished by placing an isolated p38 MAPK inhibitor nucleic acid, with or without a tag, under the control of an inducible or tissue specific promoter/regulatory sequence. Examples of tissue specific or inducible promoter/regulatory sequences which are useful for his purpose include, but are not limited to the MMTV LTR inducible promoter, and the SV40 late enhancer/promoter. In addition, promoters which are well known in the art which are induced in response to inducing agents such as metals, glucocorticoids, and the like, are also contemplated in the invention. Thus, it will be appreciated that the invention includes the use of any promoter/regulatory sequence, which is either known or unknown, and which is capable of driving expression of the desired protein operably linked thereto.

Selection of any particular plasmid vector or other vector is not a limiting factor in this invention and a wide plethora of vectors are well-known in the art. Further, it is well within the skill of the artisan to choose particular promoter/regulatory sequences and operably link those promoter/regulatory sequences to a DNA sequence encoding a desired polypeptide. Such technology is well known in the art and is described, for example, in Sambrook et al. (2001, Molecular Cloning: A Laboratory Manual, Cold Spring Harbor Laboratory, New York), and in Ausubel et al., 2002, Current Protocols in Molecular Biology, John Wiley & Sons, NY and elsewhere herein.

A p38 MAPK inhibitor that is a peptide, polypeptide or protein can be supplied to cells. Protein can be produced by expression of the cDNA sequence in bacteria, for example, using known expression vectors. Alternatively, a p38 MAPK inhibitor polypeptide can be extracted from p38 MAPK inhibitor-producing mammalian cells. In addition, the techniques of synthetic chemistry can be employed to synthesize p38 MAPK inhibitor protein. Any of such techniques can provide the preparation of the present invention which comprises the p38 MAPK inhibitor protein. The preparation is substantially free of other human proteins. This is most readily accomplished by synthesis in a microorganism or in vitro.

Active p38 MAPK inhibitor protein can be introduced into cells by microinjection or by use of liposomes, for example. Alternatively, some active molecules may be taken up by cells, actively or by diffusion. Modified polypeptides having substantially similar function are also used for peptide therapy.

Combined with certain formulations, a peptide or protein, such as an antibody, which has p38 MAPK inhibitor activity can be effective intracellular agents if provided as a fusion protein along with a second peptide that promotes “transcytosis”, e.g., uptake of the peptide by cells. To illustrate, an antibody that inhibits p38 MAPK activity can be provided as part of a fusion polypeptide with all or a fragment of the N-terminal domain of the HIV protein Tat, e.g., residues 1-72 of Tat or a smaller fragment thereof which can promote transcytosis. In other embodiments, an antibody that inhibits p38 MAPK activity can be provided a fusion polypeptide with all or a portion of the antenopedia III protein.

To further illustrate, a p38 MAPK inhibitor peptide, polypeptide, or protein (or peptidomimetic) can be provided as a chimeric peptide which includes a heterologous peptide sequence (“internalizing peptide”) which drives the translocation of an extracellular form of a peptide with p38 MAPK inhibitory activity across a cell membrane in order to facilitate intracellular localization of the peptide with p38 MAPK inhibitory activity. In this regard, the therapeutic peptide with p38 MAPK inhibitory activity binding sequence is one which is active intracellularly. The internalizing peptide, by itself, is capable of crossing a cellular membrane by, e.g., transcytosis, at a relatively high rate. The internalizing peptide is conjugated, e.g., as a fusion protein, to the peptide or protein with p38 MAPK inhibitory activity. The resulting chimeric peptide is transported into cells at a higher rate relative to the activator polypeptide alone to thereby provide a means for enhancing its introduction into cells to which it is applied.

### Pharmaceutical Compositions and Therapies

Administration of a p38 MAPK inhibitor comprising one or more peptides, small molecules, antisense nucleic acids, or antibodies of the invention in a method of treatment may be achieved in a number of different ways, using methods known in the art. Such methods include, but are not limited to, providing an exogenous peptide inhibitor, small molecule, or antibody to a subject or expressing a recombinant peptide inhibitor, small molecule, soluble receptor, or antibody expression cassette.

The therapeutic and prophylactic methods of the invention thus encompass the use of pharmaceutical compositions comprising p38 MAPK inhibitor peptide, fusion protein, small molecule, or antibody of the invention and/or an isolated nucleic acid encoding a p38 MAPK inhibitory peptide, fusion protein small molecule, or antibody of the invention to practice the methods of the invention. The pharmaceutical compositions useful for practicing the invention may be administered to deliver a dose of 1 ng/kg/day to 100 mg/kg/day. In one embodiment, the invention envisions administration of a dose that results in a concentration of the compound of the present invention between 1 μM and 10 μM in a mammal, preferably a human.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, dogs, cats, rat, and mice.

Typically, dosages which may be administered in a method of the invention to an animal, preferably a human, range in amount from 0.5 μg to about 50 mg per kilogram of body weight of the animal. While the precise dosage administered will vary depending upon any number of factors, including but not limited to, the type of animal and type of disease state being treated, the age of the animal and the route of administration, the dosage of the compound will preferably vary from about 1 μg to about 10 mg per kilogram of body weight of the animal. More preferably, the dosage will vary from about 3 μg to about 1 mg per kilogram of body weight of the animal.

Pharmaceutical compositions that are useful in the methods of the invention may be prepared, packaged, or sold in formulations suitable for oral, parenteral, topical, buccal, or another route of administration. Other contemplated formulations include projected nanoparticles, liposomal preparations, resealed erythrocytes containing the active ingredient, and immunologically-based formulations.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

In one embodiment, the compositions of the invention are formulated using one or more pharmaceutically acceptable excipients or carriers. In one embodiment, the pharmaceutical compositions of the invention comprise a therapeutically effective amount of a compound or conjugate of the invention and a pharmaceutically acceptable carrier. Pharmaceutically acceptable carriers that are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent that delays absorption, for example, aluminum monostearate or gelatin. In one embodiment, the pharmaceutically acceptable carrier is not DMSO alone.

Formulations may be employed in admixtures with conventional excipients, i.e., pharmaceutically acceptable organic or inorganic carrier substances suitable for oral, vaginal, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition preferably includes an anti-oxidant and a chelating agent that inhibits the degradation of the compound. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition that may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

Controlled- or sustained-release formulations of a composition of the invention may be made using conventional technology, in addition to the disclosure set forth elsewhere herein. In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the compositions of the invention.

Controlled-release of an active ingredient can be stimulated by various inducers, for example pH, temperature, enzymes, water, or other physiological conditions or compounds. The term “controlled-release component” in the context of the present invention is defined herein as a compound or compounds, including, but not limited to, polymers, polymer matrices, gels, permeable membranes, liposomes, nanoparticles, or microspheres or a combination thereof that facilitates the controlled-release of the active ingredient.

### Administration/Dosing

The regimen of administration may affect what constitutes an effective amount. The therapeutic formulations may be administered to the subject either prior to or after a diagnosis of disease. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a subject, preferably a mammal, more preferably a human, may be carried out using known procedures, at dosages and for periods of time effective to prevent or treat disease. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, weight, condition, general health and prior medical history of the subject being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc. The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a subject.

In one embodiment, the compositions of the invention are administered to the subject in dosages that range from one to five times per day or more. In another embodiment, the compositions of the invention are administered to the subject in range of dosages that include, but are not limited to, once every day, every two, days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any subject will be determined by the attending physical taking all other factors about the subject into account.

Compounds of the invention for administration may be in the range of from about 1 mg to about 10,000 mg, about 20 mg to about 9,500 mg, about 40 mg to about 9,000 mg, about 75 mg to about 8,500 mg, about 150 mg to about 7,500 mg, about 200 mg to about 7,000 mg, about 3050 mg to about 6,000 mg, about 500 mg to about 5,000 mg, about 750 mg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments therebetween.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., a drug used for treating the same or another disease as that treated by the compositions of the invention) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the present invention is directed to a packaged pharmaceutical composition comprising a container holding a therapeutically effective amount of a composition of the invention, alone or in combination with a second pharmaceutical agent; and instructions for using the composition to treat, prevent, or reduce one or more symptoms of a disease in a subject.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating or preventing a disease in a subject.

### Routes of Administration

Routes of administration of any of the compositions of the invention include oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, intracranial, intracerebroventricular, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intrasternal injection, intratumoral, intracranial, intracerebroventricular, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

### Kits of the Invention

The invention also includes a kit comprising a p38 MAPK inhibitor and an instructional material that describes, for instance, administering the p38 MAPK inhibitor to a subject as a prophylactic or therapeutic treatment or a non-treatment use as described elsewhere herein. In an embodiment, the kit further comprises a (preferably sterile) pharmaceutically acceptable carrier suitable for dissolving or suspending the therapeutic composition, comprising a p38 MAPK inhibitor, for instance, prior to administering the molecule to a subject. Optionally, the kit comprises an applicator for administering the inhibitor.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### p38 MAPK as a Female-Specific Druggable Target in Autoimmune Disease of the CNS

The data presented herein further investigates the role of p38 MAPK in mediating the inflammatory responses during EAE, the principle autoimmune model of multiple sclerosis. Previous studies have shown that p38 MAPK activation is required for the development and progression of both chronic and relapsing-remitting forms EAE. Furthermore, it was shown that regulation of p38 MAPK activity specifically in T cells is sufficient to modulate EAE severity (Noubade et al., 2011, Blood; 118(12): 3290-3300, which is incorporated herein by reference). The present data demonstrates a gender-specific role of p38 MAPK.

The materials and methods used in the following experiments are now described.

Mice

C57BL/6J (B6) and B10.BR-H2^{k} H2-T18/SgSnJ (B10.BR) mice were purchased from The Jackson Laboratory. MKK6 transgenic (Rincon et al., 1998, EMBO J., 17(10):2817-2829) and do-p38 transgenic (Diehl et al., 2000, J Exp Med, 191(2):321-334) mice have been described.

Induction and Evaluation of EAE

EAE was induced in female C57BL/6J mice as described previously (Noubade et al., 2007, J Clin Invest., 117(11):3507-3518). Mice were injected subcutaneously with an emulsion containing 200 μg of MOG₃₅₋₅₅ peptide (MEVGWYRSPFSRVVHLYRNGK (SEQ ID NO: 1) and complete Freund adjuvant (CFA; Sigma-Aldrich) supplemented with 200 μg of Mycobacterium tuberculosis H37RA (Difco Laboratories) in the posterior right and left flank; 1 week later, all mice were similarly injected at 2 sites on the right and left flank anterior of the initial injection sites (2×MOG₃₅₋₅₅+CFA). Mice received 5 mg/kg/d SB203580 dihydrochloride (Tocris) by IP injection in a total volume of 200 μL or an equal volume of carrier every day from the day of immunization.

EAE was induced in B10.BR, MKK6-Tg, and dn-p38-Tg mice by immunizing with a single injection of 200 μg of MOG₉₇₋₁₁₄ (TCFFRDHSYQEEAAVELK (SEQ ID NO: 2)) or PLP₁₈₀₋₂₀₉ (SKTSASIGSLCADARMYGVL (SEQ ID NO: 3)) in CFA. Immediately thereafter, each animal received 200 ng of PTX (List Biologic Laboratories) by IV injection. Mice were scored daily starting at day 10 after injection as previously described (Noubade et al., 2007, J Clin Invest., 117(11):3507-3518). Clinical quantitative trait variables were generated as previously described (Butterfield et al., 1998, J Immunol., 161(4):1860-1867).

Cell Preparation and Culture Conditions

Total CD4 T cells were isolated from spleen and lymph nodes as previously described (Noubade et al., 2007, J Clin Invest., 117(11):3507-3518), by negative selection for CD8-, MHC class II-, NK1.1- and CD11b-positive cells using magnetic beads from QIAGEN. For FACS sorting, negatively selected CD4 T cells were stained with anti-TCRβ-allophycocyanin and anti-CD4-Texas Red (Invitrogen), and sorted using a FACSAria cell sorting system (BD Biosciences). Th17 CD4 T cells were generated by activating total CD4 T cells (1×10⁶ cells/mL) in RPMI containing 10% FBS (Hyclone), with plate bound anti-CD3 (5 μg/mL) and soluble anti-CD28 (1 μg/mL) mAbs from BD Pharmingen in the presence of 1 ng/mL TGFβ (PeproTech Inc), 30 ng/mL IL-6 (R&D Systems), 10 μg/mL anti-IFNγ, and 10 μg/mL anti-IL-4 mAbs. For the FACS-sorted cells, the Th17 conditions included activating cells with plate-bound anti-CD3 (5 μg/mL) and soluble anti-CD28 (1 μg/mL) mAbs in the presence of 1 ng/mL TGFβ and 100 ng/mL IL-6 and no neutralizing mAbs. Depending on the experiment, cells were treated the p38 MAPK inhibitors SB203580 (Calbiochem) or BIRB796 (Axon Medchem) or the MNK inhibitor CGP57380 (Sigma-Aldrich). Cells were incubated at 37° C. and 5% CO₂ for the desired lengths of time as described in the figure legends.

Cytokine Quantification

For the detection of cytokines in the cell-culture supernatants, ELISAs were performed as described previously (Noubade et al., 2007, J Clin Invest., 117(11):3507-3518), using the primary mAbs: anti-IFNγ, anti-IL-2, and anti-IL-17A and their corresponding biotinylated mAbs (BD Pharmingen). Other ELISA reagents included: HRP-conjugated avidin D (Vector Laboratories) and TMB microwell peroxidase substrate and stop solution (Kirkegaard & Perry Laboratories). rIFNγ, rIL-17A, and rIL-2 (R&D Systems) were used as standards.

Flow Cytometry

For intracellular cytokine staining, either FACS-sorted or total CD4 T cells polarized to Th17 cells in the absence or the presence of 5 μM SB203580 were stimulated with 5 ng/mL PMA, 250 ng/mL ionomycin, and 2 μM monensin (Sigma-Aldrich) for the last 4 hours of culture. Cells harvested at the end of the incubation were first stained with LIVE/DEAD fixable stain (Invitrogen) and anti-CD4-Texas Red. Cells were then fixed with 4% paraformaldehyde (Sigma-Aldrich), permeabilized with buffer containing 0.2% saponin and stained with anti-IL-17A-PE (BD Pharmingen) and anti-IFNγ-Alexa 647 (BD Pharmingen). Cells were collected using a LSR II cytometer (BD Biosciences) and analyzed using FlowJo software (TreeStar Inc).

Quantitative Real-Time PCR

Total RNA was extracted from CD4 T cells using RNeasy RNA isolation reagent (QIAGEN) as recommended by the manufacturer. The generated cDNA was used in quantitative real-time PCR using the assay-on-demand (AOD) TaqMan probe and primers for IL-17A and β2-microglobulin (Applied Biosystems). β2-microglobulin was used as a reference gene and relative mRNA levels were calculated using the comparative C_{T} method.

CNS-Infiltrating Mononuclear Cell Isolation

Animals were perfused with saline and brains and spinal cords removed. A single-cell suspension was obtained and passed through a 70-μm strainer. Mononuclear cells were obtained by Percoll gradient (37%/70%) centrifugation and collected from the interphase. Cells were washed and stimulated for 4 hours with PMA/ionomycin in the presence of brefeldin A (Golgi Plug; BD Biosciences). Cell were labeled with LIVE/DEAD UV-Blue dye (Invitrogen) followed by surface staining (CD45 from Invitrogen and CD4, CD8 and TCRβ from BD Biosciences). Afterward, cells were fixed, permeabilized, and stained for intracellular IL-17A (BD Biosciences) and IFNγ (Invitrogen).

The results of the experiments are now described.

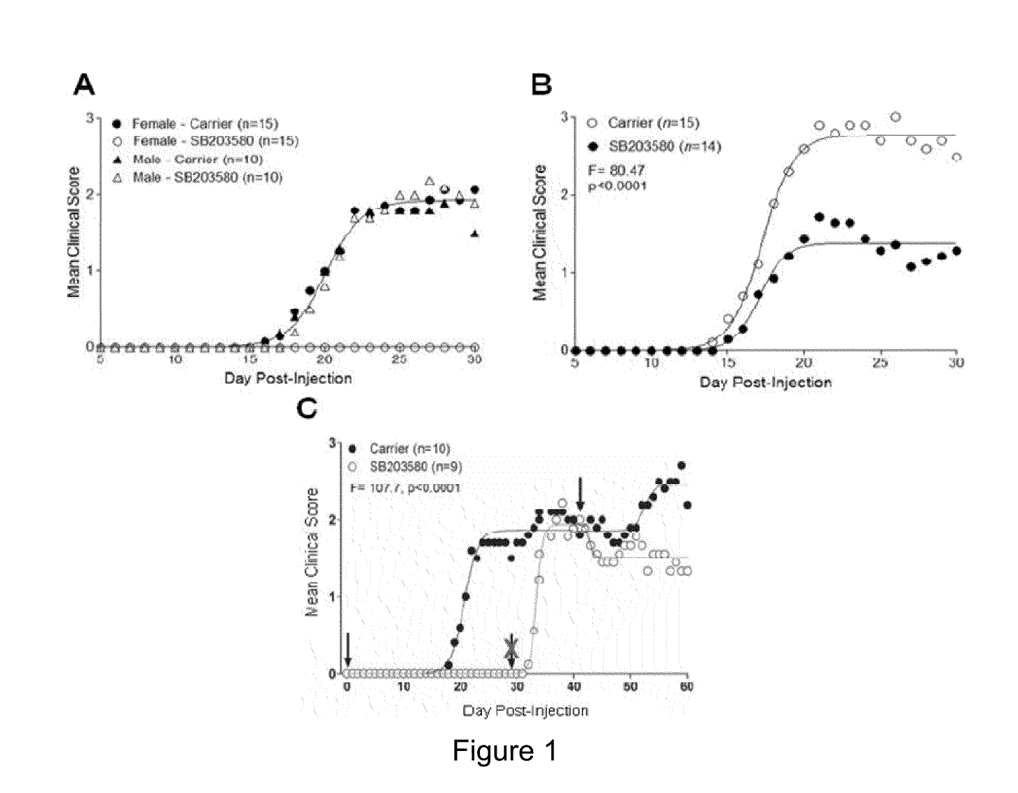

Age-matched male and female C57BL/6J (B6) mice were treated with the p38 MAPK inhibitor SB203580 (SB) starting at the time of immunization for the induction of EAE with myelin oligodendrocyte glycoprotein peptide 35-55 (MOG35-55) and complete Freund's adjuvant (CFA). Clinical EAE course in male and female B6 mice immunized with 2×MOG₃₅₋₅₅-CFA treated daily with either carrier or SB starting on D0. Treatment with SB fully prevented disease in females (FIG. 1A). Strikingly, male mice were completely unresponsive to SB treatment (FIG. 1A). In separate experiments, Clinical EAE course in female B6 mice immunized with 2×MOG₃₅₋₅₅-CFA and randomly selected for daily treatment with either carrier or SB upon reaching a clinical score≧1. It was observed that disease progression could also be halted in female mice if the inhibitor was administered at the first onset of clinical signs (FIG. 1B). To show that EAE suppression is dependent on active inhibition of p38 MAPK, SB treatment was stopped after 30 days. Within 2-3 days of discontinuing SB treatment, clinical signs of EAE appeared, reaching equivalent severity to those seen in carrier-treated mice (FIG. 1C). Readministration of SB led to a modest reduction and stabilization of their disease severity which was not seen in carrier-treated controls (FIG. 1C). These data suggest that: 1) p38 activity is required for the development and progression of EAE; and 2) sex-specific factors contribute to SB-mediated prevention of EAE.

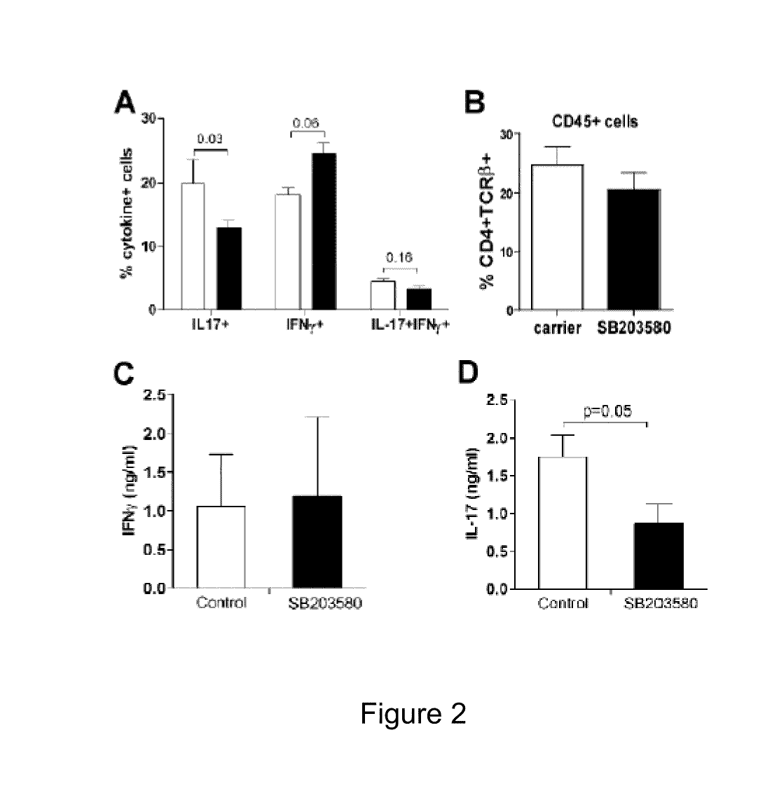

To examine the mechanism of EAE prevention by SB, the T_{H}1 and T_{H}17 effector subsets were analyzed in treated female mice. Female B6 mice were immunized for EAE as detailed above and treated daily with carrier or SB. On D30 post-immunization, CNS (brain and spinal cord) cells were harvested and analyzed by intracellular staining and flow cytometry. Surprisingly, it was found that SB treatment specifically inhibited IL-17 production by T_{H}17 cells, and did not suppress IFN-γ production by T_{H}1 cells, either in CNS-infiltrating cells during late-phase chronic disease (FIG. 2A). Interestingly, SB treatment did not decrease the percentage or number of infiltrating CD4+ T cells in the CNS (FIG. 2B). Draining lymph node (DLN) cells were collected on D20 and restimulated with MOG₃₅₋₅₅ for 3 days, and production of IFN-γ and IL-17 was assessed by ELISA. These studies again showed that SB treatment specifically inhibited IL-17 production but did not decrease or IFN-γ production in DLN on D20 post-immunization (FIG. 2C and FIG. 2D).

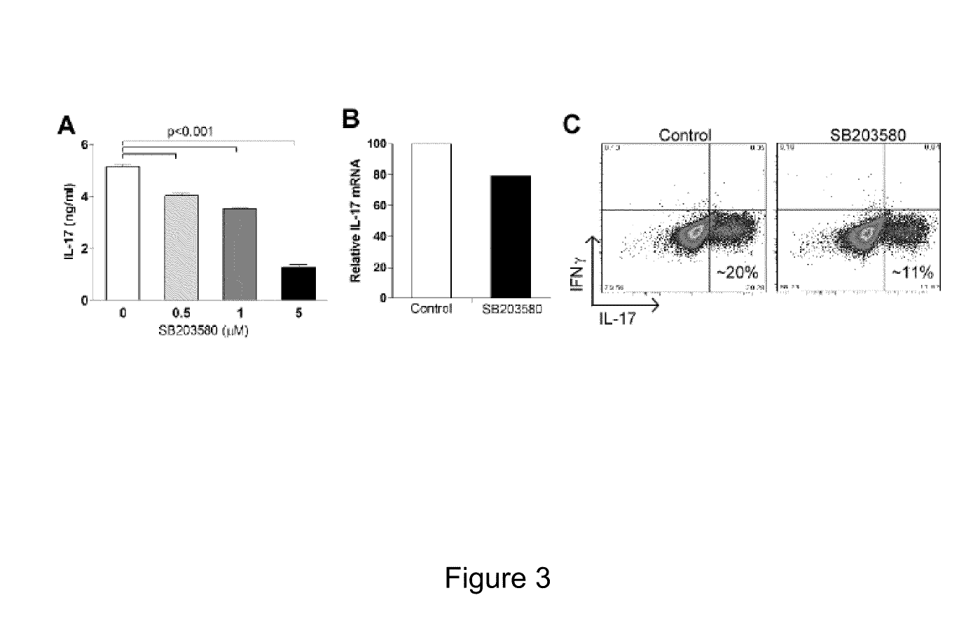

To further examine how p38 MAPK inhibition resulted in decreased IL-17 production, Naïve CD4+ T cells were purified by negative selection and cultured under T_{H}17 polarizing conditions for 3 days in the presence of SB20350 at a concentration of 5 μm (unless otherwise indicated). IL-17 production was analyzed by various methods including ELISA, qRT-PCR, and intracellular staining and flow cytometry. Consisted with other data presented herein, SB treatment during in vitro differentiation of TH17 cells also suppressed IL-17 production in a dose-dependent manner (FIG. 3A). Similar results were obtained with BIRB796, a different inhibitor of p38 MAPK. p38 MAPK regulates the production of many cytokines, such as IFN-γ, at the mRNA level, by acting on transcription factors (Rincon, et al., 1998, EMBO J 17:2817-2829). Unexpectedly, IL-17 mRNA levels were not significantly affected by SB treatment (FIG. 3B), whereas secreted and cellular protein levels were decreased (FIG. 3A and FIG. 3C), suggesting a novel mechanism involving post-transcriptional control of IL-17 production, rather than regulation at the mRNA level (Noubade et al., 2011, Blood; 118(12): 3290-3300).

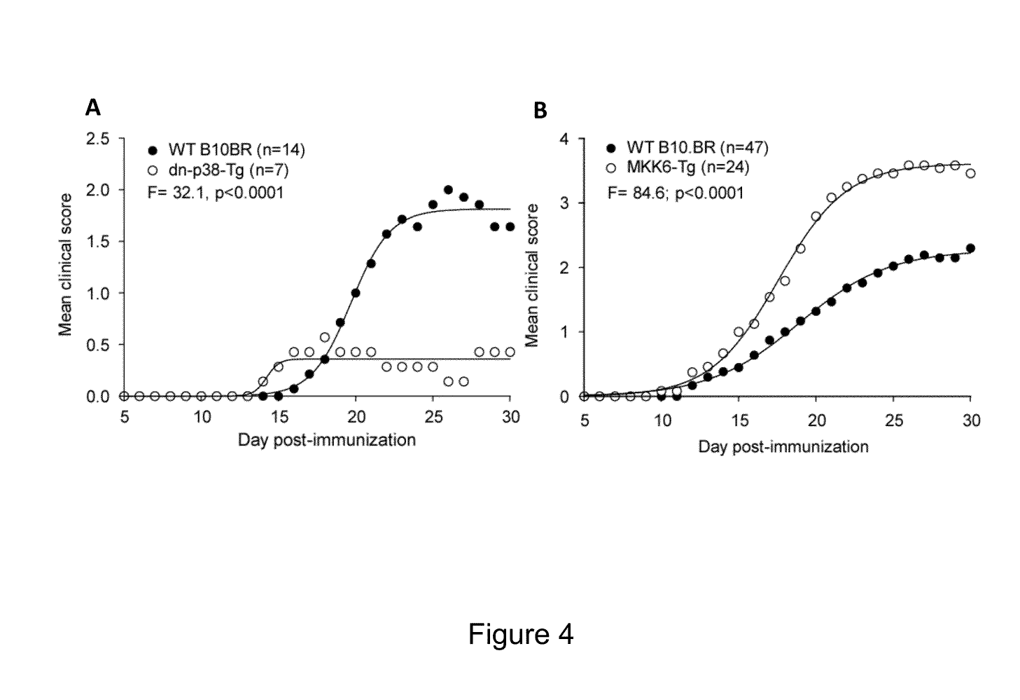

In order to directly assess the role of p38 MAPK in T cells during EAE, transgenic (Tg) B10.BR mice were utilized, where Tg mice expressed either a dominant negative form of p38 MAPK (dn-p38-Tg) or a constitutively active form of MKK6 (one of the kinases directly upstream of p38 MAPK; MKK6-Tg), under control of the distal lck promoter to drive expression specifically in T cells (Rincon and Davis, 2009, Immunol Rev 228:212-224). Wild-type (WT) and Tg mice were immunized for EAE, by immunizing with 1×PLP₁₈₀₋₂₀₉+CFA+PTX, and scored for clinical signs. Inhibition of p38 MAPK by do-p38 in T cells strikingly reduced disease severity and incidence (FIG. 4A), as well as in vitro IL-17 production. In contrast, constitutive activation of p38 MAPK by MKK6-Tg led to increased disease severity (FIG. 4B), and enhanced in vitro IL-17 secretion. These results suggest that manipulation of p38 MAPK activity in T cells alone is sufficient to alter EAE progression and susceptibility.

The results presented herein demonstrate the gender specificity of p38 MAPK antagonism on EAE severity. Further, it is shown that the protective effects of SB correlated with a decrease in the number of IL-17-producing T_{H}17 cells and SB treatment reduced IL-17 production in vitro.

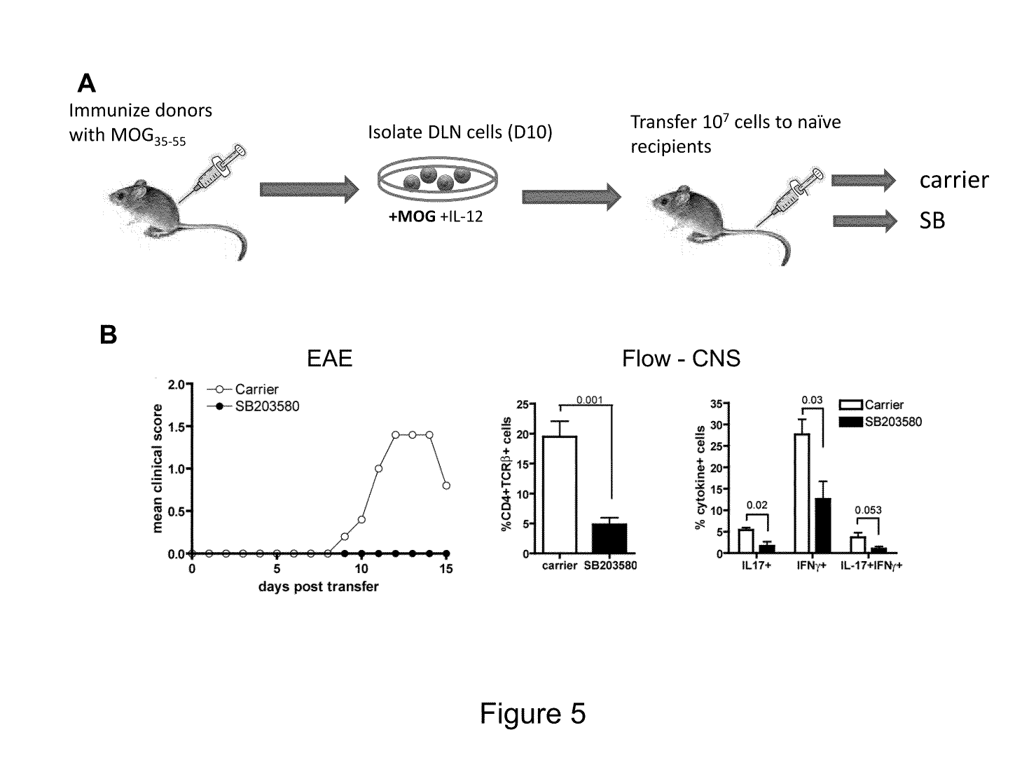

SB treatment completely inhibits EAE but only partially inhibits IL-17 production by TH17 cells. Based on this and the observation that IL-17 is not absolutely required for EAE progression (Haak, S et al., 2009, J Clin Invest 119:61-69), it is possible that SB prevents EAE by inhibiting production of additional key pathogenic factors. Further, it is shown that TH1-mediated passive EAE is prevented by inhibition of p38 MAPK (FIG. 5). In these experiments, donor animals were immunized with MOG35-55, DLN cells were isolated at D10 and cultured under polarizing conditions, and cells were transferred into naïve recipients. It was observed that SB treatment reduced EAE severity and decreased cytokine production. Thus, it is also likely that the treatment inhibits production of putative pathogenic factors (other than IFN-γ) by these cells. As such, complementary genomic and proteomic approaches were used to identify these factors, which in the future may provide novel druggable targets for treatment of autoimmune disease. Because such approaches typically yield a large number of potential candidates, a prioritizing/“funneling” scheme was constructed to rule out irrelevant hits in screens. In particular, the sexual dimorphism in response to SB serves as a useful discriminatory tool in this regard.

Using microarray technology, differences in gene expression were examined in the inflamed CNS (during peak EAE) between carrier- and SB-treated female and male B6 mice. While inhibition of p38 MAPK may alter the expression of many genes, it is predicted that only the genes that show differential expression in female but not in the non-responsive male mice are responsible for disease prevention by SB. Similar analyses were also performed using mice in which p38 MAPK activity is genetically manipulated in specific cell types. Microarray results were validated using qRT-PCR.

Importantly, data presented herein indicate that p38 MAPK can regulate the production of some pathogenic factors, such as IL-17, at the post-transcriptional level (FIG. 3). Thus, a complementary in vitro approach was employed where comparisons of gene and protein expression profiles (either secreted or cellular) of TH1/17 cells differentiated in the presence or absence of SB, or TH1/17 cells in which p38 MAPK activity is genetically manipulated were performed. Microarrays for gene expression analysis, and a mass-spectrometry approach (stable isotope labeling with amino acids in cell culture (SILAC)) for protein expression analysis were used. The SILAC approach allows for the quantification of relative abundance of proteins in two different samples (e.g. treated or untreated cells) (Ong, et al., 2003, Methods 29:124-130). Candidate pathogenic factors strongly altered in vitro by p38 MAPK inhibition were validated by immunoblot and RT-PCR analysis. Importantly, the differential expression of these factors in effector CD4+ T cells were then confirmed in vivo, in EAE-induced mice treated with SB or carrier, or in mice in which p38 MAPK activity is genetically manipulated, as above.

Since only female mice respond to SB (FIG. 1), it is possible that estrogen enhances this response and/or that testosterone blocks it. To examine this possibility, it was determined whether SB can prevent EAE in gonadectomized male and female mice, with or without hormone (estrogen, progesterone, or non-aromatizing testosterone, e.g. 1-T) replacement. Exogenous estrogen can be protective in MS and can modulate cytokine responses (Soldan, et al., 2003, J Immunol 171:6267-6274). Additionally, B6 mice deficient for each of the three estrogen-binding receptors, estrogen receptor (Esr)1, Esr2, and G-protein coupled estrogen receptor (Gper) were studied to test whether estrogen signaling is required for SB-mediated inhibition of EAE. Additionally, uterine weights of SB- or carrier-treated gonadectomized female mice were monitored to rule out any estrogenic effects of SB itself. In order to test whether the sexual dimorphism alters the response to SB by influencing effector cell generation in the periphery, effector cell function in the CNS, or both, passive EAE experiments were performed, by transferring male or female encephalitogenic CD4+ T cells into male or female recipients which were treated with carrier or SB.

Importantly, in all experiments, the CNS-directed immune responses were evaluated, to determine whether disease resistance correlates with diminished T_{H} responsiveness, as well as other immune parameters. The sexual dimorphism in response to SB allows the further understanding of the mechanism of drug action, e.g. by ruling out immune parameters that correlate with drug treatment in both males and females, and thus are not sufficient for disease prevention.

### Example 2

### Myeloid Specific Conditional Knock Out of p38alpha Induces Female-Specific Reduction of EAE Severity and Reduced Cytokine Production

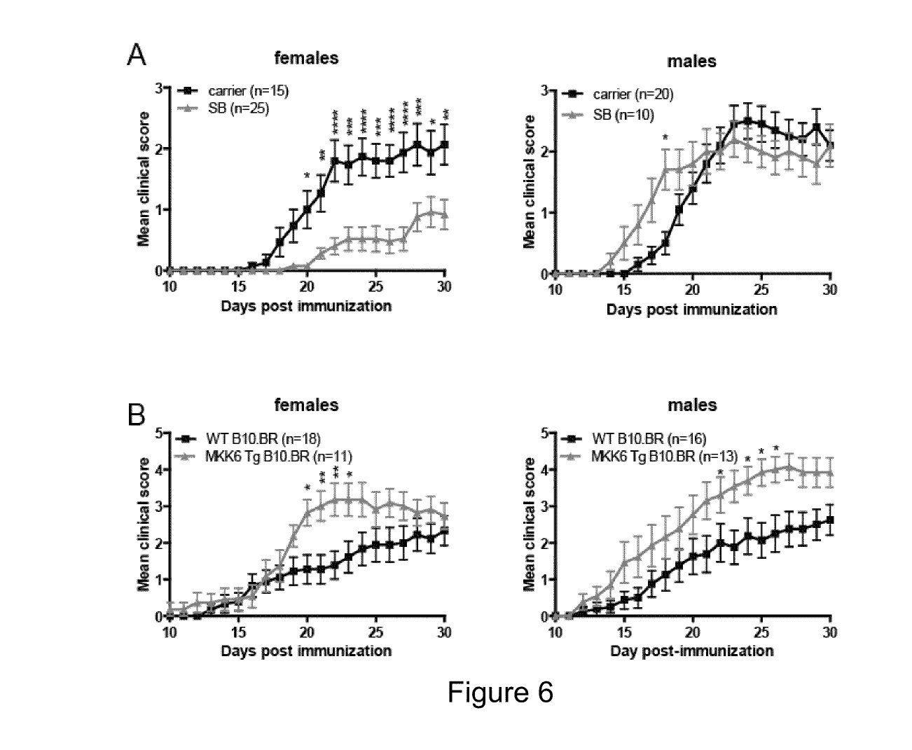

It is shown that pharmacological inhibition of p38 MAPK by SB203580 (SB) in female, but not male C57BL/6 (B6) mice ameliorated EAE (FIG. 6A). Further, as described elsewhere herein, it is indicated that genetic manipulation of p38 MAPK activity in T cells in B10.BR mice was sufficient to alter EAE progression.

Further experiments were performed where WT and Tg mice expressing constitutively active MKK6 (MKK6 Tg) were immunized using 1×CFA/MOG₇₉₋₉₆+PTX protocol and scored daily. These studies have now revealed that augmentation of p38 activity in T cells, in the form of MKK6 Tg B10.BR mice, enhanced disease in both males and females (FIG. 6B). Furthermore, inhibition of p38 activity by the expression of a dominant negative p38 MAPK allele inhibited disease in male and female mice. These results suggest that the sexual dimorphism in SB treatment response is: a) strain-specific (B6 vs. B10.BR); b) cell-type specific (SB may target cell types other than T cells); or c) bypassed by genetic manipulation of p38 activity.

To address these possibilities, B6 mice lacking p38alpha MAPK in T cells (p38CKOlck) or myeloid cells/macrophages (p38CKOLysM) were generated, since both cell types contribute vitally to EAE and MS pathogenesis, and p38alpha signaling in these cells typically plays a pro-inflammatory role (Rincon and Davis, 2009, Immunol Rev 228(1):212-24). In order to do this, B6 mice carrying a floxed p38alpha allele (p38^{f/f}; (Nishida, et al., 2004, Mol Cell Biol 24(24):10611-20)) were crossed to Lck-Cre transgenic mice (Hennet et al., 1995, Proc Natl Acad Sci USA 92(26):12070-4) (which express Cre in T cells) and to LysM-Cre mice (Clausen et al., 1999, Transgenic Res 8(4):265-77), which carry a Cre allele that was knocked into the LysM locus (expressing Cre in macrophages/microglia/myeloid cells).

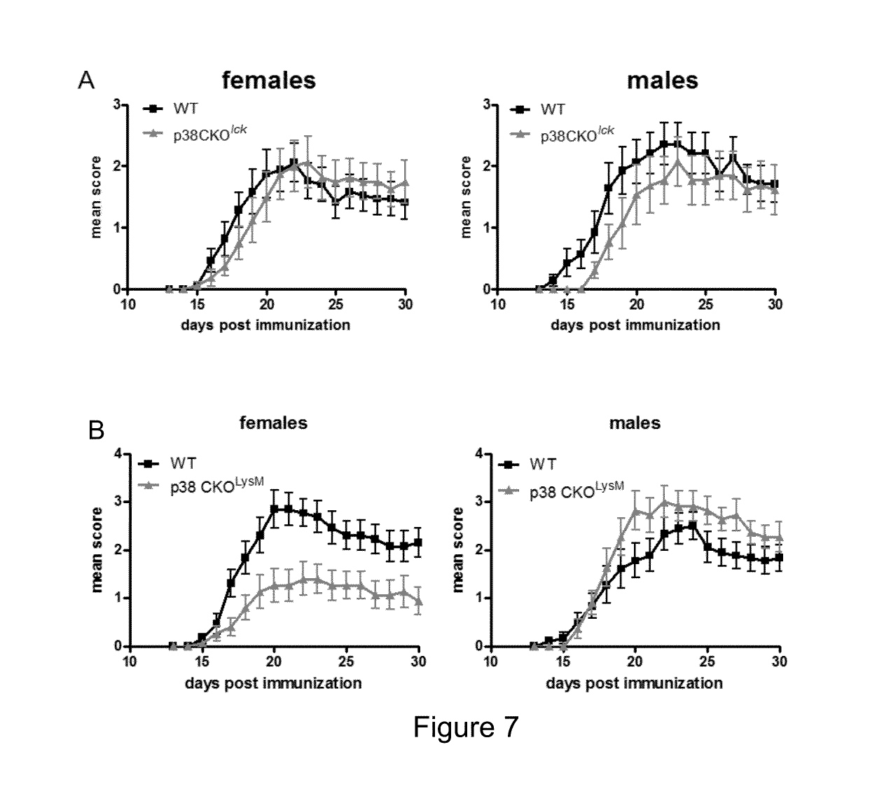

p38^{f/f} Lck-Cre Tg (p38CKO^{lck}) mice, p38^{f/f} LysM-Cre (p38CKO^{LysM}) mice, and littermate controls were immunized using 2×CFA/MOG₃₅₋₅₅ protocol and scored daily. It was observed that T cell-specific deletion of p38alpha in B6 mice had no significant effect on EAE in either sex (FIG. 7A), suggesting that in the B6 model, p38 signaling in T cells is dispensable for full EAE susceptibility, and does not control the sexual dimorphism in response to SB. In contrast, deletion of p38alpha in myeloid cells resulted in protection in females, but not males (FIG. 7B), suggesting that the sexual dimorphism in the EAE response to pharmacological blockade of p38 by SB occurs within the myeloid cell compartment. No significant effect of the LysM-Cre allele was observed in p38^{wt/wt} or p38^{f/wt} animals, ruling out non-specific effects of this allele on EAE.

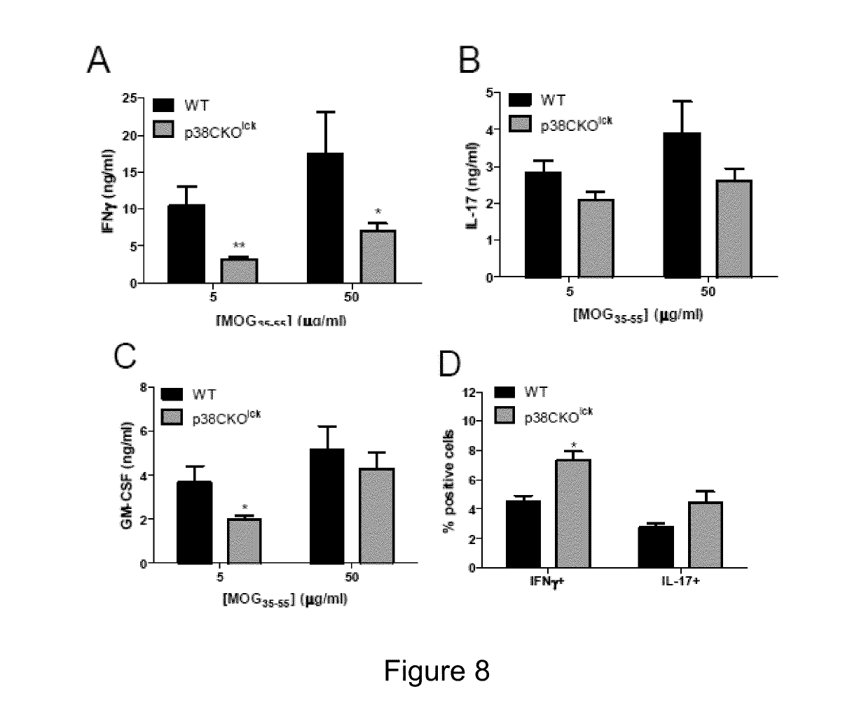

The peripheral T cell responses, as well as infiltration and activation of CNS infiltrating cells, were analyzed in p38CKO^{lck} and p38CKO^{LysM} mice. Female littermate p38^{f/f} (WT) and p38^{f/f} Lck-Cre Tg (p38CKO^{lck}) mice were immunized using 2×CFA/MOG₃₅₋₅₅ protocol. On D10, LN and spleen cells were isolated, restimulated with either 5 μg/ml or 50 μg/ml MOG₃₅₋₅₅, and production of IFNγ, IL-17, and GM-CSF was determined by ELISA. Lymphocytes from p38CKO^{lck} mice exhibited a modest reduction in cytokine production, particularly of IFNg (FIG. 8A-FIG. 8C). Furthermore, no differences were detected in the production of IFNg, IL-17, and GM-CSF by CNS-infiltrating T cells in p38CKO^{lck} mice during EAE. To examine the generation of Th1 or Th17 cells, LN and spleen cells were isolated on D10, and stimulated with PMA/Ionomycin in the presence of bredfeldin A for 4 hours. Cells were stained and processed by intracellular cytokine staining and flow cytometry. These experiments demonstrated that the generation of Th1 or Th17 cells per se was not reduced, (FIG. 8D). These results are consistent with the finding that deletion of p38alpha in T cells did not significantly affect EAE (FIG. 7A), and suggest that the in vivo effects of SB on T cells in B6 mice may be indirect.

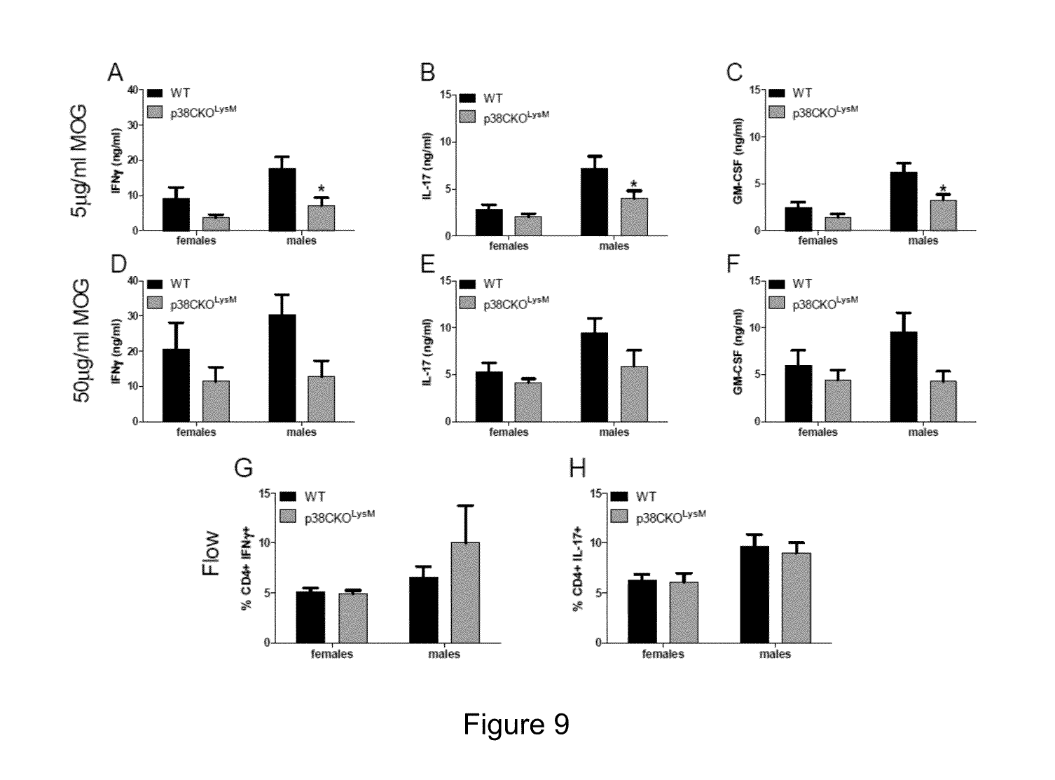

Similar experiments were performed on p38^{f/f} LysM-Cre Tg (p38CKO^{LysM}) male and female mice. Lymphocytes from p38CKO^{LysM} mice also exhibited a modest reduction in cytokine production in a recall response, compared to WT, but only at a lower MOG35-55 concentration, and this reached significance in only males, despite a similar trend in females (FIG. 9A-FIG. 9F). Furthermore, the generation of Th1 or Th17 cells, as measured by intracellular cytokine staining was not affected in either sex. These results suggest that p38 in myeloid cells is mostly dispensable for generating an effective peripheral T cell response in EAE. More importantly, no sex-by-genotype interaction was detected, indicating that the female-specific EAE response seen in disease course (FIG. 7) is not due to a defective peripheral T cell response.

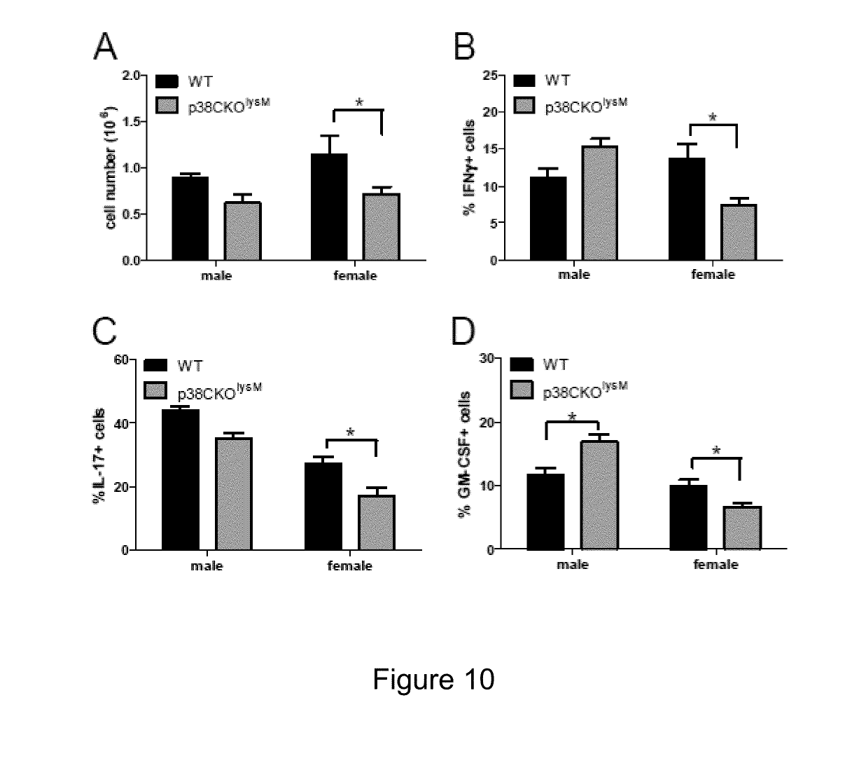

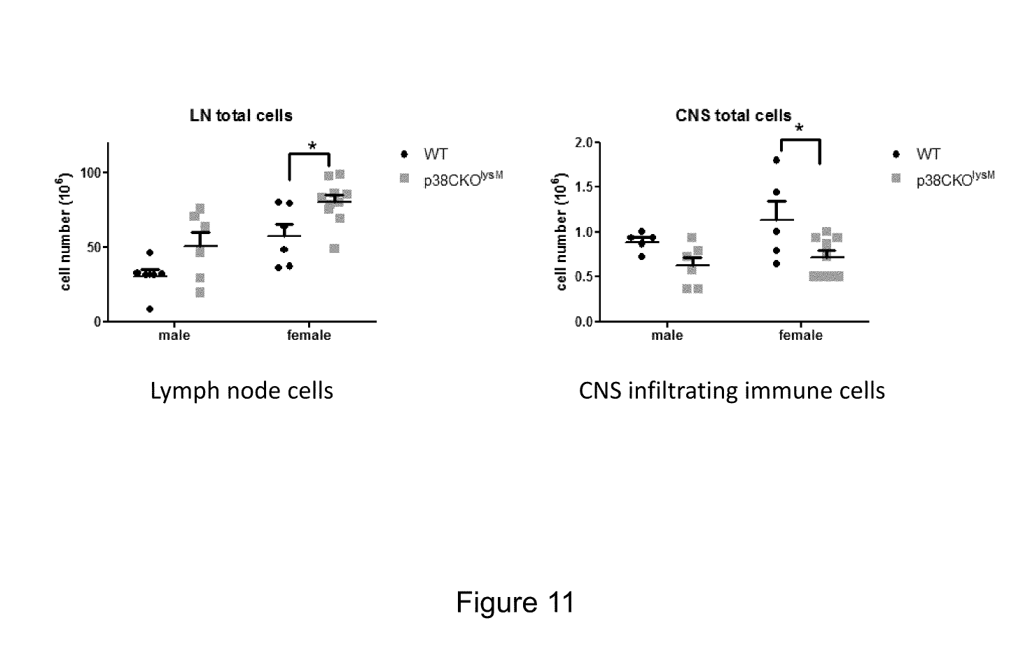

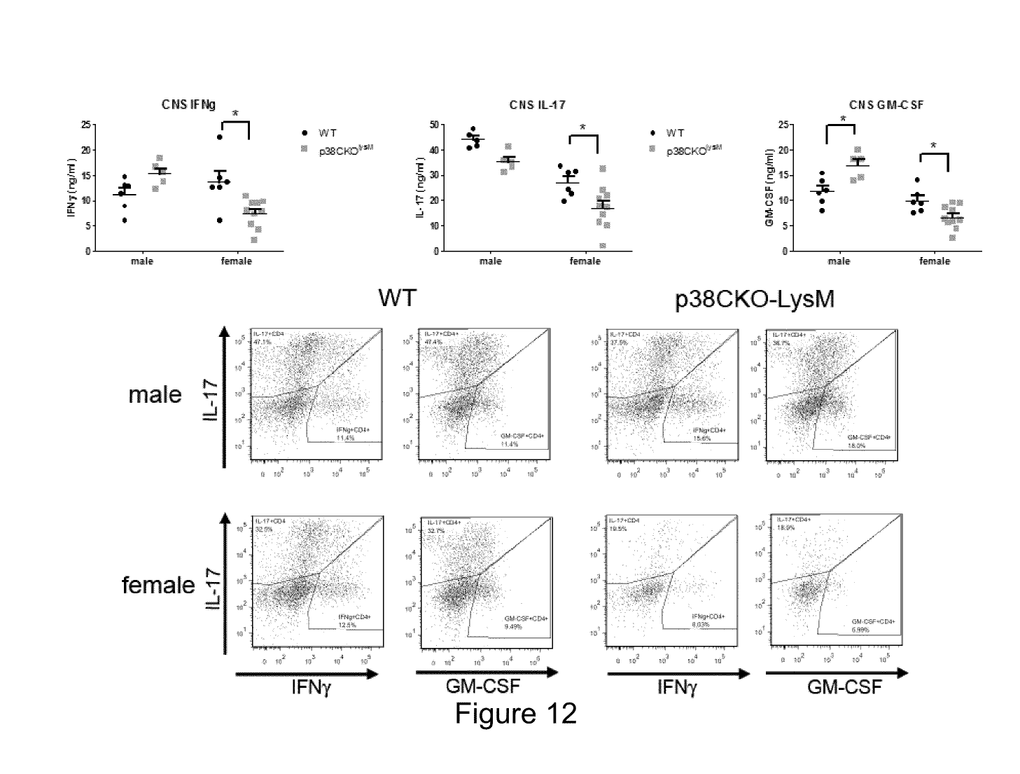

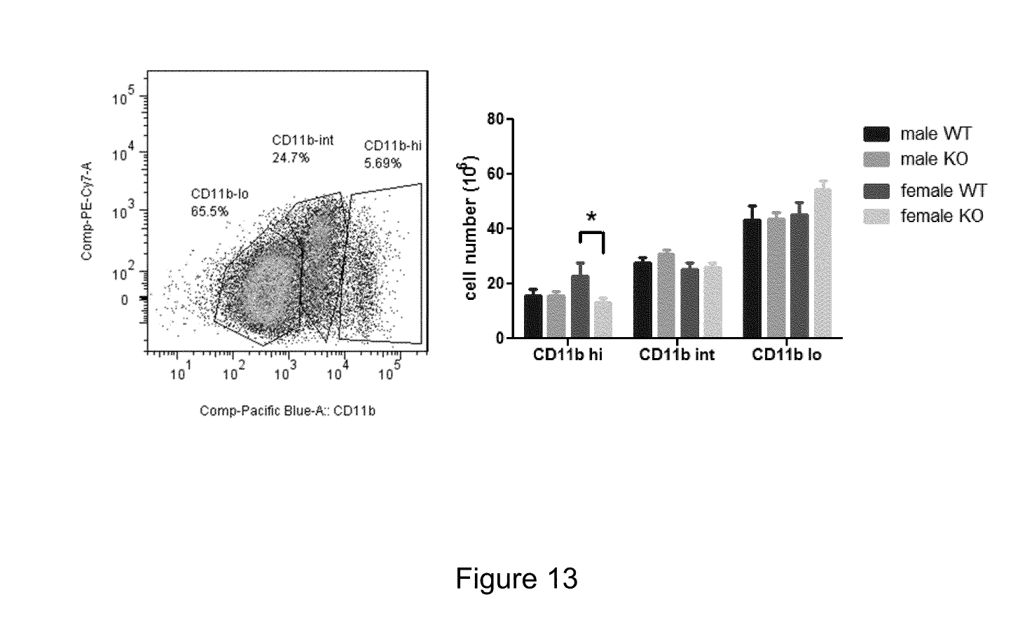

Experiments were performed where male and female littermate p38^{f/f} (WT) and p38^{f/f} LysM-Cre Tg (p38CKO^{LysM}) mice were immunized using 2×CFA/MOG₃₅₋₅₅ protocol. On D21, mononuclear cells were isolated from the CNS using a Percoll gradient, counted, and stimulated with MOG₃₅₋₅₅ for 4 hours in the presence of brefeldin A, then analyzed by ICCS and flow cytometry. It was seen that female mice lacking p38 in myeloid cells had increased lymph node cells and decreased CNS infiltrating immune cells at D21 (FIG. 10 and FIG. 11) Analysis of the inflammatory infiltrates in the CNS during peak EAE revealed that female, but not male mice lacking p38 in myeloid cells exhibited reduced infiltration into the CNS, as well as reduced production of IFNg, IL-17, and GM-CSF by CD4+ T cells compared with controls (FIG. 10 and FIG. 12). Importantly, there was a significant sex-by-genotype interaction for production IFNg and GM-CSF by CD4+ T cells, which correlates with the female-specific EAE response. Further analysis showed that myeloid specific deletion of p38 MAPK resulted in decreased CNS-infiltrating highly activated myeloid CD11b^{hi} cells at D21 post immunization (FIG. 13). Collectively, these results suggest ablation of p38alpha in myeloid cells dampens the female inflammatory response in the CNS, and not in the periphery.

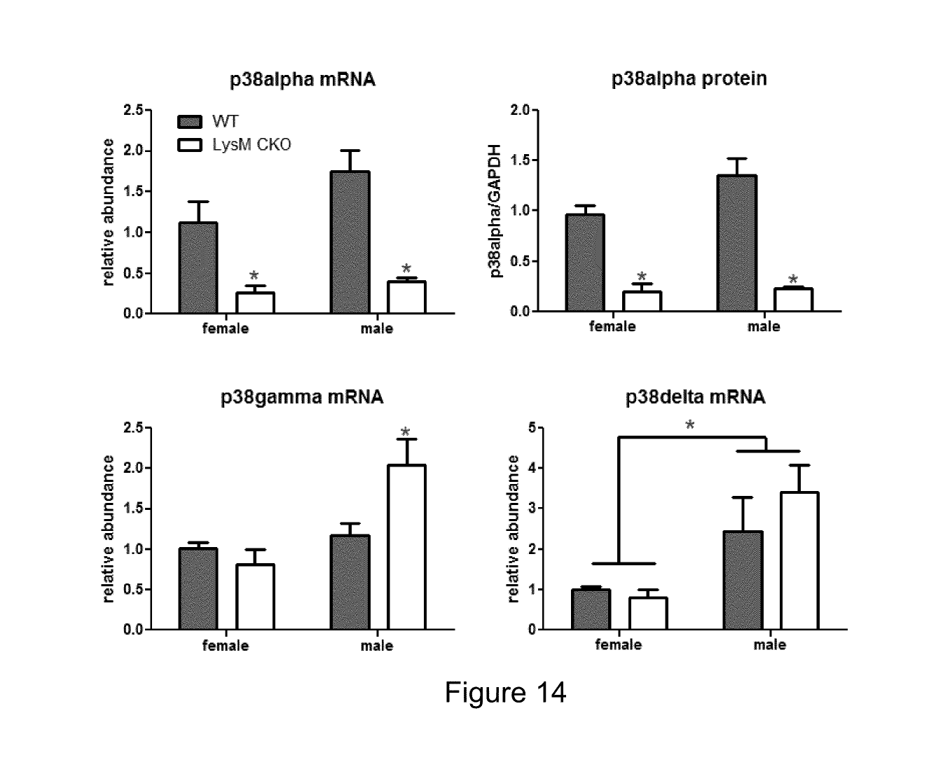

To examine potential mechanisms by which males do not experience the same decrease in EAE severity during p38 MAPK antagonism, the expression of various p38 isoforms were compared in male and female WT and p38CKO^{LysM} mice (FIG. 14). Both male and female knockout mouse demonstrated drastically reduced protein and mRNA levels of p38alpha, as expected. It was also observed that male knockout mice exhibited increased mRNA levels of alternate isoforms, p38gamma, and p38delta. Further, it appears as if WT male mice have greater levels of p38delta mRNA. While not wishing to be bound by any particular theory, this data suggests that male mice may bypass p38alpha signaling through increased expression of other p38 isoforms, thereby reducing or eliminating the effect of p38alpha antagonism.

Further experiments, relating to the transcriptomic profile of p38-controlled genes were performed in cells isolated from the CNS of wildtype and p38CKO^{LysM} male and female mice at the peak of diseases. Identification of genes that are p38-controlled and female-specific allows for ability to determine which genes are likely to responsible for the therapeutic response to p38 inhibition in females.

A recent study demonstrated that p38 MAPK activity in dendritic cells (DCs), and not T cells or myeloid cells, plays a key pro-inflammatory role in EAE by controlling Th17 cell generation (Huang et al., 2012, Nat Immunol, 13(2): 152-161). At first glance, these findings are partially in conflict with the data presented herein, since the authors found no effect of deleting p38 in myeloid cells. However, there are several important differences between this prior study and the data presented herein. The prior study: 1) did not report the sex of the animals used, 2) showed a specific effect on Th17 and not Th1 cells, and 3) used pertussis toxin (PTX) as an ancillary adjuvant in the EAE induction protocol. Because PTX can override many genetic checkpoints (Spach et al, 2009, J. Immunol., 182(12): 7776-7783), it was examined whether it can override protection provided by deletion of p38 in myeloid cells. It was found that this was indeed the case, since WT and p38CKO^{LysM} male and female mice exhibited no difference in EAE score when the 1×EAE induction protocol with PTX was used (data not shown). Thus, while not wishing to be bound by any particular theory, it appears that p38 can play different roles in different cell types, depending on the EAE model used.

Taken together, the present results suggest that p38 MAPK activity in T cells or myeloid cells can control severity of autoimmune disease of the CNS, however the extent of the involvement of either cell type is tightly controlled by genetic factors and gender. Importantly, p38 in the myeloid cell compartment appears to be responsible for the sexually dimorphic therapeutic response in B6 mice. These findings reveal important mechanisms underlying gender-specific differences in autoimmune disease, and suggest that the p38 MAPK pathway may present targets for female-specific DMTs for MS.

### Example 3

### Sex-Specific Control of CNS Autoimmunity by p38 MAPK Signaling in Myeloid Cells

Multiple sclerosis (MS) is a chronic inflammatory demyelinating disease of the central nervous system (CNS), characterized by a global increasing incidence driven by relapsing-remitting disease in females. p38 MAP kinase (MAPK) has been described as a key regulator of inflammatory responses in autoimmunity, but its role in the sexual dimorphism in MS or MS models remains unexplored.

The experiments presented herein used experimental autoimmune encephalomyelitis (EAE), the principal animal model of MS, combined with pharmacologic and genetic inhibition of p38 MAPK activity and transcriptomic analyses. As presented herein, pharmacologic inhibition of p38 MAPK selectively ameliorated EAE in female mice. Conditional deletion studies demonstrated that p38α signaling in macrophages/myeloid cells, but not T cells or dendritic cells, recapitulated this sexual dimorphism. Analysis of CNS inflammatory infiltrates showed that female, but not male mice lacking p38α in myeloid cells exhibited reduced immune cell activation compared with controls, while peripheral T cell priming was unaffected in both sexes. Transcriptomic analyses of myeloid cells revealed differences in p38α-controlled transcripts comprising female- and male-specific gene modules, with greater p38α dependence of pro-inflammatory gene expression in females. The data presented herein demonstrate a key role for p38α in myeloid cells in CNS autoimmunity and uncover important molecular mechanisms underlying sex differences in disease pathogenesis. Taken together, the results presented herein demonstrate that the p38 MAPK signaling pathway represents a novel target for much needed disease modifying therapies for MS.

The materials and methods employed in these experiments are now described.

Mice

Lysm-Cre mice (B6.129P2-Lyz2tm1(cre)Ifo/J) (Clausen B E, et al., Transgenic Res. 1999 August; 8(4):265-77), Cd11c-Cre mice (B6.Cg-Tg(Itgax-cre)1-1Reiz/J) (Caton M L, et al., J Exp Med. 2007 Jul. 9; 204(7):1653-64), p38α floxed mice (Mapk14^{tm1.2Otsu}) (Nishida K, et al., Mol Cell Biol. 2004 December; 24(24):10611-20) have been described previously and were obtained from Jackson Laboratories (USA) or RIKEN BioResource Center (Japan). Lck-Cre mice (B6.Cg-Tg(Lck-cre)1Cwi N9) (Lee P P, et al., Immunity. 2001 November; 15(5):763-74) were obtained from Taconic (USA). Wild type C57BL/6J mice were purchased from Jackson Laboratories (USA) and were rested for at least 2 weeks prior to any experimentation.

EAE Induction and Scoring

EAE was induced essentially as described previously (Noubade R, et al., J Clin Invest. 2007 November; 117(11):3507-18). Briefly, mice were injected s.c. with an emulsion containing 100 μg of MOG₃₅₋₅₅ peptide (MEVGWYRSPFSRVVHLYRNGK; SEQ ID NO: 1) (Anaspec, USA) and complete Freund's adjuvant (CFA) (Sigma-Aldrich, St. Louis, Mo.) supplemented with 200 μg of Mycobacterium tuberculosis H37Ra (Difco Laboratories, Detroit, Mich.) in the posterior right and left flanks. One week later all mice were similarly injected at two sites on the right and left flank anterior of the initial injection sites (2×MOG₃₅₋₅₅/CFA). Alternatively, mice were immunized with 200 μg of MOG₃₅₋₅₅ in CFA, followed by i.v. administration of 200 ng pertussis toxin (List Biological) (1×MOG₃₅₋₅₅/CFA/PTX). In some experiments (as indicated), mice received 5 mg/kg/day of SB203580 dihydrochloride (Tocris, Ellisville, Mo.) by i.p. injection in a total volume of 200 μl or an equal volume of carrier daily starting on the day of immunization. Mice were scored daily starting at day 10 post-injection as previously described (Noubade R, et al., J Clin Invest. 2007 November; 117(11):3507-18). Passive EAE was induced as follows. WT male and female mice were immunized with 2×MOG₃₅₋₅₅/CFA. On day 10, effector cells from LN and spleen were harvested and restimulated ex vivo with 10 μg/ml MOG₃₅₋₅₅ and 0.5 ng/ml IL-12 for 72 hours. 20×10⁶ sex-matched effector cells were transferred to female and male recipients.

Cytokine Quantification

For the detection of cytokines in the cell culture supernatants, ELISAs were performed as described previously (Noubade R, et al., J Clin Invest. 2007 November; 117(11):3507-18), using the primary capture mAbs: anti-IFNγ, anti-IL-17A, anti-TNFα, and anti-IL-6 and their corresponding biotinylated detection mAbs (BD Pharmingen, San Diego, Calif.). Other ELISA reagents included: HRP-conjugated avidin D (Vector Laboratories, Burlingame, Calif.), TMB microwell peroxidase substrate and stop solution (Kirkegaard and Perry Laboratories, Gaithersburg, Md.). rIFNγ, rIL-17A, rGM-CSF (Biolegend, USA), and rTNFα and rIL-6 (BD Pharmingen, San Diego, Calif.) were used as standards.

For analysis of antigen-specific cytokine production by lymphocytes from mice immunized with 2×MOG₃₅₋₅₅/CFA, spleen and draining lymph nodes (DLN) were harvested on day 10 post-immunization, single cell suspensions were prepared at 1×10⁶ cells/ml in RPMI medium with 5% FBS, and stimulated with 50 μg/ml of MOG₃₅₋₅₅. Cell culture supernatants were collected at 72 hours and cytokine levels were measured by ELISA as described above.

Isolation and Stimulation of Thioglycolate-Elicited Macrophages

Mice were immunized using the 2×MOG₃₅₋₅₅/CFA protocol. On day 6 post-immunization, mice were injected with 1 ml of a 4% solution of thioglycolate broth (Sigma-Adrich, USA) i.p. 96 hours later mice were sacrificed and the peritoneal cavity was flushed with 15 ml of cold PBS. Cells were washed and cultured overnight in RPMI+5% FBS, then washed to remove non-adherent cells. The remaining adherent cells were stimulated with purified LPS (Sigma) or heat-killed Mycobacterium tuberculosis H37Ra (Difco, USA).

Flow Cytometry

For intracellular cytokine staining ex vivo, mice were immunized with 2×MOG₃₅₋₅₅/CFA, spleen and DLN were harvested on day 10 post-immunization, and cells were stimulated with 5 ng/ml of PMA, 250 ng/ml of ionomycin (Sigma-Aldrich) and Golgi Plug reagent (BD Biosciences) for 4 hours. Cells were then stained with the LIVE/DEAD fixable stain (Invitrogen) and then surface stained for the following markers: CD11b, CD4, CD8, TCRγδ, and TCRβ. Cells were then fixed with 1% paraformaldehyde (Sigma-Aldrich), permeabilized with buffer containing 0.2% saponin and stained with anti-IL-17A, anti-IFNγ, and anti-GM-CSF (Biolegend).

For surface marker analysis, unstimulated isolated cells were stained directly ex vivo with the LIVE/DEAD fixable stain (Invitrogen) and then surface labeled for different combinations of following markers: CD11b, CD11c, MHCII, CD80, CD86, Ly6C, Ly6G, MHCII, CD4, CD8, TCRγδ, and TCRβ (Biolegend, USA) and fixed with 1% paraformaldehyde. All antibodies used for flow cytometry were directly conjugated to fluorophores.

Cells were analyzed using an LSR II cytometer (BD Biosciences). Compensation was calculated using appropriate single color controls. Data were analyzed using FlowJo software (Tree Star Inc, Ashland, Oreg.).

CNS-Infiltrating Mononuclear Cell Isolation

Animals were perfused with saline and brains and spinal cords were removed. A single cell suspension was obtained and passed through a 70 μm strainer. Mononuclear cells were obtained by Percoll gradient (37%/70%) centrifugation and collected from the interphase. For mRNA analysis, cells were lysed and total RNA was isolated using the RNEasy kit (Qiagen). For intracellular cytokine analysis, cells were washed and stimulated with 50 μg/ml of MOG₃₅₋₅₅ for 4 hours in the presence of Golgi Plug reagent (BD Bioscience). Cells were labeled with LIVE/DEAD UV-Blue dye (Invitrogen) followed by surface staining (anti-CD45 from Invitrogen and anti-CD11b, CD11c, Ly6C, Ly6G, MHCII, CD4, CD8, TCRγδ, and TCRβ from Biolegend). Afterwards, cells were fixed, permeabilized and stained for intracellular IL-17A, IFNγ, and GM-CSF (Biolegend) as described above. For surface marker analysis, unstimulated isolated cells were stained directly ex vivo for the following markers: CD45, CD11b, CD11c, MHCII, CD80, CD86, Ly6C, Ly6G, MHCII, CD4, CD8, TCRγδ, and TCRβ.

Cell Lysates and Immunoblot Analysis

Whole-cell lysates were prepared by lysing adherent macrophages directly in Triton lysis buffer, separated by SDS-PAGE, and transferred to PVDF membranes as described previously (Noubade R, et al., J Clin Invest. 2007 November; 117(11):3507-18.). Primary antibodies used for western blot analysis included anti-phospho-p38, anti-p38α, and anti-GAPDH (Cell Signaling Technologies, Danvers, Mass.). Anti-mouse and anti-rabbit secondary antibodies were conjugated to DyLight680 and DyLight800, respectively (Jackson ImmunoResearch Laboratories, West Grove, Pa.). Membranes were imaged using fluorescent detection on the Odyssey CLx instrument (Li-Cor Biosciences, USA), and images were processed using the Image Studio program (Li-Cor Biosciences, USA).

RNA Isolation and Quantitative Real-Time PCR (qRT-PCR)

RNA was extracted using the RNEasy kit (Invitrogen) according to manufacturer's instructions. cDNA was reverse transcribed using the Taqman Gold RT-PCR kit using the oligo-dT method (Applied Biosciences, USA). qRT-PCR was performed using the DyNAmo Colorflash SYBR green qPCR kit (Thermofisher) and the following primer sets:

| | |
|---|---|
| I110, gaagctgaagaccctcagga | (SEQ ID NO: 4) |
| and | |
| ttttcacaggggagaaatcg; | (SEQ ID NO: 5) |
| Tnfa, GAACTGGCAGAAGAGGCACT | (SEQ ID NO: 6) |
| and | |
| AGGGTCTGGGCCATAGAACT; | (SEQ ID NO: 7) |
| I16, CCGGAGAGGAGACTTCACAG | (SEQ ID NO: 8) |
| and | |
| GAGCATTGGAAATTGGGGTA; | (SEQ ID NO: 9) |
| I11b, AGGCCACAGGTATTTTGTCG | (SEQ ID NO: 10) |
| and | |
| GCCCATCCTCTGTGACTCAT; | (SEQ ID NO: 11) |
| B2m, CATGGCTCGCTC GGTGACC | (SEQ ID NO: 12) |
| and | |
| AATGTGAGGCGGGTGGAACTG. | (SEQ ID NO: 13) |

B2m was used as a reference gene and relative mRNA levels were calculated using the comparative delta-delta C_{T} method, normalizing first by the expression of the reference gene, then normalizing to the mean WT female expression level for each gene of interest.

Microarray Sample Preparation and Hybridization

RNA was isolated using the RNEasy kit (Qiagen) according to manufacturer's instructions. Microarray was performed on 3 biological replicates for each condition (e.g. female KO). To create each replicate, equal amounts of RNA from 2-3 different mice were pooled.

An RNA input of 25 ng was used to generate cDNA through the First Strand and Second Strand synthesis reactions of the Ovation® Pico WTA System V2 from NuGEN. The cDNA samples were then purified using an Agencourt® RNAClean® XP magnetic bead protocol. Following purification, samples were amplified using SPIA reagents from the Ovation® Pico WTA System V2 from NuGEN. A final cDNA purification is performed using an Agencourt® RNAClean® XP magnetic bead protocol. Sample concentrations were determined using a 33 ug/mL/A260 constant on a Nanodrop 1000 Spectrophotometer. Approximately 4 ug of cDNA generated using the Ovation® Pico WTA System V2 was fragmented and labeled using the Encore® Biotin Module from NuGEN. Efficiency of the biotin labeling reaction was verified using NeutrAvidin (10 mg/mL) with a gel-shift assay. Samples were injected into arrays and placed in the Affymetrix Genechip® Hybridization Oven 640 at 45° C. and 60 RPM for 16-18 hours overnight. Arrays were stained using the Affymetrix Genechip® Fluidics Station 450 and scanned with the Affymetrix Genechip® Scanner 3000. Mouse Gene 2.0 ST arrays were used (Affymetrix).

Microarray Analysis—Calculation of Probe Set Statistics

Raw GeneChip data (one DAT file for each chip) includes a collection of images, one for each probe and chip. Each image was summarized by Affymetrix GCOS software using one probe intensity (in CEL files, one per chip). Information from multiple probes can be combined to obtain a single measure of expression for each probe set and sample. Probe-level intensities were calculated using the Robust Multichip Average (RMA) algorithm, including background-correction, normalization (quantile), and summarization (median polish), for each probe set and sample, as is implemented in Partek Genomic Suites®, version 6.6 (Copyright © 2009, Partek Inc., St. Louis, Mo., USA). Sample quality was assessed based on the 3′:5′ ratio (3′ arrays only), relative log expression (RLE), and normalized unscaled standard error (NUSE).

Microarray Analysis—Identification of Differential Expression and Alternative Splicing

Univariate linear modeling of sample groups is performed using ANOVA as implemented in Partek Genomic Suites. The magnitude of the response (fold change calculated using the least square mean) and the p-value associated with each probe set and binary comparison are calculated, as well a “step-up,” adjusted p-value for the purpose of controlling the false discovery rate (FDR) (Benjamini Y, et al., Journal of the Royal Statistical Society Series B (Methodological). 1995; 57(1):289-300). For identification of differentially expressed genes between WT and KO in females and males, a binary filter of |FC|>1.5 and p<0.05 was used. ANOVA was also performed to detect alternative splicing, using exon-specific probe sets (at least one probe per exon). A filter of FDR<0.05 was used as a cut off for alternative splicing analysis.

Bioinformatic Identification of Biological Processes Associated with Differentially Expressed Genes

Data were analyzed through the use of Ingenuity Pathways Analysis (IPA; Ingenuity® Systems). Lists of differentially expressed genes in females and males were uploaded and analyzed using IPA Core Analysis. The Upstream Analysis function was used to identify predicted upstream regulators. The overlap p value generated by Upstream Analysis indicates the significance of the number of genes in the data set regulated by a given upstream regulator. The Z-score indicates the predicted direction of change for a given upstream regulator, with the sign indicating repression (negative) or upregulation (positive). Estrogen (beta-estradiol) and testosterone (dihydrotestosterone) were both in the top ten upstream regulators with the lowest Z-score (indicative of inactivation in p38α-deficient cells) for female and male data sets, respectively.

Statistical Analyses

All statistical analyses were performed using GraphPad Prism 6 software (GraphPad Software Inc, San Diego, Calif.). The significance of differences in cytokine production and flow cytometry data were determined using 2-way ANOVA. The significance of differences observed in clinical course of EAE was determined by 2-way ANOVA and post-hoc analysis using Fisher's LSD test for individual time points (significance indicated by “*” above each time point). Non-linear regression analyses of the mean daily clinical disease scores indicated that the disease course amongst all strain and treatment combinations was best fit by a variable slope dose response curve, which together with daily mean score was used to represent the change in clinical disease over time. EAE data from replicate experiments were analyzed by heterogeneity testing. No significant experiment-to-experiment variation was observed, and therefore the data were pooled accordingly.

The results of the experiments are now described.

### Pharmacologic Inhibition of p38 MAPK Signaling Ameliorates EAE in a Sex-Specific Manner

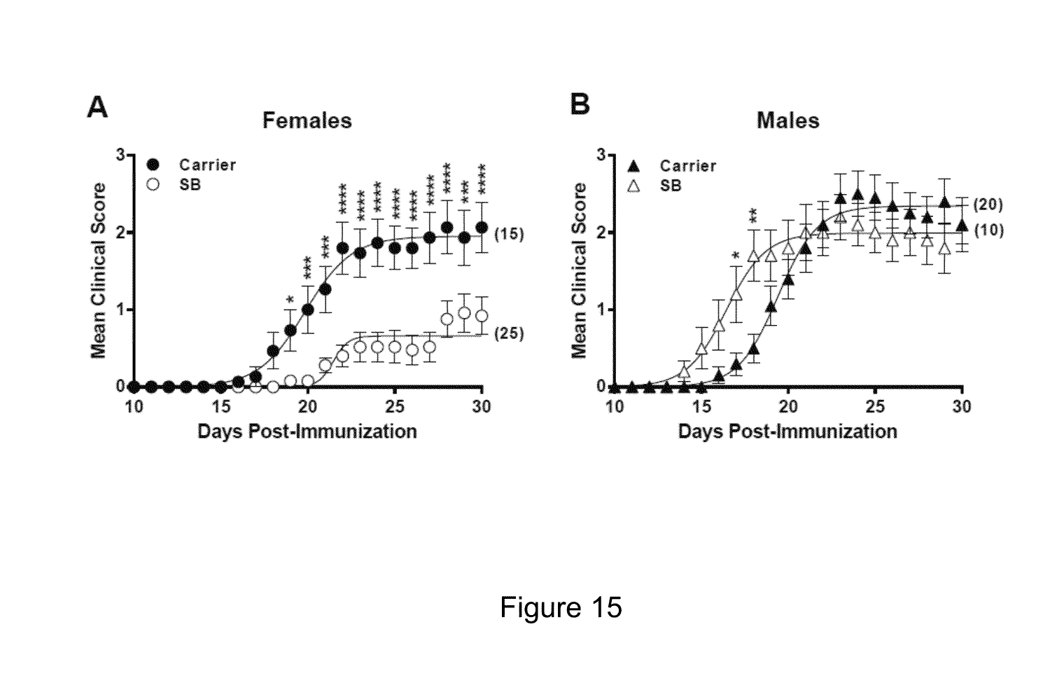

It has been previously shown that pharmacologic inhibition of p38 MAPK prevented EAE in female C57BL/6J (B6) mice (Noubade R, et al., Blood. 2011 Sep. 22; 118(12):3290-300). Since EAE, like MS, can often exhibit sexual dimorphisms (Spence R D, et al., Front Neuroendocrinol. 2012 January; 33(1):105-15), it was examined whether male mice showed a similar therapeutic response. EAE was induced in female and male B6 mice using the 2×MOG₃₅₋₅₅/CFA protocol, followed by daily treatment with SB203580, a small molecule inhibitor of p38α and β. Surprisingly, while disease was robustly ameliorated in female mice as previously described (FIG. 15A), SB203580 treatment showed no therapeutic efficacy in male mice; in fact, EAE onset was accelerated (FIG. 15B). Thus, the therapeutic response to p38 MAPK inhibition is sexually dimorphic.

p38α Signaling in Myeloid Cells Underlies the Sexually Dimorphic Therapeutic Efficacy of p38 MAPK Inhibition in EAE

To delineate the contribution of p38 MAPK signaling in different cell types to the sexually dimorphic response to SB203580 treatment, as well as to rule out any sex-specific pharmacokinetic differences, cell type-specific genetic ablation of p38α, the predominant isoform expressed in immune cells, was used. Autoreactive Th1 and Th17 cells are thought to initiate disease in both MS and EAE (Segal B M, Semin Immunopathol. 2010 March; 32(1):71-7), and p38 MAPK signaling in Th cells plays an important role in the generation and function of both Th1 and Th17 cells(Lu L, et al., J Immunol. 2010 Apr. 15; 184(8):4295-306; Noubade R, et al., Blood. 2011 Sep. 22; 118(12):3290-300; Namiki K, et al., J Biol Chem. 2012 Jul. 13; 287(29):24228-38; and Rincon M, et al., EMBO J. 1998 May 15; 17(10):2817-29). Moreover, conventional dendritic cells (DCs) are important in the activation of pathogenic Th cells not only in the lymphoid organs, but also in the CNS during disease progression (Chastain E M, et al., Biochim Biophys Acta. 2011 February; 1812(2):265-74), and p38α signaling in DCs has been recently shown to promote the generation of encephalitogenic Th17 cells (Huang G, et al., Nat Immunol. 2012 February; 13(2):152-61). Lastly, myeloid cells such as macrophages, microglia, and neutrophils are important mediators of tissue destruction and inflammation in the CNS during EAE and MS (Izikson L, et al., Clin Immunol. 2002 May; 103(2):125-310), and p38α is thought to control the release of many pro-inflammatory mediators from these cells (Rincon M, et al., Immunol Rev. 2009 March; 228(1):212-24). Therefore, it was examined whether the sex-specific therapeutic efficacy of SB203580 (FIG. 15) is mediated by inhibition of p38 MAPK signaling in one or more of these cell types. For these studies, B6 mice expressing a floxed allele of p38α (p38^{fl/fl} (Nishida K, et al., Mol Cell Biol. 2004 December; 24(24):10611-20) were crossed to mice expressing Cre recombinase under the control of the Lck, Cd11c, or Lysm/Lyz2 promoters (Clausen B E, et al., Transgenic Res. 1999 August; 8(4):265-77; Caton M L, et al., J Exp Med. 2007 Jul. 9; 204(7):1653-64; and Lee P P, et al., Immunity. 2001 November; 15(5):763-74). This selectively ablates p38α in T cells (p38CKO^{Lck}), conventional DCs (p38CKO^{Cd11c}), or myeloid cells (p38CKO^{Lysm}), respectively.

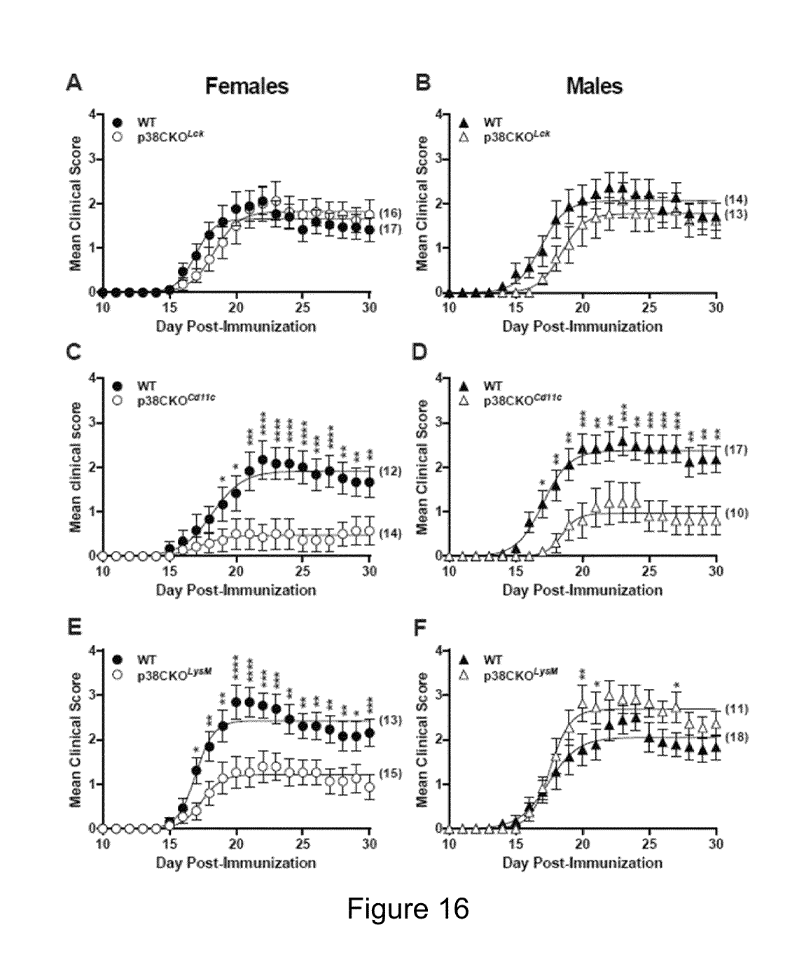

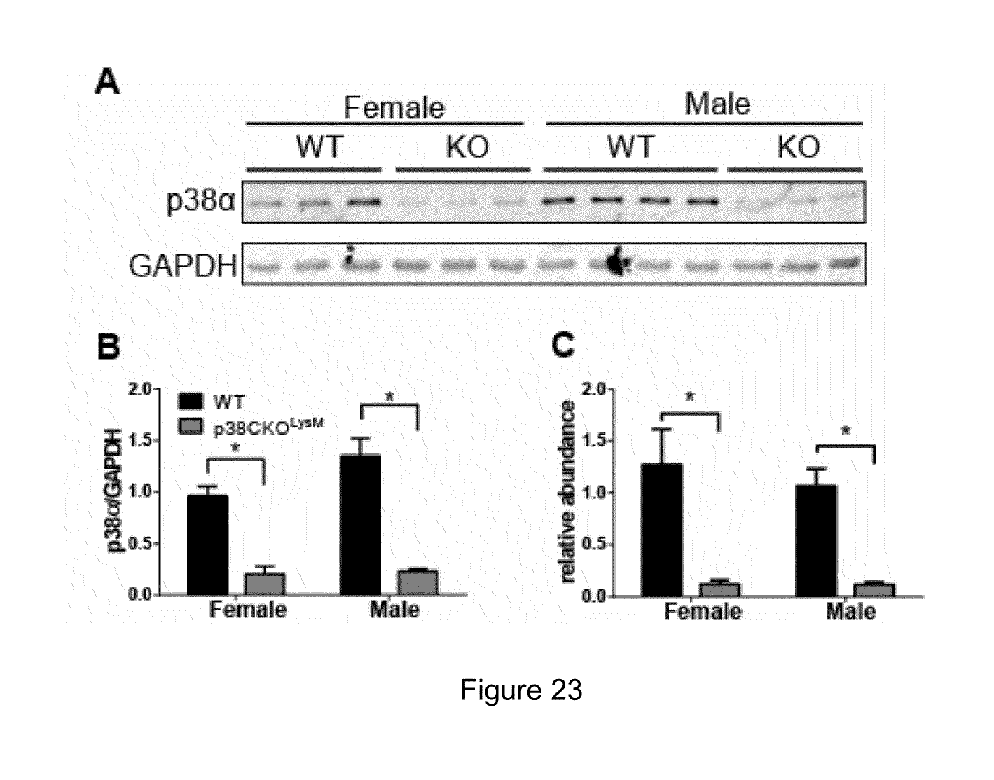

EAE was induced in the resulting mice using the 2×MOG₃₅₋₅₅/CFA protocol. Surprisingly, p38CKO^{Lck} mice exhibited a disease course similar to littermate p38α^{fl/fl} Cre-negative controls (these are designated as WT throughout), suggesting that p38α in T cells is not essential for EAE in B6 mice (FIG. 16A and FIG. 16B). Deletion of p38α in DCs ameliorated disease, as recently reported (Huang G, et al., Nat Immunol. 2012 February; 13(2):152-61), but in a non sex-specific manner (FIG. 16C and FIG. 16D). However, deletion of p38α in myeloid cells resulted in diminished disease in females, but not males (FIG. 16E and FIG. 16F). EAE in males was in fact augmented by deletion of p38α in myeloid cells (FIG. 16F), as seen with SB203580 treatment (FIG. 15B). Expression of any of the above Cre alleles in p38α^{wt/fl} or p38α^{wt/wt} mice did not affect EAE, thereby excluding any non-specific effects of Cre expression. Furthermore, p38α was deleted with equal efficiency in myeloid cells from female and male p38CKO^{Lysm} mice (FIG. 23). Taken together, these results demonstrate that inhibition of p38α in myeloid cells underlies the sexual dimorphism observed with pharmacologic inhibition of p38 MAPK (FIG. 15A).

p38α Signaling in Myeloid Cells Promotes CNS Inflammation

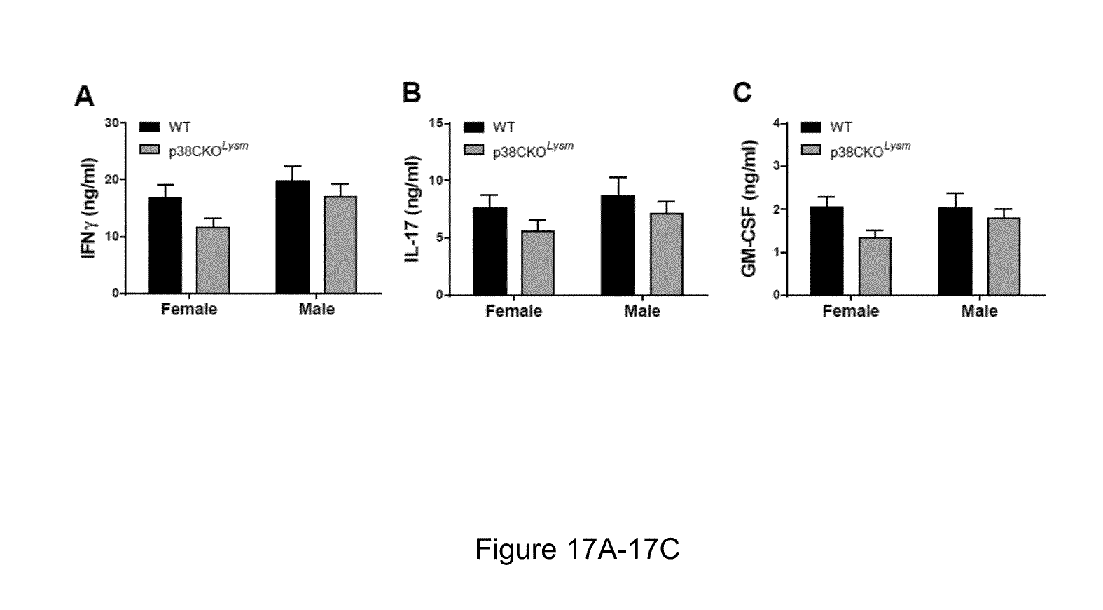

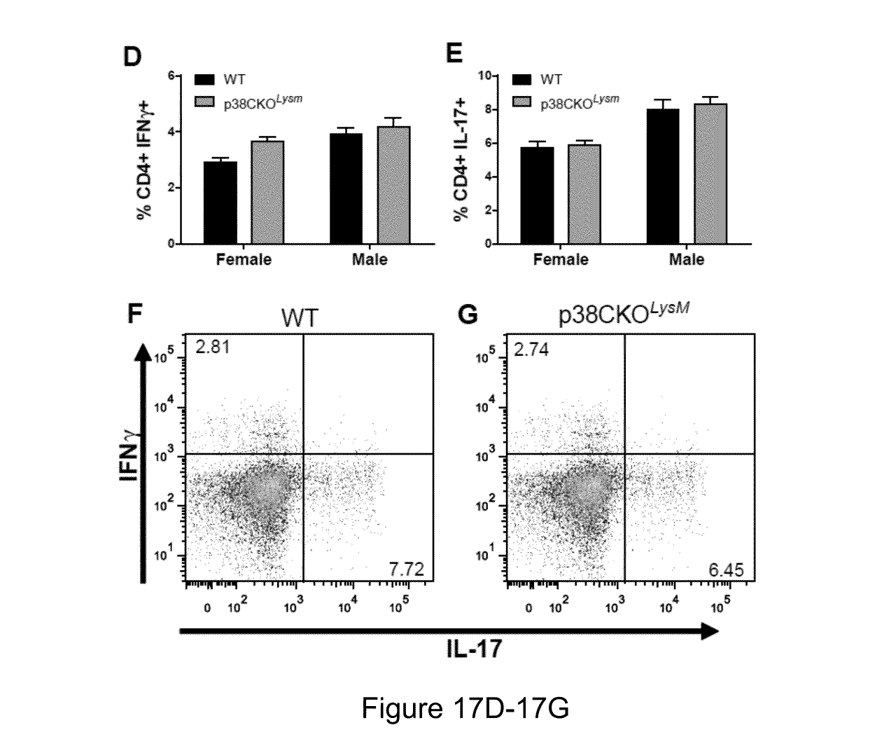

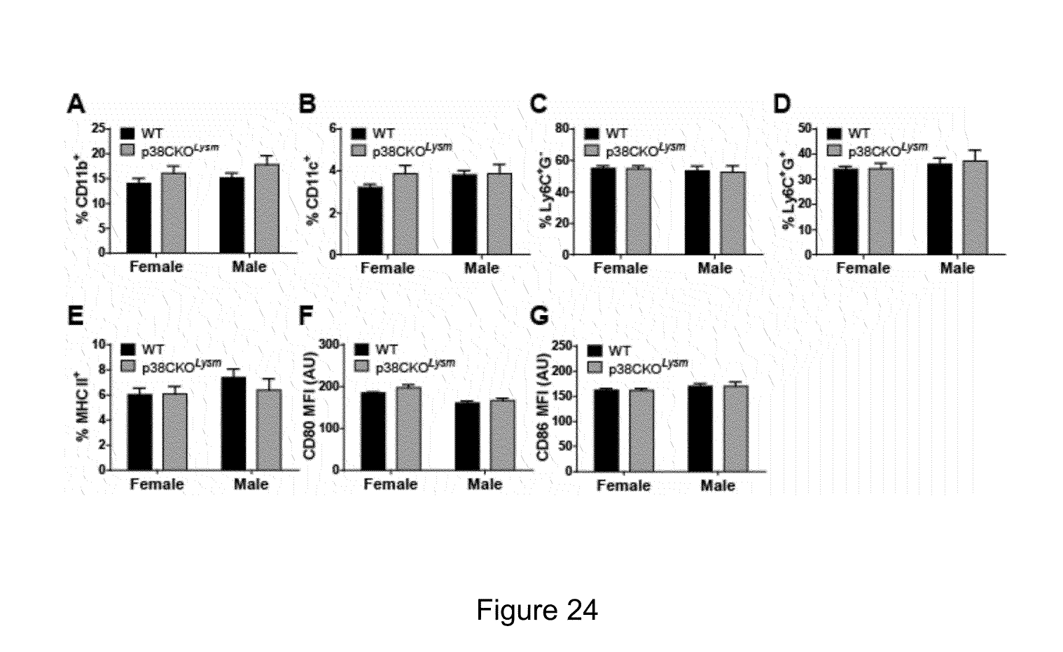

It was examined whether deletion of p38α in myeloid cells affected their proinflammatory functions selectively in females. Myeloid cells such as macrophages are potent antigen-presenting cells (APCs) that can influence T cell priming and effector responses by presenting antigen and regulating the cytokine milieu (Chastain E M, et al., Biochim Biophys Acta. 2011 February; 1812(2):265-740). Thus, it was first determined whether deletion of p38α in myeloid cells affected the priming of myelin-specific Th1 and Th17 cells in secondary lymphoid organs. p38CKO^{Lysm} mice and WT control littermates were immunized with 2×MOG₃₅₋₅₅/CFA, and 10 days later Th1 and Th17 responses in lymph nodes (LN) and spleen were assessed using ex vivo cytokine staining or by measuring MOG₃₅₋₅₅-stimulated cytokine production by ELISA. No significant effect of p38α deletion on the production of IFNγ, IL-17, or GM-CSF was found in female or male mice (FIG. 17). Moreover, normal numbers and percentages of myeloid cells were found in lymphoid tissues of p38CKO^{Lysm} mice, and the expression of MHC Class II and co-stimulatory molecules CD80 and CD86 on these cells was not affected (FIG. 24). Similar results were obtained in thioglycolate-elicited peritoneal macrophages. Taken together, these results demonstrate that p38α in myeloid cells is dispensable for normal myeloid cell homeostasis and for efficient priming of peripheral Th1 and Th17 responses.

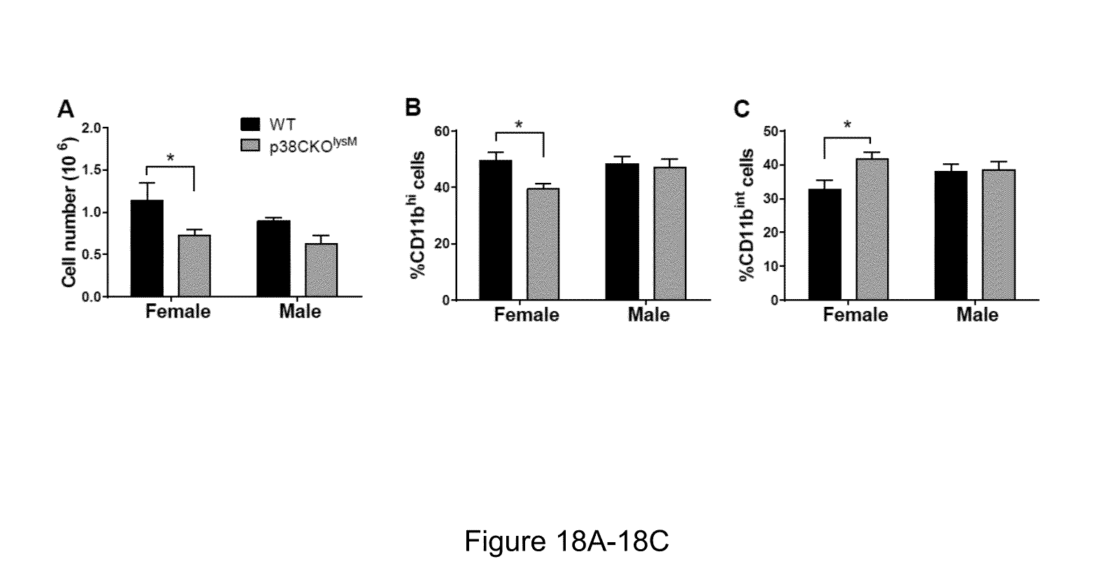

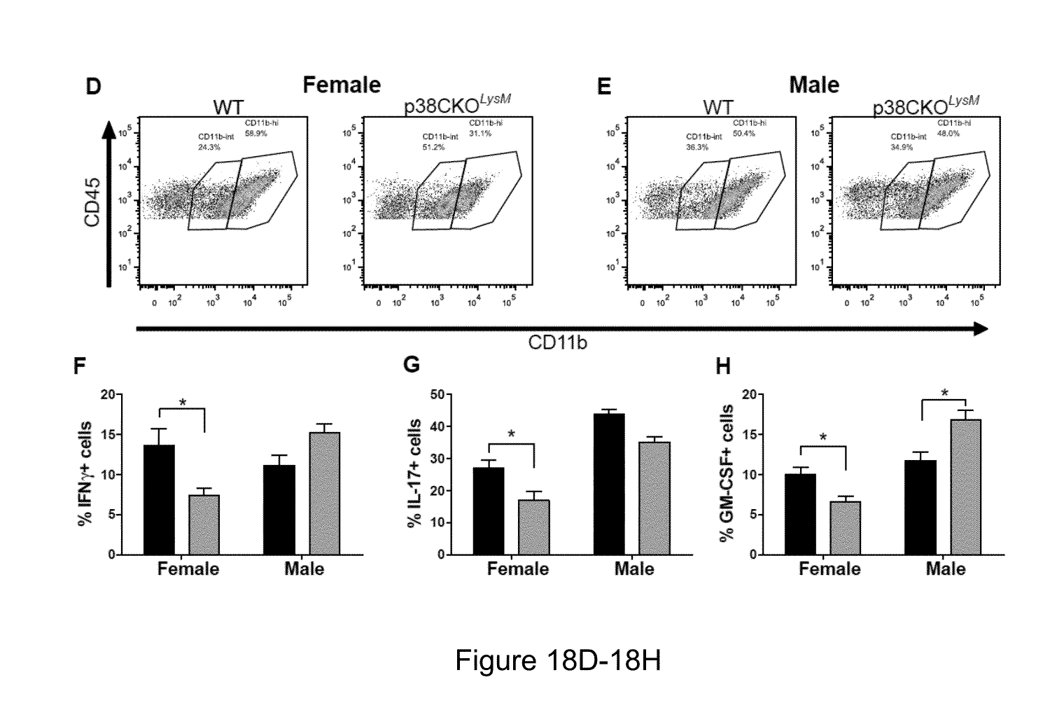

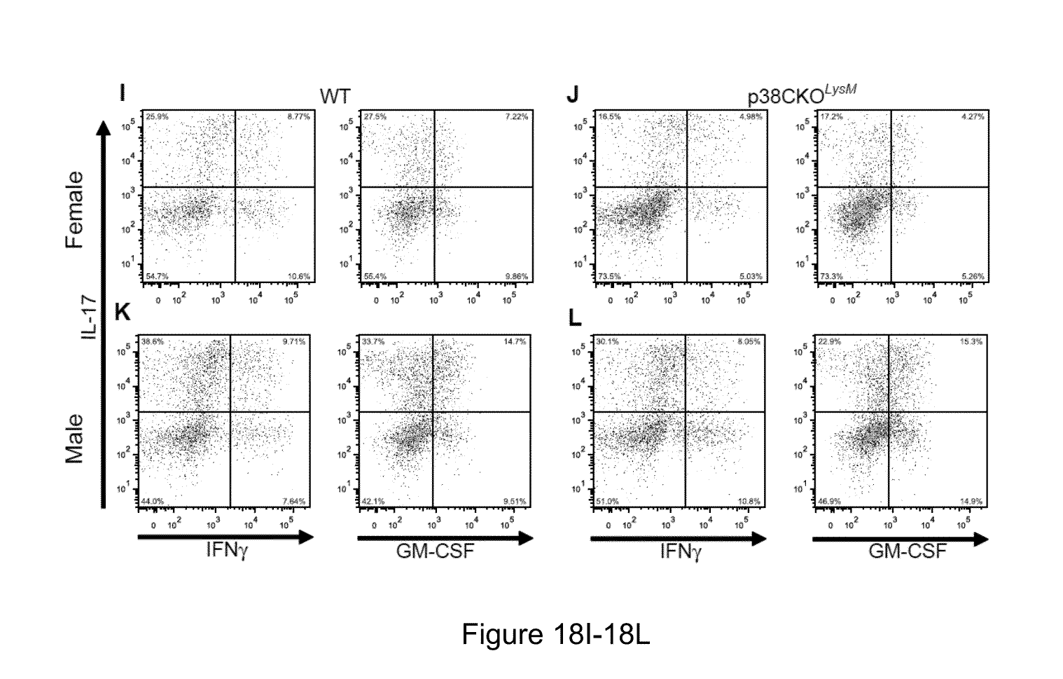

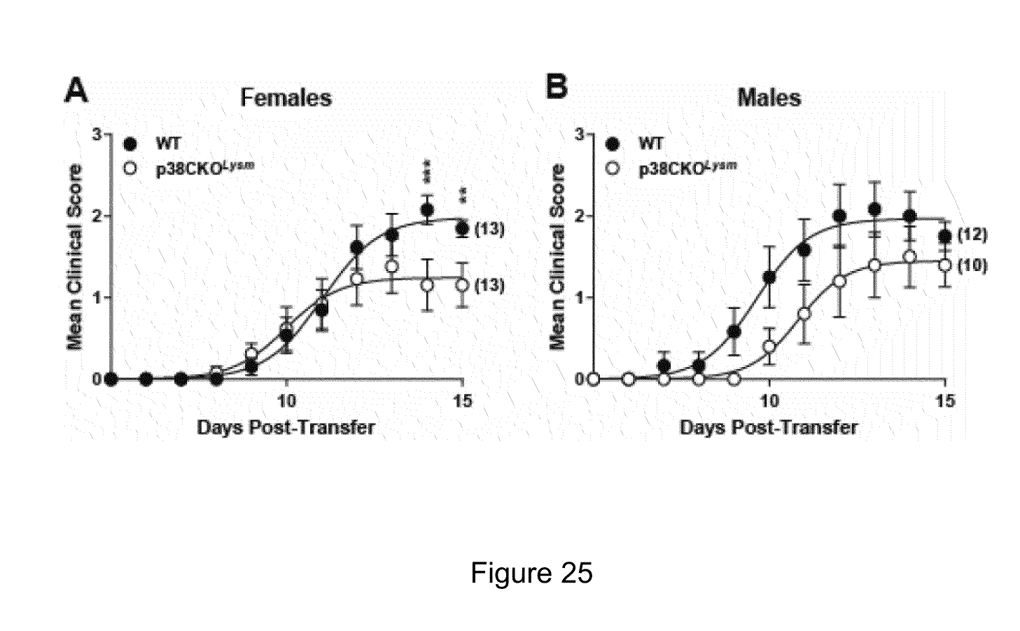

It was next assessed whether deletion of p38α in myeloid cells affected the inflammatory response in the CNS, since peripheral immune responses may not fully represent what occurs in the target organ. p38CKO^{Lysm} mice and WT control littermates were immunized with 2×MOG₃₅₋₅₅/CFA, and infiltrating mononuclear cells were isolated from the CNS at the peak clinical disease (day 19). Total mononuclear cell numbers were significantly reduced in p38CKO^{Lysm} female mice compared to WT females (FIG. 18A), suggesting reduced infiltration of immune cells into the CNS. CD11b expression on macrophages and microglia is upregulated by activation of these cells during neuroinflammation (Ponomarev E D, et al., J Neurosci Res. 2005 Aug. 1; 81(3):374-89). There was a reduction in the percentage of activated CD11b^{hi} myeloid cells, with corresponding increase in the CD11b^{int} population in female p38CKO^{Lysm} mice (FIG. 18B-FIG. 18E). Furthermore, production of IFNγ, IL-17, and GM-CSF by CD4 T cells was reduced in p38CKO^{Lysm} female mice compared to WT females (FIG. 18F-FIG. 18L). None of these changes were seen in male p38CKO^{Lysm} mice compared to WT males; in fact, GM-CSF production was significantly increased (FIG. 18H). Taken together, these results indicate that in females p38α in myeloid cells promotes CNS inflammation and indirectly promotes CNS T cell responses, whereas in males it may play an opposing role. To further verify that myeloid cell-specific deletion of p38α impacted the effector phase of EAE, passive EAE was induced in WT or p38CKO^{Lysm} mice by adoptive transfer of effector T cells harvested from WT sex-matched donors. EAE severity in p38CKO^{Lysm} female mice was significantly reduced compared to WT females (FIG. 25A), while in males no significant difference was observed (FIG. 25B).

p38α Signaling in Myeloid Cells is not Required for TLR-Induced TNFα and IL-6 Production

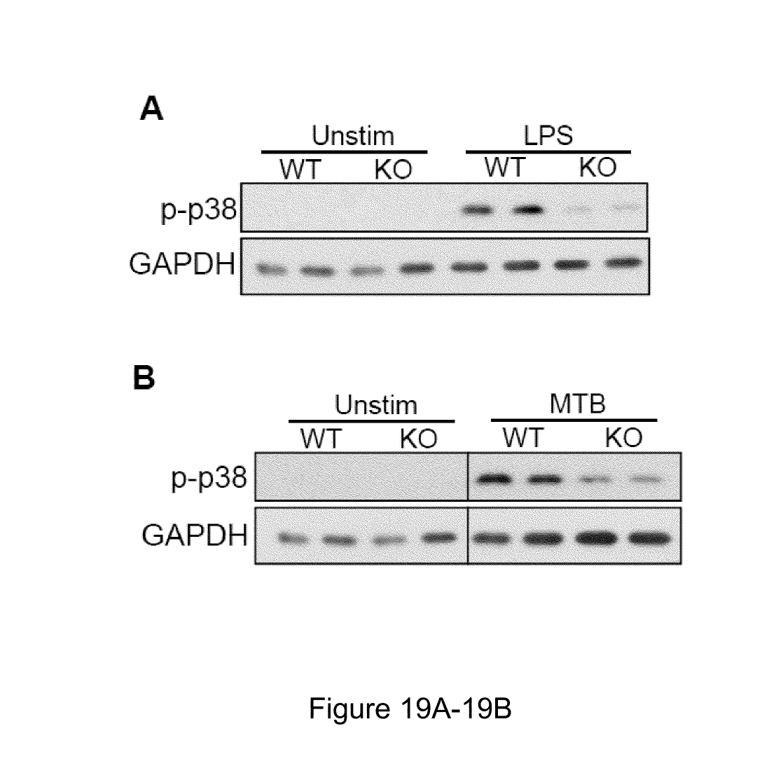

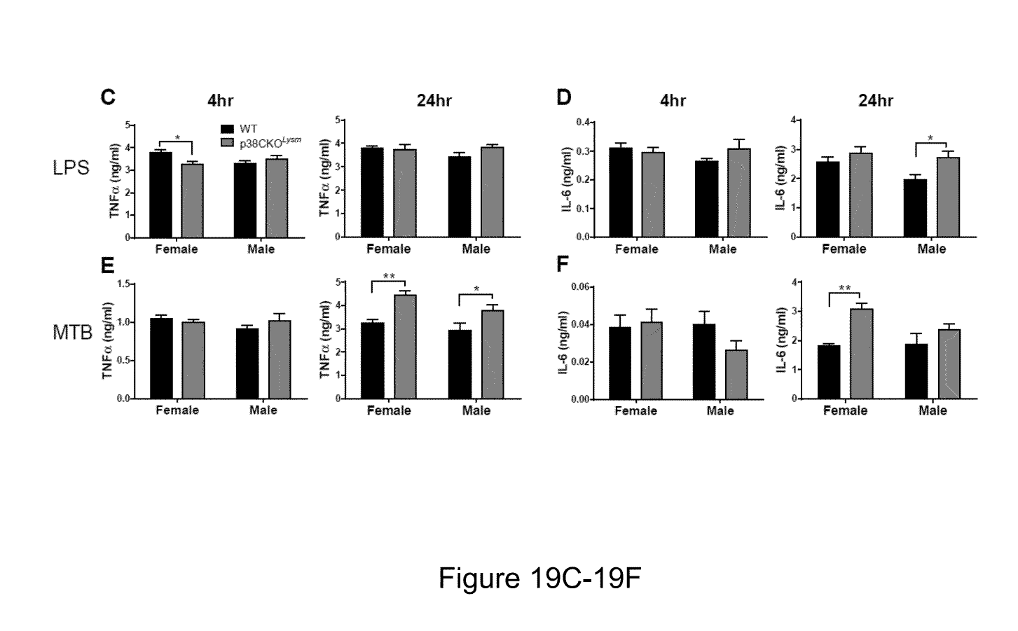

It was next examined whether p38α controls a subset of pro-inflammatory mediators in female myeloid cells. p38 MAPK has long been known to control the production of proinflammatory cytokines by macrophages in response to TLR stimulation. Therefore the effect of p38α ablation was tested on macrophage responses to TLR agonists. Thioglycolate-elicited macrophages were isolated from WT and p38CKO^{Lysm} mice immunized with 2×MOG₃₅₋₅₅/CFA in order to more closely mimic the in vivo environment to which myeloid cells are exposed during EAE. Macrophages were stimulated ex vivo by LPS, a TLR4 agonist, or heat-killed Mycobacterium tuberculosis H37Ra (MTB), the primary adjuvant used to induce EAE which contains several TLR ligands (Marta M, et al., Autoimmun Rev. 2009 May; 8(6):506-9)). These stimuli resulted in increased phosphorylation of p38 MAPK, which was strongly reduced in p38CKO^{Lysm} mice (FIG. 19A and FIG. 19B). However, the production of TNFα and IL-6, two TLR-induced cytokines that have been previously shown to be controlled by p38 MAPK (Rincon M, et al., Immunol Rev. 2009 March; 228(1):212-24), was not reduced in macrophages from female or male p38CKO^{Lysm} mice (FIG. 19C-FIG. 19F). Production of these two cytokines was in fact modestly enhanced in p38CKO^{Lysm} macrophages relative to WT at several of the time points assayed, but this was not sex-specific. Similarly, no sex-specific differences in the production of these cytokines were observed using bone marrow-derived macrophages. These results demonstrate that production of cytokines classically associated with p38 MAPK signaling is not reduced in p38CKO^{Lysm} macrophages, and hence does not explain the sex-specific effects of p38α deletion on EAE susceptibility.

p38α Signaling in Myeloid Cells Controls Unique Sex-Specific Gene Expression Modules

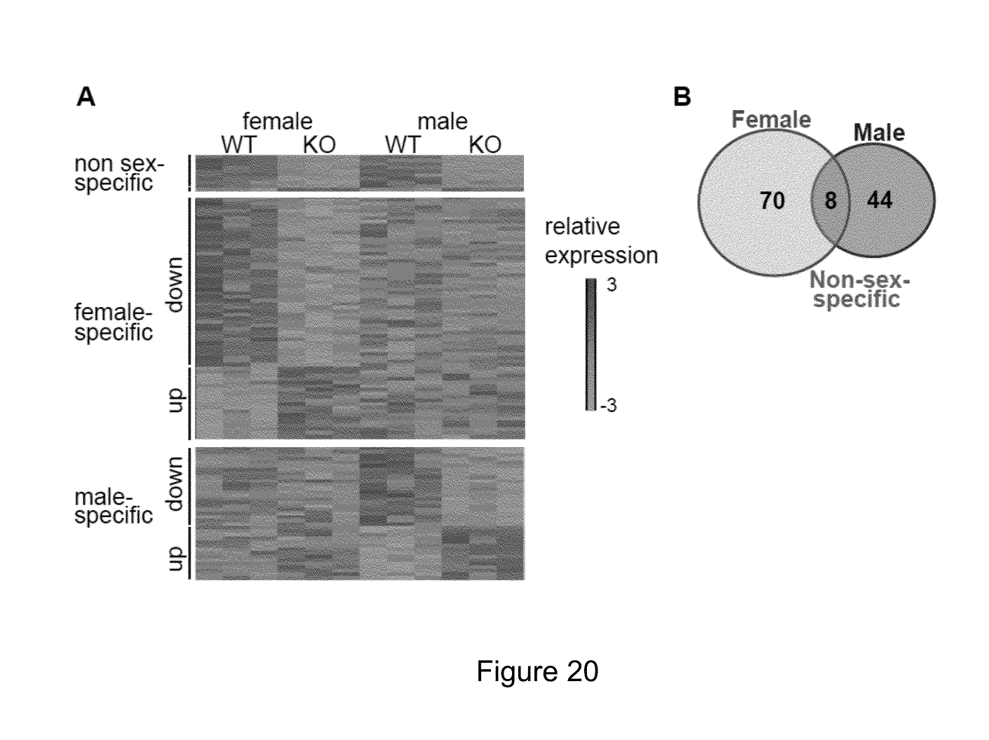

Since cytokines classically associated with p38 MAPK signaling in macrophages were not altered by p38α deletion in a sex-specific manner, a genome-wide transcriptomic approach was utilized to identify sex-specific p38α-regulated transcripts in macrophages. Thioglycolate-elicited macrophages were isolated from WT and p38CKO^{Lysm} female and male mice immunized using the 2×MOG₃₅₋₅₅/CFA protocol, and restimulated ex vivo with MTB for 4 hours, at which point RNA was extracted and subjected to microarray analysis. Differential expression analysis revealed three unique modules of p38α-dependent genes in macrophages: non-sex-specific (i.e. controlled by p38α in both sexes), female-specific, and male-specific (Table 1-Table 3, FIG. 20A and FIG. 20B). The number p38α-dependent transcripts was higher in females than in males, with limited overlap (70 vs. 44 genes; 8 genes in common), suggesting a greater dependence on p38α in females.

p38α Differentially Regulates Pro- and Anti-Inflammatory Genes in Females and Males

MS and EAE are polygenic diseases, where small effects of multiple loci contribute to overall disease susceptibility (Oksenberg J R, et al., Ann Neurol. 2011 December; 70(6):A5-7; Gourraud P A, et al., Immunol Rev. 2012 July; 248(1):87-103). By analogy, while not wishing to be bound by any particular theory, it is hypothesized that the effect of p38α deletion in EAE is mediated by the combined effects of multiple genes within the p38α-dependent modules, rather than a single key p38α-dependent gene. Within the non-sex-specific module, several genes of interest were downregulated in the absence of p38α (Table 1). Serpinb2 (plasminogen activator inhibitor 2, PAI-2) and Mmp13 (matrix metalloproteinase 13, MMP-13) have both been previously shown to be controlled by p38α in macrophages, where PAI-2 can inhibit apoptotic responses and IL-1β production (Park J M, et al., Immunity. 2005 September; 23(3):319-29; Kim C, et al., Nat Immunol. 2008 September; 9(9):1019-27; and Greten F R, et al., Cell. 2007 Sep. 7; 130(5):918-31). Although the role of MMP-13 in EAE/MS is so far unexplored, MMPs are well-known to be involved in the pathogenesis of these diseases (Rosenberg G A, Glia. 2002 September; 39(3):279-91). Il1f9 encodes IL-36γ, a novel cytokine with a potential role in psoriasis (Carrier Y, et al., J Invest Dermatol. 2011 December; 131(12):2428-37; Tortola L, et al., J Clin Invest. 2012 Nov. 1; 122(11):3965-76; and Vigne S, et al., Blood. 2011 Nov. 24; 118(22):5813-23), a Th17-driven autoimmune disease. Mirlet7e encodes a microRNA that was recently shown to promote EAE pathogenesis (Guan H, et al., Eur J Immunol. 2013 January; 43(1):104-14). In contrast, Ccr5 (chemokine (C-C motif) receptor 5), which was also downregulated in the absence of p38α, is not required for EAE (Rottman J B, et al., Eur J Immunol. 2000 August; 30(8):2372-7). It is important to note that while these genes did not exhibit sex-specific p38α-dependence, their altered expression may nevertheless contribute to the observed sex-specific phenotypes in EAE by interacting with genes within the female- or male-specific p38α-controlled modules.

In females, deletion of p38α downregulated several genes that are known to promote EAE or MS pathogenesis (Table 2). The product of Maoa, monoamine oxidase A, has been successfully targeted to treat EAE (Musgrave T, et al., Brain Behav Immun. 2011 November; 25(8):1677-88). Oas1g (2′-5′ oligoadenylate synthetase 1G) is an ortholog of human OAS1, which was recently found to be associated with MS susceptibility and severity (O'Brien M, et al., Neurology. 2010 Aug. 3; 75(5):411-8; Fedetz M, et al., Tissue Antigens. 2006 November; 68(5):446-9; and Cagliani R, et al., Hum Genet. 2012 January; 131(1):87-97). Fcgr1 is a mouse ortholog of human FCGR1α (encoding the high affinity IgG Fc receptor), which was upregulated in chronic CNS lesions in MS patients, and targeting the Fc gamma receptor pathway ameliorated EAE in mice (Lock C, et al., Nat Med. 2002 May; 8(5):500-8). Lastly, Ccr1 (chemokine (C-C motif) receptor 1) has been shown to be critical for EAE pathogenesis (Rottman J B, et al., Eur J Immunol. 2000 August; 30(8):2372-7). In contrast, in males, deletion of p38α resulted in downregulation of Il10, an immunosuppressive cytokine well-known to inhibit EAE (Rott O, et al., Eur J Immunol. 1994 June; 24(6):1434-40)). Meanwhile, Il12b, the p40 subunit of IL-12 and IL-23, and known to be required for EAE (Cua D J, et al., Nature. 2003 Feb. 13; 421(6924):744-8), was upregulated.

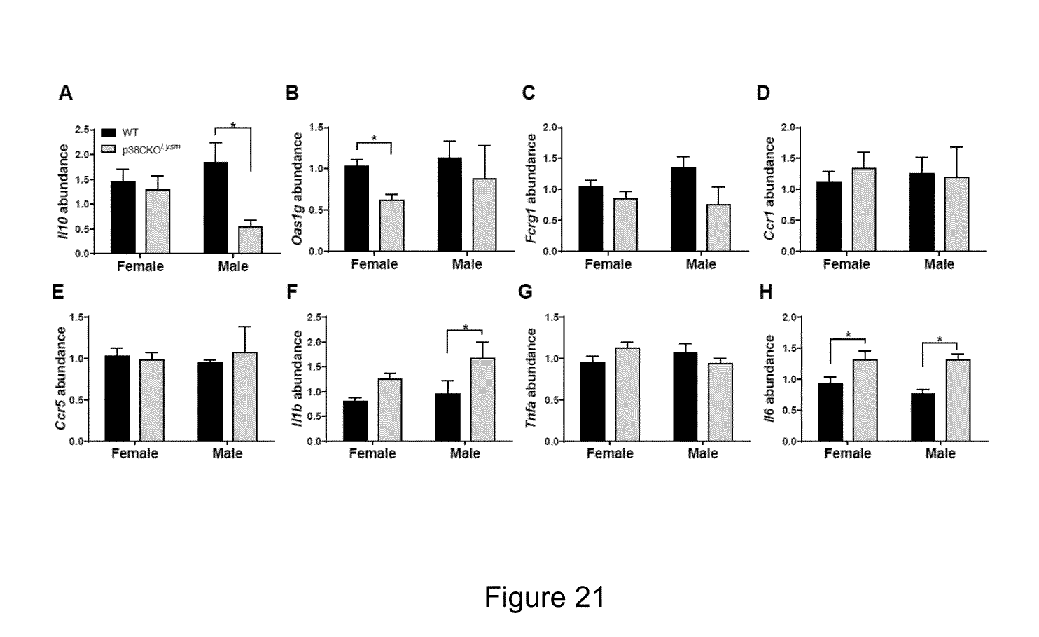

As an additional filter to determine the in vivo relevance of genes differentially regulated by p38α in macrophages stimulated in vitro, the expression of multiple transcripts was analyzed in mononuclear cells isolated from the inflamed CNS at peak EAE (day 21). Consistent with microarray results, it was found that 1110 mRNA was downregulated in cells from male but not female p38CKO^{Lysm} mice (FIG. 21A), while Oas1g mRNA was downregulated specifically in females in the absence of p38α (FIG. 21B). In contrast to the microarray results, Fcgr1, Ccr1 and Ccr5 mRNAs were unchanged in either sex (FIG. 21C-FIG. 21E), suggesting differential regulation in vivo. Several other transcripts of interest were either undetectable, or their expression was highly variable, owing likely to the heterogeneity in CNS-infiltrating cells and/or EAE timing/onset, thus precluding their analysis. The expression of several pro-inflammatory cytokines thought to be controlled by p38 MAPK was also examined. Mb, was upregulated in males in an inverse relationship with Il10 (FIG. 21F). Tnfa expression was not affected by p38α deletion, while 116 was upregulated in both females and males in the absence of p38α (FIG. 21G and FIG. 21H), similar to the results observed by ELISA in vitro (FIG. 19). Taken together, these results demonstrate that differential sex-specific regulation of pro- and anti-inflammatory gene expression by p38α underlies the opposing protective and pathogenic effects of p38 MAPK inhibition in EAE in females and males, respectively, and identify Il10 and Oas1g as key p38α-controlled sex-specific genes in the CNS during peak neuroinflammation.

TABLE 1
Non-sex-specific p38α-dependent transcripts in macrophages.

| | |
|---|---|
| female | male |

| gene | p val | FC | p val | FC |
|---|---|---|---|---|
| U90926 | 0.00 | −2.51 | 0.02 | −1.69 |
| Mmp13 | 0.00 | −2.18 | 0.00 | −1.78 |
| Il1f9 | 0.01 | −2.02 | 0.01 | −1.91 |
| Serpinb2 | 0.00 | −1.88 | 0.00 | −2.30 |
| Ccr5 | 0.01 | −1.80 | 0.00 | −1.87 |
| Lox | 0.01 | −1.57 | 0.02 | −1.51 |
| Mirlet7e | 0.04 | −1.57 | 0.05 | −1.54 |
| Ch25h | 0.00 | 1.52 | 0.00 | 1.51 |

Thioglycolate-elicited macrophages were isolated from female and male p38CKO^{Lysm} and WT mice, and stimulated with 50 μg/ml heat-killed MTB in vitro for 4 hrs. mRNA was isolated and subjected to microarray analysis to identify differentially expressed genes between p38CKO^{Lysm} and WT in both females and males. The criteria for differential expression was set at p<0.05 and signed fold change (|FC|)>1.5. Fold change indicates the change in expression in female p38CKO^{Lysm} relative to WT. Non-annotated genes are not shown.

TABLE 2
Female-specific p38α-dependent transcripts in macrophages.

| gene | p val | FC | gene | p val | FC |
|---|---|---|---|---|---|
| Retnlg | 0.021 | −2.17 | Mir187 | 0.040 | −1.58 |
| Snord82 | 0.050 | −2.12 | Tpsb2 | 0.013 | −1.58 |
| Mir20a | 0.005 | −1.93 | Ctla2a | 0.005 | −1.57 |
| Scd1 | 0.048 | −1.91 | Snord118 | 0.021 | −1.57 |
| Mir1b | 0.035 | −1.90 | Rn5s20 | 0.049 | −1.55 |
| Gpr141 | 0.006 | −1.86 | [symbol] | 0.009 | −1.54 |
| Isg15 | 0.028 | −1.80 | Scg2 | 0.015 | −1.53 |
| Trim30c | 0.002 | −1.79 | [symbol] | 0.003 | −1.52 |
| Snord15a | 0.003 | −1.77 | Dio2 | 0.021 | −1.52 |
| Cox7b | 0.038 | −1.76 | Gorab | 0.005 | −1.52 |
| Trim30d | 0.006 | −1.74 | Zfp68 | 0.008 | −1.51 |
| Syne1 | 0.011 | −1.71 | Mreg | 0.014 | −1.51 |
| Mpzl3 | 0.027 | −1.69 | Pyhin1 | 0.007 | −1.51 |
| Hist1h2bn | 0.000 | −1.65 | Tut1 | 0.021 | −1.51 |
| Hist2h3c2 | 0.004 | −1.64 | Syne1 | 0.040 | −1.50 |
| Vsig4 | 0.047 | −1.64 | Hdhd3 | 0.015 | 1.52 |
| Car2 | 0.038 | −1.64 | Zfp862 | 0.006 | 1.53 |
| Mid1 | 0.036 | −1.63 | Snora30 | 0.024 | 1.56 |
| [symbol] | 0.004 | −1.62 | Mir23a | 0.034 | 1.57 |
| Adm | 0.016 | −1.61 | Kcnj13 | 0.032 | 1.58 |
| Snord43 | 0.049 | −1.61 | Ptges31 | 0.000 | 1.61 |
| Tfrc | 0.008 | −1.61 | Pdgfb | 0.005 | 1.62 |
| Flrt3 | 0.001 | −1.61 | Kprp | 0.002 | 1.71 |
| Scarna17 | 0.011 | −1.59 | Clca2 | 0.044 | 1.78 |
| [symbol] | 0.017 | −1.59 | Mir5123 | 0.020 | 2.32 |

Microarray analysis was performed on samples collected as described for Table 1 to identify differentially expressed genes between p38CKO^{Lysm} and WT that were unique to females. The criteria for differential expression was set at p<0.05 and |FC|>1.5. Fold change indicates the change in expression in female p38CKO^{Lysm} relative to female WT. Non-annotated genes are not shown. Genes known to play a role in EAE/MS pathogenesis are italicized and bolded.

TABLE 3
Male-specific p38α-dependent transcripts in macrophages.

| gene | p val | FC |
|---|---|---|
| Mir669a-3 | 0.05 | −1.73 |
| Mup12 | 0.02 | −1.71 |
| Fpr1 | 0.03 | −1.69 |
| Fabp7 | 0.00 | −1.69 |
| Hp | 0.00 | −1.64 |
| Cd5l | 0.04 | −1.63 |
| Rrs1 | 0.02 | −1.61 |
| [symbol] | 0.04 | −1.56 |
| Mir7-2 | 0.01 | −1.53 |
| Cav1 | 0.00 | −1.52 |
| Vmn2r26 | 0.02 | −1.52 |
| Olfr1269 | 0.02 | −1.51 |
| Acpp | 0.01 | −1.51 |
| Vmn2r113 | 0.03 | −1.51 |
| Olfr566 | 0.05 | −1.51 |
| Cdc14a | 0.00 | 1.52 |
| Mir505 | 0.04 | 1.53 |
| Atp5s | 0.03 | 1.53 |
| Kansl2 | 0.01 | 1.54 |
| Pgcp | 0.01 | 1.54 |
| Calca | 0.01 | 1.55 |
| Zfp945 | 0.02 | 1.56 |
| [symbol] | 0.00 | 1.57 |
| Hgf | 0.00 | 1.60 |
| Mir215 | 0.05 | 1.73 |
| Cdr1 | 0.02 | 2.45 |

Microarray analysis was performed on samples collected as described for Table 1 to identify differentially expressed genes between p38CKO^{Lysm} and WT that were unique to males. The criteria for differential expression was set at p<0.05 and |FC|>1.5. Fold change indicates the change in expression in male p38CKO^{Lysm} relative to male WT. Non-annotated genes are not shown. Genes known to play a role in EAE/MS pathogenesis are italicized and bolded.

Sex Hormones Contribute to the Sexual Dimorphism in EAE in p38CKO^{Lysm} Mice

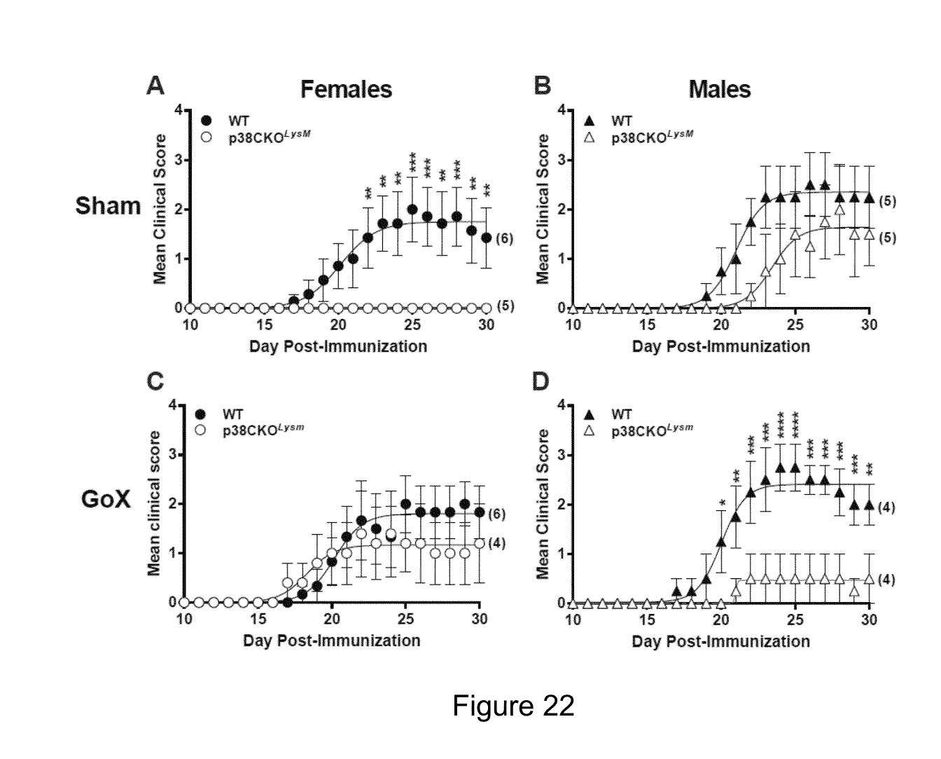

Bioinformatic analysis of differentially expressed transcript modules in macrophages from female and male p38CKO^{Lysm} mice identified estrogen as a highly significant positive upstream regulator of genes within the female-specific module (activation Z score=−2.21; p value of overlap=2.29E-04), while testosterone was a positive regulator of genes within the male-specific module (activation Z score=−1.47; p value of overlap=1.32E-06). This suggested that sex hormones may be responsible for differential EAE outcomes in female and male p38CKO^{Lysm} mice. To test this hypothesis, gonadectomies (or sham control surgeries) were performed on adult WT and p38CKO^{Lysm} mice, followed by EAE induction. Sham surgeries did not affect the sexual dimorphism, as female p38CKO^{Lysm} mice were highly resistant to EAE induction compared to WT (FIG. 22A), while EAE in p38CKO^{Lysm} males was not significantly different from WT males (FIG. 22B). However, removal of adult sex hormones by gonadectomy completely reversed the sexual dimorphism, as ovariectomized p38CKO^{Lysm} females lost EAE resistance (FIG. 22C), and orchiectomized p38CKO^{Lysm} males gained it (FIG. 22D). These results demonstrate the role of adult sex hormones in determining the sexual dimorphism in EAE pathogenesis mediated by p38α signaling in myeloid cells.

### p38 Inhibition as a Disease Modifying Therapy

In this study, it was demonstrated that deletion of p38α in T cells did not affect EAE in B6 mice (FIG. 16A and FIG. 16B). This is in contrast to previous results showing that augmentation of p38 MAPK activity by a constitutively active MKK6 transgene, or its inhibition by a dominant negative p38 transgene, expressed specifically in T cells in B10.BR mice, enhanced or diminished EAE severity, respectively (Noubade R, et al., Blood. 2011 Sep. 22; 118(12):3290-300). This discrepancy may be due to genetic differences between B6 and B10.BR mice, or the fact the transgenic approaches affect all four p38 MAPK isoforms.

The finding that SB203580 treatment did not reduce EAE in males (FIG. 15B) is somewhat counter-intuitive given reduced EAE in p38CKO^{Cd11c} males (FIG. 16D). However, based on the findings that p38CKO^{Lysm} males exhibited augmented EAE (FIG. 16F), it is predicted that pharmacological inhibition of p38 concurrently in myeloid cells and DCs has opposing effects on EAE in males which effectively cancel each other out. An alternative explanation is the involvement of other cell types targeted by SB203580 besides myeloid cells and DCs. Lastly, pharmacological experiments are difficult to compare to p38α genetic deletion studies due to effects of SB203580 on p38β or potential off-target effects (Godl K, et al., Proc Natl Acad Sci USA. 2003 Dec. 23; 100(26):15434-9).

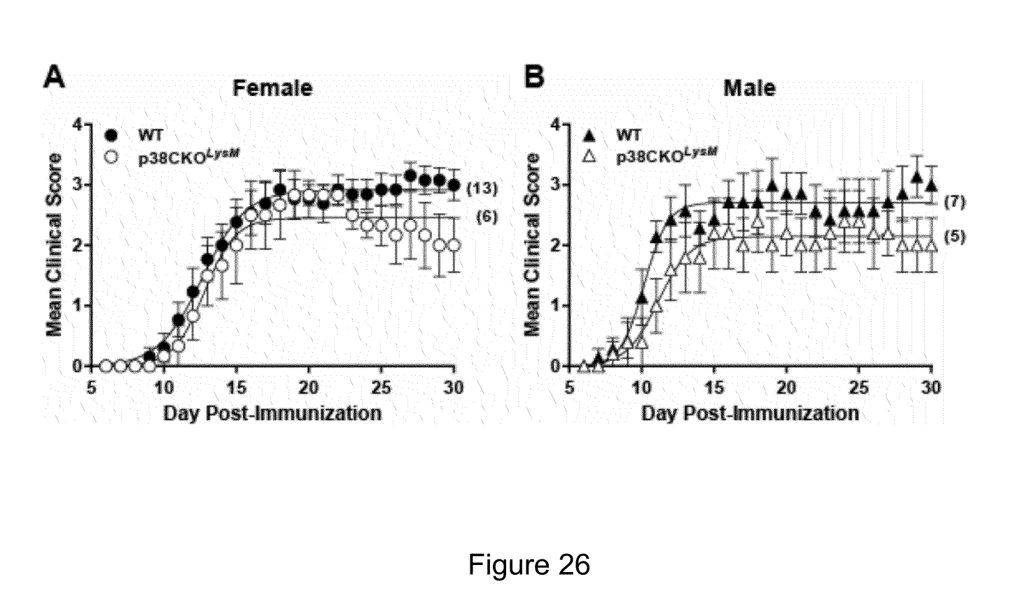

Chi and colleagues recently demonstrated that deletion of p38α in myeloid cells did not affect EAE, although the sex of the animals was not reported (Huang G, et al., Nat Immunol. 2012 February; 13(2):152-61). Another important difference is that Chi and colleagues used pertussis toxin (PTX) as an ancillary adjuvant in the EAE induction protocol, which is absent in the 2×MOG₃₅₋₅₅/CFA protocol used in the present study (FIG. 16). Because PTX overrides many genetic checkpoints (e.g., see (Spach K M, et al., J Immunol. 2009 Jun. 15; 182(12):7776-83; Blankenhorn E P, et al., J Immunol. 2000 Mar. 15; 164(6):3420-5; and Matsuki T, et al., Int Immunol. 2006 February; 18(2):399-407)), it is hypothesized that it can override disease protection provided by deletion of p38α in myeloid cells. In agreement with this, it was found that WT and p38CKO^{Lysm} female and male mice exhibited no difference in EAE disease course when the 1×MOG₃₅₋₅₅/CFA/PTX protocol was used (FIG. 26). MS exhibits remarkable heterogeneity in disease course and severity, similar to what is seen across in different EAE models elicited with or without PTX. (Blankenhorn E P, et al., J Immunol. 2000 Mar. 15; 164(6):3420-5) It is likely that different adjuvants used in EAE model different infectious/environmental risk factors in MS. Consequently, inhibitors of p38 MAPK-related pathways may have differential therapeutic efficacy depending on disease etiopathogenesis.

The present findings that deletion of p38α in macrophages altered a relatively small subset of transcripts are somewhat surprising, since p38 inhibitors were reported to inhibit wide range of proinflammatory cytokines and mediators. However, the findings are in agreement with previous reports using conditional deletion of p38α, which demonstrated that this kinase controls a limited spectrum of pro-inflammatory genes (Kim C, et al., Nat Immunol. 2008 September; 9(9):1019-27; and Guma M, et al., Arthritis Rheum. 2012 September; 64(9):2887-95). Interestingly, IL-10 production was shown to be dependent on p38α in these reports and in the present study. The differences in the results observed between pharmacologic and genetic studies may be due to the poor specificity of inhibitors (Godl K, et al., Proc Natl Acad Sci USA. 2003 Dec. 23; 100(26):15434-9). Furthermore, p38 MAPK controls many of its targets, at the post-translational level (Clark A R, et al., FEBS Lett. 2003 Jul. 3; 546(1):37-44; and Schindler J F, et al., J Dent Res. 2007 September; 86(9):800-11), including IL-17 in T cells (Noubade R, et al., Blood. 2011 Sep. 22; 118(12):3290-300), while the current analysis focused mainly on transcriptional control. Nonetheless, while it appears that effects of p38α inhibition in macrophages may be far more limited than previously suggested, it is clear that these subtle effects are sufficient to modulate EAE severity. As such, more specific and/or cell type-targeted inhibitors of p38α could represent an attractive therapeutic approach in MS.

In humans, gender influences immunity, although controversial results regarding gender and/or sex hormone treatment in ex vivo studies are frequently reported (Oertelt-Prigione S., Autoimmun Rev. 2012 May; 11(6-7):A479-85). While p38 MAPK is well-known to control pro-inflammatory functions in human monocytes/macrophages (Lee J C, et al., Nature. 1994 Dec. 22-29; 372(6508):739-46), little has been reported on gender differences in this regard. One study examined LPS-induced phosphorylation of p38 in PBMCs from males and females, and found that it was somewhat lower in females, correlating with lower proinflammatory cytokine production (Imahara S D, et al., Surgery. 2005 August; 138(2):275-82).

The incidence of MS has approximately tripled in the last 50 years, driven by an increase in relapsing-remitting disease in women (Ebers G C, Lancet Neurol. 2008 March; 7(3):268-77). This rate of change strongly suggests the existence of environmental risk factors acting at the population level in females. p38 MAPK is a well-known evolutionarily conserved component of the response to environmental stress stimuli, such as hypertonicity and UV radiation (Sheikh-Hamad D, et al., Am J Physiol Renal Physiol. 2004 December; 287(6):F1102-10; Cowan K J, et al., J Exp Biol. 2003 April; 206(Pt 7):1107-15; and Muthusamy V, et al., Arch Dermatol Res. 2010 January; 302(1):5-17). Recent epidemiological data indicate that the prevalence of MS correlates with decreased UV-radiation exposure more highly in females than it does in males (Orton S M, et al., Neurology. 2011 Feb. 1; 76(5):425-31). The immunomodulatory effects of UV-radiation (Norval M, et al., Photochem Photobiol. 2008 January-February; 84(1):19-28) are in part mediated by p38 MAPK signaling (Muthusamy V, et al., Arch Dermatol Res. 2010 January; 302(1):5-17) and UV-irradiation has been shown to influence EAE susceptibility (Hauser S L, et al., J Immunol. 1984 March; 132(3):1276-81) independent of vitamin D (Becklund B R, et al., Proc Natl Acad Sci USA. 2010 Apr. 6; 107(14):6418-23). With respect to hypertonicity, two recent reports suggested that dietary sodium may also represent an environmental risk factor for MS, as increased dietary sodium exacerbated EAE, in association with enhanced generation of pathogenic Th17 cells (Kleinewietfeld M, et al., Nature. 2013 Mar. 6; and Wu C, et al., Nature. 2013 Mar. 6). p38 MAPK appears to transduce this sodium stress signal, as inhibition of p38 MAPK abrogated sodium-induced upregulation of IL-17 production (Kleinewietfeld M, et al., Nature. 2013 Mar. 6). However, the effect of p38 MAPK inhibition on sodium-exacerbated EAE was not reported (Kleinewietfeld M, et al., Nature. 2013 Mar. 6). Since the present data show that the p38 MAPK signaling pathway is central to EAE pathogenesis in females, it is tempting to speculate that environmental stress signals acting through the p38 MAPK pathway may contribute to the increasing MS risk in females. Consequently, understanding this pathway may reveal mechanistic insight into gene-by-environment interactions in MS etiopathogenesis. Moreover, the results presented herein demonstrate that targeting the p38 MAPK signaling pathway in MS could represent a novel and much needed female-specific DMT.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of providing a gender-specific treatment of a disorder in a female subject afflicted with the disorder, said method comprising the step of administering a pharmaceutical composition comprising an effective amount of a p38 mitogen-activated protein kinase (MAPK) inhibitor to the female subject.

2. The method of claim 1, wherein the subject is a human.

3. The method of claim 1, wherein the p38 MAPK inhibitor comprises an inhibitor selected from the group consisting of antibody, intrabody, siRNA, ribozyme, antisense, aptamer, peptidomimetic, small molecule, and any combination thereof.

4. The method of claim 1, wherein the p38 MAPK inhibitor is administered to a specific cell population in the subject.

5. The method of claim 1, wherein the p38 MAPK inhibitor is administered to a myeloid cell

6. The method of claim 5, wherein the myeloid cell is selected from the group consisting of a macrophage, a microglia, a dendritic cell, and a neutrophil.

7. The method of claim 1, wherein the p38 MAPK inhibitor reduces at least one selected from the group consisting of the expression of p38 MAPK, the activation of p38 MAPK, and the activity of p38 MAPK on its effector proteins.

8. The method of claim 1, wherein the p38 MAPK inhibitor inhibits at least one isoform selected from the group consisting of p38α, p38β, p38γ, and p38δ.

9. The method of claim 1, wherein the disorder is selected from the group consisting of an autoimmune disorder, neuroinflammation, a neurodegenerative disorder, and a behavioral disorder.

10. The method of claim 9, wherein the autoimmune disorder is multiple sclerosis (MS).

11. The method of claim 1, wherein the p38 MAPK inhibitor results in decreased cytokine production.

12. The method of claim 11, wherein the decreased cytokine production is regulated on a post-transcriptional level.

13. A method of providing gender-specific prevention of a disorder in a female subject at risk for developing a disorder, said method comprising the step of administering a pharmaceutical composition comprising an effective amount of a p38 mitogen-activated protein kinase (MAPK) inhibitor to the female subject.

14. The method of claim 13, wherein the subject is a human.

15. The method of claim 13, wherein the p38 MAPK inhibitor comprises an inhibitor selected from the group consisting of antibody, intrabody, siRNA, ribozyme, antisense, aptamer, peptidomimetic, small molecule, and any combination thereof.

16. The method of claim 13, wherein the p38 MAPK inhibitor is administered to a specific cell population in the female subject.

17. The method of claim 13, wherein the p38 MAPK inhibitor is administered to a myeloid cell

18. The method of claim 17, wherein the myeloid cell is selected from the group consisting of a macrophage, a microglia, a dendritic cell, and a neutrophil.

19. The method of claim 13, wherein the p38 MAPK inhibitor reduces at least one selected from the group consisting of the expression of p38 MAPK, the activation of p38 MAPK, and the activity of p38 MAPK on its effector proteins.

20. The method of claim 13, wherein the p38 MAPK inhibitor inhibits at least one isoform selected from the group consisting of p38α, p38β, p38γ, and p38δ.

21. The method of claim 13, wherein the disorder is selected from the group consisting of an autoimmune disorder, neuroinflammation, a neurodegenerative disorder, and a behavioral disorder.

22. The method of claim 21, wherein the autoimmune disorder is multiple sclerosis (MS).

23. The method of claim 13, wherein the p38 MAPK inhibitor results in decreased cytokine production.

24. The method of claim 23, wherein the decreased cytokine production is regulated on a post-transcriptional level.
